(19) United States
(12) Patent Application Publication
(10) Pub. No.: US 2004/0236514 A1
(43) Pub. Date: Nov. 25, 2004

(54) Controlling distribution of epitopes in polypeptide sequences

(76) Inventors: Stephen C. Lee, Dublin, OH (US); Neena L. Summers, St. Charles, MO (US)

Correspondence Address: PHARMACIA CORPORATION, GLOBAL PATENT DEPARTMENT, POST OFFICE BOX 1027, ST. LOUIS, MO, 63006, US

(21) Appl. No.: 10/318,886

(22) Filed: Dec. 13, 2002

Related U.S. Application Data
(60) Provisional application No. 60/340,966, filed on Dec. 13, 2001.

(51) Int. Cl. G01N 33/50

(52) U.S. Cl. 702/19

(57) ABSTRACT

The present invention describes a novel method for the design of polypeptides with controlled distribution of immunogentic epitopes. The method allows for the controlled elimination or introductionof epitopes in consideration of major histocompatibility complex (MHC) molecule binding motifs.

### FILED OF THE INVENTION

The present invention describes a novel method for the design of polypeptides with controlled distribution of immunogenic epitopes. The method allows for the controlled elimination or introduction of epitopes in consideration of major histocompatibility complex (MHC) molecule binding motifs.

### BACKGROUND OF INVENTION

Epitopes are key determinants of immunogenicity and their presence or absence is a critical (but not sole) determinant of whether a given protein will engender an immune response. One class of epitopes are peptides possessed of specific amino acid sequence features (discussed below) that allow them to be recognized by binding proteins encoded by the major histocompatibility complex (MHC). Epitopes bound to MHC-encoded binding proteins can trigger immune responses. MHC-encoded binding proteins participate in an early step of immune recognition by binding proteins or small protein fragments (peptide epitopes) derived from pathogens or other host or non-host sources, and presenting these peptides to the cells of the immune system.

MHC molecules are classified as either Class I or Class II molecules. Class II MHC molecules are expressed primarily on cells involved in initiating and sustaining immune responses (e.g., T lymphocytes, B lymphocytes, macrophages). Class II MHC molecules are recognized by helper T lymphocytes and induce proliferation of helper T lymphocytes, which in turn mediate the amplification of immune responses to an antigen or immunogen. On the other hand, Class I MHC molecules are expressed on all nucleated cells and are recognized by cytotoxic T lymphocytes (CTLs). Among other activities, CTLs kill cells presenting specific epitopes in the context of the Class I MHC-encoded binding protein. CTLs are particularly important in tumor rejection and in fighting parasitic and viral infections. CTLs recognize their cognate antigens only in the form of peptide fragments bound to the MHC Class I molecules and not in the form of the intact, native antigen itself. Typically, the antigen is endogenously synthesized by the cell, and a portion of the protein antigen is degraded into small peptide fragments in the cytoplasm. Some of these small peptides translocate into a pre-Golgi compartment and interact with class I heavy chains to facilitate proper folding and association with the subunit beta-2 microglobulin. The peptide-MHC class I complex is then routed to the cell surface for expression and potential recognition by specific CTLs.

MHC Class I presentation of peptidic epitopes is necessary both to initiate and maintain epitope-specific cellular immune responses. Nucleated cells presenting epitopes in the context of Class I MHC complexes can be killed by cognate CTLs. Normally, suceptability of cells to specific CTL killing reflects MHC Class I presentation of an epitope associated with a pathological condition (viral or parasite infection, neoplastic growth, etc.). However, most cells presenting immunogenic epitopes on Class I lack co-stimulatory molecules required to initiate antigen-specific CTL responses. That task falls to one of several classes of cells (e.g., dendritic cells, macrophages) whose primary roles include uptake, processing and presentation of immunogenic materials to regulatory and effector cells of the immune system. These specialized cells are referred to as professional antigen presenting cells or professional APCs. All APCs presenting Class I epitopes have Class I MHC molecules on their surfaces, as well as regulatory proteins that, when an epitope is bound to the Class I molecule, activate and regulate the killing activity of cytotoxic T-cell clones that specifically recognize the epitope in its presented context.

An understanding of Class I epitope presentation is desirable for controlled, therapeutic modulation of immune responses (as by vaccines, for example), and there is considerable information available regarding peptide epitope-Class I MHC interaction. Investigations of the crystal structure of the human MHC class I molecule, HLA-A2.1 (later renamed A*020101), indicate that a peptide binding groove is created by the folding of the alpha-1 and alpha-2 domains of the class I heavy chain (Bjorkman et al., Nature 329:506 (1987). In these investigations, however, the identity of peptides bound to the groove was not determined.

The identity of peptides bound to Class I MHC can be determined by characterization of peptides eluted from the Class I MHC molecules on cell surfaces. Buus et al., Science 242:1065 (1988) first described a method for acid elution of bound peptides from MHC. Subsequently, Rammensee and his coworkers (Falk et al., Nature 351:290 (1991) have developed an approach to characterize naturally processed peptides bound to Class I molecules. Briefly, the methods involve large-scale isolation of MHC Class I molecules, typically by immunoprecipitation or affinity chromatography, from the appropriate cell or cell line (see Rotzschke and Falk, Immunol. Today 12:447 (1991).

Peptides eluted from a binding protein encoded by any given MHC Class I allele exhibit common features in their primary amino acid sequences, such as epitope length range, specific sets of amino acids occurring at specific positions in the peptide and conservation of the distances between those specific positions. Summation of these common features of the eluted peptides identifies the binding motif for the binding protein specified by the MHC Class I allele in question. Immunogenic epitopes conform to an MHC binding motif, but since different MHC-encoded binding protein allelic variants recognize different motifs, epitopes presented by one MHC-encoded binding protein may not be epitopes for others. Thus, epitopes are sometimes referred to as being epitopes of a specific MHC alelle (as being an “A₂ epitope” or a “B₇ epitope”, for instance). Definition of motifs specific for different MHC Class I alleles allows the identification of potential peptide epitopes from an antigenic protein whose amino acid sequence is known. Typically, identification of potential peptide epitopes is initially carried out by inspection of the amino acid sequence of a polypeptide of interest for the presence of motifs.

Sette et al., Proc. Natl. Acad. Sci. USA 86:3296 (1989) showed that MHC allele specific motifs in fact did predict MHC binding capacity. Schaeffer et al., Proc. Natl. Acad. Sci. USA 86:4649 (1989) showed that MHC binding was related to immunogenicity. Several authors (De Bruijn et al., Eur. J. Immunol., 21:2963-2970 (1991); Pamer et al., 991 Nature 353:852-955 (1991)) have provided evidence that class I binding motifs can be applied to the identification of potential immunogenic peptides in animal models and in humans.

In many settings (viral or other parasitic infection, cancer, etc.), specific CTL responses can be advantageous, and some vaccines are intended to induce such protective, curative or disease mitigating immune responses by stimulating antibody-mediated and/or T-cell-mediated immune responses. Prior to the last few years, immunological dogma held that peptides presented on Class I MHC are nearly exclusively derived from proteins expressed in the cells presenting the antigen, and that exogenously administered proteins were not taken up, processed and presented on Class I MHC. This dogma profoundly influenced vaccination strategies. Since it was held that exogenous proteins would not be taken up, peptide epitopes themselves were sometimes administered directly to isolated APCs (or in vivo, to patients) as vaccine entities. Exogenously administered peptides bound MHC molecules that were already on APC surfaces, and from which peptide epitopes had already dissociated. Alternatively, nucleic acid sequences encoding peptide epitopes were introduced into APCs, or other cells and immunogenic materials were expressed intracellularly, processed and presented. This could be done either by in vivo administration of epitope-encoding nucleic acids, or by introducing immunogen-encoding nucleic acids to cells in vitro, followed by reintroduction of the cells expressing the immunogen to the patient or host. All of these approaches have significant limitations, mostly relating to the low efficiency with which these methods mediated presentation of immunogenic epitopes, the need for extensive ex vivo manipulation of patient cells, and/or unfavorable pharmacokinetic and pharmacodynamic properties associated with these classes of vaccine entities.

The ground-breaking work of Kenneth Rock and colleagues (reviewed in S. Raychaudhuri and K. L. Rock, 1998, Nature Biotechnology 16:1025-1031) and other investigators demonstrated the fallacy of the dogma that intracellular expression of antigens was absolutely required for processing of antigens to generate MHC Class I-presented immunogenic epitopes. They showed that exogenously administered antigenic polypeptides could indeed be taken up by APCs, and that these antigens when processed to immunogenic epitopes, were presented on Class I MHC complexes and triggered specific CTL responses. These critical observations facilitated polypeptide and protein vaccines to engender CTL responses, and removed the absolute requirement for problematic peptide vaccine and DNA vaccine approaches.

Native protein antigens themselves can be used in vaccines, but knowledge of MHC Class I binding motifs allows multiple epitopes, perhaps derived from multiple antigenic proteins, to be identified and incorporated into a single designed polypeptide sequence. These so-called polyepitopes offered the superior pharmacokinetic/pharmacodynamic properties of proteins (as opposed to those of peptides or nucleic acids) as well as the vaccine formulation convenience inherent in the incorporation of multiple epitopes in a single molecular entity by linking them together in a single polypeptide chain. However, given the nature of MHC Class I binding motifs, each epitope-epitope juncture can potentially incorporate multiple new epitopes (so-called junctional epitopes) which begin in an upstream epitope and end in a downstream epitope. Such junctional epitopes are artifacts of linking multiple epitopes together, and do not contribute to a therapeutic immune response, since they are present in the polyepitope, but not in the native antigens from which the epitopes of the polyepitope were taken. Since multiple binding motifs might span a single epitope-epitope juncture in a polyepitope, junctional epitopes can easily outnumber the intended vaccine epitopes of the polyepitope and potentially diminish the immune response to the intended epitopes by competing with the vaccine epitopes for processing or presentation of molecules and structures (Perkins et al., 1991 J. Immunol. 146: 2137-2144). The ability of junctional epitopes to compete with desired epitopes in generation of immune responses has been documented for both MHC Class I restricted responses(CTLs, e.g., Tussey et al. 1995. Immunity 3: 65-77) and MHC Class II-restricted responses (antibodies, Wang, Y., et al. 1992 Cell Immunol 143: 284-297). Nucleic acid vaccines can encode polyepitopes instead of native protein antigens, and the same considerations around junctional epitopes that apply to exogenously produced polypeptides apply as well to polyepitopes expressed intracellularly from vaccinating nucleic acid segments. Minimizing junctional epitopes within synthetically or biologically produced polyepitopes, or within polyepitopes encoded by nucleic acid segments, has the potential benefit of reducing competing, non-therapeutic immune responses and thereby augmenting desired immune responses.

To date, knowledge of MHC Class I binding motifs has not been systematically applied to design vaccines containing only epitopes relevant to the particular disease state the vaccine is directed to. In fact, many vaccination strategies use remarkably crude biological preparations, such as intact virus particles, cells, cellular extracts, etc., that are often not fully defined. These vaccines often generate both antibody- and cell-mediated immunity, and do not allow one to modulate the qualities of the immune response generated. The crudity of many current vaccines can lead to ineffective or inappropriate immune responses that in some settings might be therapeutically deleterious.

The discussion to this point has focused on therapeutic interventions intended to produce beneficial immune responses. However, immune responses elicited by some antigens can be pathological, and a number of autoimmune disease states (disease states that involve immune responses directed to self-antigens) are known. Unfortunately, the etiology of many autoimmune responses have not been fully elucidated, though at least some such responses are thought to arise as the result of specific immune responses to immunogenic epitopes in exogenous antigens (e.g., pathogens, foods, environmental allergens, therapeutics, vaccines) which are cross-reactive (i.e., recognized by the same immune effectors) as are epitopes of self-antigens.

The clinical consequences of autoimmune disorders can be devastating. Autoimmune-associated disorders include, for example, multiple sclerosis (MS), rheumatoid arthritis (RA), Sjogren syndrome, scleroderma, polymyositis, dermatomyositis, systemic lupus erythematosus, juvenile rheumatoid arthritis, ankylosing spondylitis, myasthenia gravis (MG), bullous pemphigoid (antibodies to basement membrane at the dermal-epidermal junction), pemphigus (antibodies to mucopolysaccharide protein complex or intracellular cement substance), glomerulonephritis (antibodies to glomerular basement membrane), Goodpasture's syndrome, autoimmune hemolytic anemia (antibodies to erythrocytes), Hashimoto's disease (antibodies to thyroid), pernicious anemia (antibodies to intrinsic factor), idiopathic thrombocytopenic purpura (antibodies to platelets), Grave's disease, and Addison's disease (antibodies to thyroglobulin), and the like. In some cases the specific self-antigens, and in some cases the specific peptide antigens, to which pathological autoimmune responses are directed is known, but more often the self antigen(s), and, perhaps more importantly from a therapeutic and prevention standpoint, nonself-antigen(s) that may trigger autoimmune responses are not fully identified and defined.

One cannot rule out the possibility that responses to junctional epitopes of polyepitope vaccines might be cross-reactive to self-antigens and induce autoimmune responses. This consideration makes the ability to design proteins such that their immunogenic epitope distribution is controlled all the more important. Furthermore, since knowledge of all human self-antigens is incomplete, any immunogenic epitope of a heterologous protein cannot be excluded as being potentially cross-reactive with respect to a self-antigen. Also, there has been much public concern over the possibility of introducing antigenic proteins into the food supply in the genetic modification of plants and animals (GMO foods). Unfortunately, while the composition of some environmental antigens are under human control (i.e., those antigens that are produced using biotechnology or synthetic chemistry means), there currently exists no design and production strategy that affords control of epitope composition of potential immunogens produced using human technologies.

The converse of inadvertantly triggering immune responses by therapeutic administration is the administration of a therapeutic to mitigate specific immune responses. Such therapeutics are typically protein moieties containing self-antigen epitopes or their equivalents, and are intended to induce or re-induce tolerance to self- or environmental antigens, thereby silencing deliterious immune responses. However, strict control of the immunogenic epitope content of a polypeptide is currently unavailable, and the current therapeutic capacity to suppress only immune responses that are deliterious, while leaving beneficial responses unperturbed using tolerance (re)induction strategies is limited.

Thus, the requirements for peptide binding to many human Class I MHC proteins have been determined empirically, and a positive correlation between binding to Class I MHC and the capacity of an epitope to trigger specific CTL responses has been clearly demonstrated. Furthermore, epitope-specific CTL responses are beneficial in some settings (e.g., infections by viruses or other parasites, cancer) and deliterious in others (autoimmune conditions or other epitope-specific immune responses to environmental antigens). The benefits of a design method to control epitope distribution within engineered or naturally-occurring polypeptide sequences both to maximize desired immune responses, and to minimize, avoid or suppress undesired responses would seem to be large. Despite these considerations, a polypeptide design method that systematically exploits understanding of the biology of MHC Class I-restricted immune responses to control of the distribution of MHC Class I epitopes within a polypeptide sequence has not yet been described.

### SUMMARY OF THE INVENTION

In one embodiment, the present invention relates to compositions and methods for preventing, treating or diagnosing a number of pathological states.

In another embodiment, the present invention allows control of epitope number and position in an engineered polypeptide sequence through systematic consideration of MHC Class I binding motifs.

In yet another embodiment, the invention is a method that designs polypeptides in consideration of motif data. The method is directed to the systematic manipulation of Class I MHC binding motif data in polypeptide design.

In one embodiment, the instant invention applies a method to potential polypeptide sequences that systematically takes into account available MHC Class I binding motifs, identifies exhaustively all potential variant sequences of a parental polypeptide sequence that lack epitopes recognized by the binding protein encoded by any given set of MHC Class I alleles. In another embodiment, the instant invention applies a method to potential polypeptide sequences that systematically takes into account available MHC Class I binding motifs, identifies exhaustively all potential variant sequences of a parental polypeptide sequence that contain epitopes recognized by the binding protein encoded by any given set of MHC Class I alleles. The present design method achieves control of epitope distribution by manipulation of the amino acid sequences of the polypeptide under design. Within the bounds of Class I MHC binding motifs, the present invention identifies all amino acid sequences which satisfy the binding motif distribution constraints determined by the designer.

In one embodiment, epitopes are eliminated by violating the constraints of MHC binding motifs, in by substituting some or all of the N-terminal, intermediate, or C-terminal anchor residues (or combinations thereof) with amino acids other than those allowed as anchors.

In one embodiment, epitopes can also be eliminated by directed alteration of spacing between existing anchor residues. In another embodiment, epitopes can be introduced into polypeptide sequences under design by introducing amino acid segments to satisfy motif constraints between anchor residues. Epitopes can be introduced by insertion of or substitution with N-terminal, intermediate, or C-terminal anchor residues anchor residues at distances from one another dictated by an MHC Class I binding motif.

In one embodiment, epitopes can be introduced by inserting or substituting an N-terminal, intermediate or C-terminal anchor residue at a distance from an existing C-terminal, intermediate or N-terminal anchor in a polypeptide sequence at a position dictated by a MHC Class I binding motif.

In one embodiment, epitopes can be introduced by satisfying the spacing requirements dictated by a Class I MHC binding motif by amino acid insertion or deletion between N— and C-terminal anchor residues already present in the polypeptide. In another embodiment, the present method allows the design of multiple, related polypeptides exhibiting pre-determined distributions of MHC Class I binding motifs. Application of the instant invention identifies all possible variant amino acid sequences of a parent polypeptide that exhibit a specific, pre-determined distribution of immunogenic epitopes.

In one embodiment, the present invention allows design of all variants of a parental polypeptide that exhibit, in addition to its pre-determined epitope content, some other property determined by their primary amino acid sequence. These include, but are not limited to enzymatic activity, ability to be expressed, receptor agonist activity, the ease of synthesis, expression, formulation, storage, or delivery. In another embodiment, the design property is selected from charge distribution, pI, hydrophobicity, aggregation/particle size, N—,C-terminal segment codon/amino acid preferences, local residue order, bias for optimal proteosomal processing and loading, coupling sites, and post-translational modifications.

Polypeptides exhibiting the target epitope distributions can be made and screened for the desired enzymatic or other activity by any of a number of methods familiar to those skilled in protein biochemistry, in order to identify a variant with both the requisite activity and the desired epitope distribution.

In yet another embodiment, the present invention allows design of polypeptides to which immune responses would be directed towards selected areas of said polypeptides. In another embodiment, the present invention allows design of polypeptides to which immune responses would be directed away from selected areas of said polypeptides. In another embodiment, the present invention allows design of polypeptides selectively modulating the immunogenicity of protein or supramolecular protein structures for individuals or organisms having specific MHC Class I allelic complements.

In one embodiment, the present invention allows design of protein vaccines that lack undesired epitopes. In another embodiment, the present invention allows design of polyepitope vaccines that lack undesired epitopes. In yet another embodiment, the present invention allows design of polyepitope vaccines that lack undesired epitopes occurring at the junctions between the vaccine epitopes.

In one embodiment, polypeptides congruent with a particular therapeutic aim could be designed specifically for individuals in consideration of the MHC Class I allelic content of those individuals.

In another embodiment, therapeutic polypeptides will be designed for larger groups of individuals, in consideration of the distribution of Class I MHC alleles in the target population. The present invention considers the distribution of different MHC Class I alleles to design polypeptide sequences suitable for populations exhibiting particular ethnic demographics. Such populations may reflect those of particular nations or those of target market groups.

In yet another embodiment, the method could be used to design therapeutics for use within subpopulations of specific ethnic or racial groups: for example, those members of the population whose lifestyles or environments expose them to risk of development of particular disease states or encountering particular pathogens or environmental antigens. At risk subpopulations may be identified by any of a number of demographic, geographic, environmental, ecological, behavioral, ethnographical, cultural, epidemiological, toxicological, anthropological, physical, genetic, biochemical, immunological, medical, therapeutic or other criteria, and the present invention can be used to design safe and efficacious therapeutics in consideration of the distribution of MHC Class I alleles within the so-identified subpopulations.

### DETAILED DESCRIPTION OF INVENTION

The following detailed descriptions are presented for illustrative purposes only and are not intended as a restriction on the scope of the invention. Rather, they are merely some of the embodiments that one skilled in the art would understand from the entire contents of this disclosure. All parts are by weight and temperatures are in Degrees centigrade unless otherwise indicated.

The following is a list of abbreviations and the corresponding meanings as used interchangeably herein:

mg=milligram

ml or mL=milliliter

μg or ug=microgram

μl or μL or μl or uL=microliter

The following is a list of definitions of various terms used herein:

The term “addition” means to increase the number or amount.

The term “allergenicity” means the property of a substance to induce an allergic response in a sensitive individual.

The term “altered” means that expression differs from the expression response of cells or tissues not exhibiting the phenotype.

The term “amino acid” as used herein includes proteins, protein fragments, linked amino acids, e.g., residues, and individual amino acids, including any of the naturally-occurring carboxylic amino acids, D- and L-optical isomers and racemic mixtures thereof, synthetic amino acids, and derivatives of these natural and synthetic amino acids. Amino acids can be symbolically represented.

The term “anchor”, “anchor residue” or “anchor position” means the amino acid(s)of a peptide that binds the peptide-binding groove of an MHC molecule.

The term “antibody” means a protein belonging to the class of immunoglobulins which binds specifically to a particular substance.

The term “antigen” means a substance that is recognized specifically by an antibody or specific cytotoxic lymphocyte.

The term “antigen presenting cells” or “APCs” means a type of specialized cell that can process antigens and display their peptide fragments on the cell surface, together with molecules required for lymphocyte activation. APCs include dendritic cells, macrophages, B-cells, Langerhans' cells, monocytes, and follicular dendritic cells.

The term “antigenicity” means a molecule that triggers generation of either a humoral or cellular immune response.

The term “binding motif” or “MHC binding motif” or “motif” means a canonical amino acid sequence of defined length that incorporates amino acid residues at particular sites in the sequence with obligate spacing between them such that any amino acid sequence that satisfies the constraints of the binding motif can bind MHC molecules.

The term “cellular immune response” means any adaptive immune response in which antigen specific T-cells have the main role.

The term “complete complementarity” means that every nucleotide of one molecule is complementary to a nucleotide of another molecule.

The term “controlling distribution” means delimiting epitope position and frequency in a polypeptide.

The term “C-terminus” means the last C-most in relative order of a particular peptide sequence, including epitope order, anchor order, spacer order, and amino acid order.

The term “degenerate” means that two nucleic acid molecules encode for the same amino acid sequences but comprise different nucleotide sequences.

The term “epitope” means a peptide sequence capable of eliciting an immunogenic response.

The term “exogenous genetic material” means any genetic material, whether naturally occurring or otherwise, from any source that is capable of being introduced into any organism.

The term “expansion” means the differentiation and proliferation of cells.

The term “fusion protein” means a protein or fragment thereof that comprises one or more additional peptide regions not derived from that protein.

The term “haplotype” means the portion of the MHC phenotype determined by a set of genes inherited from a parent.

The term “HLA” means human lymphocyte antigens.

The term “immunity” means the ability to resist infection.

The term “immunoaffinity” means techniques by which materials are isolated by virtue of their content of immunogenic epitopes.

The term “immunogenicity” means the property of a material to elicit an immune response.

The term “immunotherapy” means a treatment modality that affects physiological changes in a target animal using the immune system.

The term “introducing” means the insertion of amino acid(s) within a pre-existing amino acid sequence.

The term “junctional epitope” means an epitope, which spans across two or more desired epitopes in a polyepitope polypeptide.

As used herein, “linker” or “spacer” refers to a molecule or group of molecules that connects two molecules. An amino acid linker means zero or more amino acids that connect two polypeptides.

The term “MHC” or “major histocompatibility complex” means a set of linked genes that encode proteins involved in antigen processing and other aspects of host defense specifically including the cell surface proteins that are involved in antigen presentation.

The term “MHC class” means a set of MHC molecules that are capable of antigen presentation to cytotoxic T-cells (class I MHC) or to helper T-cells (class II MHC).

The term “MHC class I” or “class I MHC” means the cell surface protein which binds and presents epitopes to cytotoxic T-cells.

The term “MHC class II” or “class II MHC” means the cell surface protein, which binds and presents epitopes to helper T-cells.

The term “linker” or “amino acid linker” means a linear series of amino acids inserted into a polypeptide sequence to control epitope distribution.

The term “N-terminus” means the first N-most in relative order of a particular peptide sequence, including epitope order, anchor order, spacer order, and amino acid order.

The term “pattern-matching” means a string comparison (comparing one string of characters to another string of characters). It is understood that strings do not have to be of equal length.

The term “peptide” or “peptide fragment” or “polypeptide” means a compound of two or more amino acids in which a carboxyl group of one is united with an amino group of another, forming a peptide bond with an (amino) N-terminus and a (carboxyl) C-terminus. The term “polypeptide” includes full-length proteins, and processed and folded proteins.

The term “phenotype” means any of one or more characteristics of an organism, tissue, or cell.

The term “polyepitope” means a designed polypeptide sequence containing multiple epitopes linked together.

The term “probe” means an agent that is utilized to determine an attribute or feature (e.g. presence or absence, location, correlation, etc.) of a molecule, cell, tissue, or organism.

The term “protein fragment” means a peptide or polypeptide molecule whose amino acid sequence comprises a subset of the amino acid sequence of that protein.

The term “protein molecule/peptide molecule” means any molecule that comprises seven or more amino acids.

The term “recombinant” means any agent (e.g., DNA, peptide, etc.), that is, or results from, however indirectly, human manipulation of a nucleic acid molecule.

The term “removing immunogenic epitopes” means elimination of amino acid sequences satisfying MHC Class I binding motifs in a polypeptide sequence by any of a number of means taught herein.

The term “specifically bind” means that the binding of an antibody or peptide is not competitively inhibited by the presence of non-related molecules.

The term “substitute” or “substitution” means replacement of one amino acid with another in a polypeptide sequence.

The term “synthetic peptide” or “synthetic polyepitope” means a polyepitope of peptide made by chemical synthesis.

The term “T-cell” means a subset of lymphocytes defined by their development in the thymus and which recognize specific epitopes presented in the context of binding to MHC and which will kill cells presenting those epitopes (cytotoxic T-cells) or which amplify the responses of other effector cells (helper T-cells) by recognizing presentation of specific epitopes in the context of MHC molecules and providing cells so presenting those epitopes with growth and differentiation signals.

The term “vaccine” means a substance or a cell intended to stimulate a desired immunological response or mitigate or prevent an undesired immune response.

Class I MHC Molecules and Peptide Binding Motifs

The major histocompatibility (MHC) locus encodes a variety of proteins which regulate and effectuate immune responses, including the Class I MHC antigens (also called MHC Class I molecules). Class I MHC molecules are integral membrane proteins that bind peptide fragments derived from antigens or immunogens and “present” the peptides to cytotoxic T-cells as immunogenic epitopes. Depending on the cellular context in which epitope presentation occurs, different outcomes accrue from epitope presentation on Class I molecules. Professional antigen presenting cells (APCs) provide co-stimulatory signals, in addition to an epitope presented in the context of MHC Class I molecules, that activate resting cytotoxic T-cells that specifically recognize the presented epitope, initiating a cellular immune response. In the case of many other nucleated cells, epitope presentation takes place in the absence of co-stimulatory cells, and the cells are killed by the action of activated T-cells cognate for the presented epitope.

Class I MHC molecules are thus key regulators of cellular immunity. The MHC class I antigens are polygenic: that is, they are encoded by multiple genes, specifically the HLA-A, B, and C loci. HLA-A and B antigens are expressed at the cell surface at approximately equal densities, whereas the expression of HLA-C is significantly lower (perhaps as much as 10-fold lower). The individual HLA loci are themselves also highly polymorphic, meaning that each of these loci have a number of alleles.

Peptide epitopes manipulated using the present invention preferably conform to a motif recognized by an MHC I molecular species having a wide distribution in the human population. Since the MHC alleles occur at different frequencies within different ethnic groups and races, the choice of target MHC allele may depend upon the target population. Table 1 shows the frequency of various alleles at the HLA-A locus products among different races.

TABLE 1

| A Allele/Subtype | N(69)* | A(54) | C(502) |
|---|---|---|---|
| A*010101 | 10.1(7) | 1.8(1) | 27.4(138) |
| A*020101 | 11.5(8) | 37.0(20) | 39.8(199) |
| A*0203 | 1.4(1) | 5.5(3) | 0.8(4) |
| A*0211 | — | — | — |
| A*030101 | 1.4(1) | 0 | 0.2(0) |
| A*002 | 5.7(4) | 5.5(3) | 21.5(108) |
| A*110101 | 0 | 5.5(3) | 0 |
| A*1102 | 5.7(4) | 31.4(17) | 8.7(44) |
| A*1103 | 0 | 3.7(2) | 0 |
| A*24020101 | 2.9(2) | 27.7(15) | 15.3(77) |
| A*2601 | 4.3(3) | 9.2(5) | 5.9(30) |
| A*2602 | 7.2(5) | — | 1.0(5) |
| A*2902 | 10.1(7) | 1.8(1) | 5.3(27) |
| A*3002 | 1.4(1) | — | 0.2(1) |
| A*3001 | 7.2(5) | — | 3.9(20) |

Table 1. Since Class I MHC alleles occur at different frequencies within different ethnic groups and races, the choice of target MHC allele may depend on which ethnic population is the intended recipient of the engineered polypeptide. The majority of the Caucasoid population can be covered by peptides which bind to four HLA-A allele subtypes, specifically HLA-A*020101, A*010101, and A*0302. Similarly, the majority of the Asian population is encompassed with the addition of peptides binding to a fifth allele HLA-A*1102. Table compiled from B. DuPont, Immunobiology; of HLA, Vol. 1, Histocompatibility Testing 1987, SpringerVeriag, New York 1989. N=negroid; A=Asian; C=caucasoid. Numbers in parenthesis represent the number of individuals included in the analysis.

Thus, in any given instance, the instant design method is used in the context of binding motifs of some particular set of MHC molecules encoded by a specific set of Class I MHC alleles, and the specific set of Class I MHC alleles that will be considered to design any polypeptide will be chosen by the designer. Specifically, identification of target populations, identification of the appropriate distribution of MHC Class I alleles, and therefore the MHC Class I binding motifs that the method will consider in any given instance is particular to each individual polypeptide design project. The instant method is directed to design of polypeptide sequences that exhibit the distribution of epitopes satisfying binding motif parameters of a set of MHC Class I molecules encoded by a particular set of MHC Class I alleles chosen by each designer for any reason.

While the different allelic variants of MHC Class I molecules are distinct in their capacity to recognize different peptide binding motifs, the MHC Class I molecules on the surface of APCs and other nucleated cells are similar to each other in structure and exist as part of complexes on the cell surface. Specifically, Class I MHC complexes on cell surfaces consist of two polypeptide chains, the larger of which (the alpha or heavy chain) is a membrane spanning protein of about 43,000 Da, and is encoded by the MHC. The alpha subunit contains the peptide binding cleft of the MHC Class I molecule, determines the specific binding motif recognized by the Class I MHC complex and corresponds to the subtypes shown in Table 1. The second polypeptide is beta-2 microglobulin, and is neither directly involved in peptide epitope binding nor is it encoded by the MHC.

The three-dimensional shape of the Class I MHC complex has two domains of alpha chain (called alpha-1 and alpha-2) forming a binding cleft. The binding cleft itself is bounded by anti-parallel alpha helixes (contributed by each of the two domains, alpha-1 and alpha-2) that lie over a ‘floor’ consisting of two sets of anti-parallel beta strands (one set contributed by the alpha-1 and one set contributed by the alpha-2 domain).

Peptide epitopes are bound between the alpha helices, with their N— to C-axis running roughly parallel to one of the alpha helices, and above the beta strands, in the MHC class I binding cleft formed by the alpha helices and beta strands. The structure of the cleft constrains the peptides that Class I MHC molecules can bind, in one embodiment of the invention, to sequences of 8-11 amino acids in length. Contact between the MHC Class I heavy chain and the epitopes bound to it occur between bound peptide and the alpha helices of the binding cleft. In one embodiment, contact to the MHC Class I binding cleft occur at or near the amino and carboxy ends of the bound peptide. For any given allelic variant of Class I MHC heavy chain (for instance, as for those specified in Table 1), the position of these contacts are conserved, and when the amino acids are numbered from the amino end of the bound peptide, occur, in one embodiment, at amino acid position 2 and the most C-terminal amino acid of the bound peptide. These conserved positions are referred to as ‘anchor’ positions (as in N-terminal and C-terminal anchors), and for each allelic variant of the MHC Class I heavy chain, there is a set of amino acids which are allowed at each anchor positions. Often, but not always, these are hydrophobic or basic amino acids. In any event, the specific sets of amino acids allowable at the N-terminal and C-terminal anchor positions are specific for each individual allelic variant of the MHC Class I heavy chain. Taken together, the specific constraints on the sequence of the peptides that can be bound to any given MHC Class I heavy chain constitute the binding motif for that MHC molecule.

Specific binding motifs are determined empirically, in one embodiment, by characterization of peptides eluted from MHC Class I molecules. The procedures used to identify motifs are, in one embodiment, as described in Falk et al., Nature 351:290 (1991), which is incorporated herein by reference. Briefly, the methods involve large-scale isolation of MHC class I molecules, typically by immunoprecipitation or affinity chromatography, from the appropriate cell or cell line. Examples of other methods for isolation of the desired MHC molecule equally well known to the artisan include ion exchange chromatography, lectin chromatography, size exclusion, high performance ligand chromatography, and a combination of all of the above techniques.

Epitope characterization strategies often involve use of MHC Class I molecules whose allelic identity is known. A large number of cells with defined MHC molecules, particularly MHC Class I molecules, are known and readily available. For example, human EBV-transformed B cell lines have been shown to be excellent sources for the preparative isolation of class I and class TI MHC molecules. Well-characterized cell lines are available from private and commercial sources, such as American Type Culture Collection (“Catalogue of Cell Lines and Hybridomas,” 6th edition (1988) Rockville, Md., U.S.A.); National Institute of General Medical Sciences 1990/1991 Catalog of Cell Lines (NIGMS) Human Genetic Mutant Cell Repository, Camden, N.J.; and ASHI Repository, Brigham and Women's Hospital, 75 Francis Street, Boston, Mass. 02115. Table 2 lists some B cell lines suitable for use as sources for HLA-A alleles. All of these cell lines can be grown in large batches and are therefore useful for large scale production of MHC molecules. One of skill will recognize that these are merely exemplary cell lines and that many other cell sources can be employed. Similar EBV B cell lines homozygous for HLA-B and HLA-C could serve as sources for HLA-B and HLA-C alleles, respectively.

TABLE 2
HUMAN CELL LINES (HLA-A SOURCES)

| HLA-A allele | B cell line |
|---|---|
| A1 | MAT |
| | COX (9022) |
| | STEINLIN (9087) |
| A2.1 | JY |
| A3.2 | EHM (9080) |
| | HO301 (9055) |
| | GM3107 |
| A24.1 | KT3 (9107), |
| | TISI (9042) |
| A11 | BVR (GM6828A) |
| | WT100 (GM8602) |
| | WT52 (GM8603) |

Immunoprecipitation can be used to isolate the desired allelic variant of the MHC Class I molecule. A number of protocols can be used, depending upon the specificity of the antibodies used. For example, allele-specific mAb reagents can be used for the affinity purification of the HLA-A, HLA-B, and HLA-C molecules. Several mAb reagents for the isolation of HLA-A molecules are available (Table 3). Thus, for each of the targeted HLA-A alleles, reagents are available that may be used for the direct isolation of the HLA-A molecules. Affinity columns prepared with these mAbs using standard techniques are successfully used to purify the respective HLA-A allele products.

In addition to allele-specific mAbs, broadly reactive anti-HLA-A, B, C mAbs, such as W6/32 and B9.12.1, and one anti-HLA-B, C mAb, B 1.23.2, could be used in alternative affinity purification protocols.

TABLE 3
ANTIBODY REAGENTS

| anti-HLA | Name |
|---|---|
| HLA-A1 | 12/18 |
| HLA-A3 | GAPA3 (ATCC, HB122) |
| HLA-11, 24.1 | A11.1M (ATCC, HB164) |
| HLA-A, B, C | W6/32 (ATCC, HB95) |
| monomorphic | B9.12.1 (INSERM-CNRS) |
| HLA-B, C | B.1.23.2 (INSERM-CNRS) |

Immunopercipitation does not dissociate peptides from MHC Class I molecules, and the peptides can be eluted, harvested and characterized. The peptides bound to the peptide binding groove of the isolated MHC molecules can be eluted using acid treatment. Peptides can also be dissociated from class I molecules by a variety of standard denaturing means, such as heat, pH, detergents, salts, chaotropic agents, or a combination thereof.

Peptide fractions are further separated from the MHC molecules by reversed-phase high performance liquid chromatography (HPLC) and sequenced. Peptides can be separated by a variety of other standard means well known to the artisan, including filtration, ultrafiltration, electrophoresis, size chromatography, precipitation with specific antibodies, ion exchange chromatography, isoelectric focusing, and the like.

Sequencing of the isolated peptides can be performed according to standard techniques such as Edman degradation (Hunkapiller, M. W., et al., Methods Enzymol. 91, 399 [1983]). Other methods suitable for sequencing include mass spectrometry sequencing of individual peptides as previously described (Hunt, et al., Science 225:1261 (1992), which is incorporated herein by reference). Amino acid sequencing of bulk heterogeneous peptides (e.g., pooled HPLC fractions) from different class I molecules typically reveals a characteristic sequence motif for each class I allele. Some investigators have reported successful amino acid sequencing of abundant peptide epitopes in various HPLC fractions by conventional automated sequencing of peptides eluted from Class I molecules of the B type (Jardezky et a]., Nature 353: 326 (1991)) and of the A2.1 type by mass spectroscopy (Hunt et al., Science 225: 1261 (1992).

Thus, each MHC Class I binding motif specifies sets of amino acids that are acceptable as N-terminal anchor residues, sets of amino acids that are acceptable as intermediate anchor residues, or sets of amino acids that are acceptable as C-terminal anchor residues as well as spacing between N-terminal and C-terminal anchor residues. In one embodiment, the N-terminal anchor occurs at amino acid position 2 or 3 of MHC Class I epitopes, while the C-terminal anchor occurs at the most C-terminal amino acid of MHC Class I epitopes (position 9 or 10). Whatever the parameters of a particular MHC Class I binding motif, they must be satisfied for a given 8-11 amino acid peptide to be presented on the corresponding MHC Class I binding proteins and, in that context, to trigger an epitope-specific, MHC Class I restricted CTL response.

N-terminal, C-terminal, and intermediate anchors may occur at many positions, which re statistically relevant binding patterns that can be described as a motif. Many other such patterns are known in the art. For example, Rammensee et al., Molecular Biology Intelligence Unit: MHC Ligands and Peptide Motifs, Chapman & Hall (1997) (herein encorporated by reference in its entirety), describes many MHC motifs.

Vaccine Compositions

Vaccines are therapeutic entities which are used to modulate immune responses, by either triggering desired immune responses, or mitigating or preventing undesired responses. There are many types of vaccines, including live, attenuated or killed pathogens or host cells, genetically altered pathogens or host cells, immunogenic subunits of pathogens, pathological host cells or macromolecules made in a heterologous host, dead or live host cells pre-treated with immunogenic fractions of pathogens, pathological host cells or macromolecules, peptidic epitopes themselves or engineered polypeptides whose sequences include immunogenic epitopes. In one embodiment, the immunogenic component of the vaccine contains peptide epitopes (or proteins containing peptide epitopes within their sequences) that can be presented, if appropriately processed, on Class I or Class II MHC molecules.

Immunogenic proteinacious components of vaccines can be prepared synthetically (Merrifield, Science 232:341-347 (1986), Barany and Merrifield, The Peptides, Gross and Meienhofer, eds. (New York, Academic Press), pp. 1-284 (1979); and Stewart and Young, Solid Phase Peptide Synthesis, (Rockford, Ill., Pierce), 2d Ed. (1984)), incorporated by reference herein. Vaccine polypeptides of the invention can be prepared in a wide variety of ways. They can be synthesized in solution or on a solid support in accordance with conventional techniques. Various automatic synthesizers are commercially available and can be used in accordance with known protocols. See, for example, Stewart and Young, Solid Phase Peptide Synthesis, 2d. ed., Pierce Chemical Co. (1984).

Vaccine polypeptides can also be made using recombinant DNA technology or isolated from natural sources such as whole viruses or tumors. Recombinant DNA technology may be employed wherein a nucleotide sequence which encodes an immunogenic peptide of interest is inserted into an expression vector, transformed or transfected into an appropriate host cell and cultivated under conditions suitable for expression. These procedures are generally known in the art, as described generally in Sambrook et al., Molecular Cloning, A Laboratory Manual, Cold Spring Harbor Press, Cold Spring Harbor, N.Y. (1982), which is incorporated herein by reference. Thus, fusion proteins can be used to present the appropriate T cell epitope.

As the coding sequence for peptides of the length contemplated herein can be synthesized by chemical techniques, for example, the phosphotriester method of Matteucci et al., J. Am. Chem. Soc. 103:3185 (1981), modification can be made simply by substituting the appropriate base(s) for those encoding the native peptide sequence. The coding sequence can then be provided with appropriate linkers and ligated into expression vectors commonly available in the art, and the vectors used to transform suitable hosts to produce the desired fusion protein. A number of such vectors and suitable host systems are now available. For expression of the fusion proteins, the coding sequence will be provided with operably linked start and stop codons, promoter and terminator regions and usually a replication system to provide an expression vector for expression in the desired cellular host. For example, promoter sequences compatible with bacterial hosts are provided in plasmids containing convenient restriction sites for insertion of the desired coding sequence. The resulting expression vectors are transformed into suitable bacterial hosts. Of course, yeast or mammalian cell hosts may also be used, employing suitable vectors and control sequences.

Proteinaceous components of vaccines are preferably substantially free of other naturally occurring host cell proteins and fragments thereof. In some embodiments peptides or polypeptides can be synthetically conjugated to native fragments or particles. The polypeptides or peptides can be a variety of lengths, either in their neutral (uncharged) forms or in forms which are salts, and either free of modifications such as glycosylation, side chain oxidation, or phosphorylation or containing these-modifications, subject to the condition that the modification not destroy the biological activity of the polypeptides as herein described.

Ideally, effective vaccines are safe, not themselves causing or exacerbating injury or death and give long-term (usually years) of protection against the disease state they treat by inducing either specific cytotoxic T-cells or specific antibodies or both. As the foregoing discussion illustrates, the compositions used as vaccines historically are often complex. In the case of vaccines such as those containing intact cells or viruses, their specific constituents are often not fully defined at a molecular level. General concerns about vaccine safety and the related desire to precisely modulate immune responses (specifically, to address the concern that inappropriately designed vaccines might trigger deliterious immune responses) have led to design of highly defined vaccine entities over the last few years. In one embodiment, such highly defined vaccine compositions contain only those immunogenic peptide epitopes to which the designers intend to direct cellular or humoral immune responses, and sometimes T-helper epitopes intended to augment specifically generated immune responses. One approach to highly defined vaccines would be administration of specific peptide epitopes either directly to the patient, or to a cell product taken from the patient (such a cell product or immune effector molecules or cells derived from it can then be administered to the patient, where the effector would elicit a desired therapeutic effect).

Vaccine Administration

For pharmaceutical compositions, vaccines are administered to an individual already suffering from cancer, infected with a virus or parasite of interest, or otherwise affected by a condition that can be addressed by an immune response. Those in the incubation phase or the acute phase of infection can be treated with vaccines separately or in conjunction with other treatments, as appropriate. In therapeutic applications, compositions are administered to a patient in an amount sufficient to elicit an effective CTL response to the antigen of interest and to cure or at least partially arrest symptoms and/or complications. An amount adequate to accomplish this is defined as “therapeutically effective dose.” Amounts effective for this use will depend on, e.g., the composition, the manner of administration, the stage and severity of the disease being treated, the weight and general state of health of the patient, and the judgment of the prescribing physician, but generally range for the initial immunization (that is for therapeutic or prophylactic administration) from about 1.0 μg about 5000 μg of vaccine for a 70 kg patient, followed by boosting dosages of from about 1.0 μg to about 1000 μg of vaccine pursuant to a boosting regimen over weeks to months depending upon the patient's response and condition by measuring specific CTL activity in the patient's blood. It must be kept in mind that vaccine compositions of the present invention may, in one embodiment, be employed in serious disease states, that is, life-threatening or potentially life threatening situations. In such cases, in view of the minimization of extraneous substances and the relative nontoxic nature of the peptides, it is possible and may be felt desirable by the treating physician to administer substantial excesses of these compositions.

For therapeutic use, administration should begin at the first sign of viral infection or the detection or surgical removal of tumors or shortly after diagnosis in the case of acute infection or other condition amenable to treatment by vaccination. This is followed by boosting doses until at least symptoms are substantially abated and for a period thereafter. In chronic infection, loading doses followed by boosting doses may be required.

Treatment of an infected individual with the compositions designed using the invention may hasten resolution of the infection or pathological condition in acutely affected individuals. For those individuals susceptible (or predisposed) to developing chronic infections or conditions the compositions are particularly useful in methods for preventing the evolution from acute to chronic infection. Where the susceptible individuals are identified prior to or during infection or development of the pathological condition, for instance, as described herein, the composition can be targeted to them, minimizing need for administration to a larger population.

Vaccine compositions can also be used for the treatment of chronic infection and to stimulate the immune system to eliminate virus-infected cells in carriers. It is important to provide an amount of immuno-potentiating antigen in a formulation and mode of administration sufficient to effectively stimulate a cytotoxic T cell response. Thus, for treatment of chronic infection, a representative dose is in the range of about 1.0 μg to about 5000 μg, preferably about 5 μg to 1000 μg for a 70 kg patient per dose. Immunizing doses followed by boosting doses at established intervals, e.g., from one to four weeks, may be required, possibly for a prolonged period of time to effectively immunize an individual. In the case of chronic infection, administration should continue until at least clinical symptoms or laboratory tests indicate that the viral infection or pathological condition has been eliminated or substantially abated and for a period thereafter.

The pharmaceutical compositions for therapeutic treatment are intended for parenteral, topical, oral or local administration. Preferably, the pharmaceutical compositions are administered parenterally, e.g., intravenously, subcutaneously, intradermally, or intramuscularly. Thus, the invention provides compositions for parenteral administration which comprise a solution of the vaccine dissolved or suspended in an acceptable carrier, preferably an aqueous carrier. A variety of aqueous carriers may be used, e.g., water, bacteriostatic water, buffered water, 0.9% saline, 0.3% glycine, hyaluronic acid and the like. These compositions may be sterilized by conventional, well known sterilization techniques, or may be sterile filtered. The resulting aqueous solutions may be packaged for use as is, or lyophilized, the lyophilized preparation being combined with a sterile solution prior to administration. The compositions may contain pharmaceutically acceptable auxiliary substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, etc.

The concentration of CTL stimulatory vaccines in the pharmaceutical formulations can vary widely, i.e., from less than about 0.1%, usually at or at least about 2% to as much as 20% to 50% or more by weight, and will be selected primarily by fluid volumes, viscosities, etc., in accordance with the particular mode of administration selected.

Vaccines may also be administered via liposomes, which target them to particular cells or tissue, such as lymphoid tissue. Liposomes are also useful in increasing the half-life of the vaccine. Liposomes include emulsions, foams, micelles, insoluble monolayers, liquid crystals, phospholipid dispersions, lamellar layers and the like. In these preparations the vaccine to be delivered is incorporated as part of a liposome, alone or in conjunction with a molecule which binds to, e.g., a receptor prevalent among lymphoid cells, such as monoclonal antibodies which bind to the CD45 antigen, or with other therapeutic or immunogenic compositions. Thus, liposomes filled with a desired vaccine can be directed to the site of lymphoid cells, where the liposomes then deliver the selected therapeutic/immunogen compositions. Liposomes for use in the invention are formed from standard vesicle-forming lipids, which generally include neutral and negatively charged phospholipids and a sterol, such as cholesterol. The selection of lipids is generally guided by consideration of, e.g., liposome size, acid lability and stability of the liposomes in the blood stream. A variety of methods are available for preparing liposomes, as described in, e.g., Szoka et al., Ann. Rev. Biophys. Bioeng. 9:467 (1980), U.S. Pat. Nos. 4,235,871, 4,501,728, 4,837,028, and 5,019,369, incorporated herein by reference.

For targeting to the immune cells, a ligand to be incorporated into the liposome can include, e.g., antibodies or fragments thereof specific for cell surface determinants of the desired immune system cells. A liposome suspension containing a vaccine may be administered intravenously, locally, topically, etc. in a dose which varies according to, inter alia, the manner of administration, the vaccine being delivered, and the stage of the disease being treated.

For solid compositions, conventional nontoxic solid carriers may be used which include, for example, pharmaceutical grades of mannitol, lactose, starch, magnesium stearate, sodium saccharin, talcum, cellulose, glucose, sucrose, magnesium carbonate, and the like. For oral administration, a pharmaceutically acceptable nontoxic composition is formed by incorporating any of the normally employed excipients, such as those carriers previously listed, and generally 10-95% of active ingredient, that is, a polypeptide designed by the method of the invention, and more preferably at a concentration of 25%-75%.

For aerosol administration, vaccines are preferably supplied in finely divided form along with a surfactant and propellant. Typical percentages of peptides are 0.01 %-20% by weight, preferably 1%-10%. The surfactant must, of course, be nontoxic, and preferably soluble in the propellant. Representative of such agents are the esters or partial esters of fatty acids containing from 6 to 22 carbon atoms, such as caproic, octanoic, lauric, palmitic, stearic, linoleic, linolenic, olesteric and oleic acids with an aliphatic polyhydric alcohol or its cyclic anhydride. Mixed esters, such as mixed or natural glycerides may be employed. The surfactant may constitute 0.1%-20% by weight of the composition, preferably 0.25-5%. The balance of the composition is ordinarily propellant. A carrier can also be included, as desired, as with, e.g., lecithin for intranasal delivery.

In another aspect the present invention is directed to vaccines which contain as an active ingredient an immunogenically effective amount of an immunogenic polypeptide designed as described herein. The polypeptide(s) may be introduced into a host, including humans, linked to its own carrier or as a homopolymer or heteropolymer of active peptide units. Such a polymer has the advantage of increased immunological reaction and, where different polypeptides are used to make up the polymer, the additional ability to induce antibodies and/or CTLs that react with different antigenic determinants of the virus or tumor cells. Useful carriers are well known in the art, and include, e.g., thyroglobulin, albumins such as bovine serum albumin, tetanus toxoid, polyamino acids such as poly(lysine:glutamic acid), hepatitis B virus core protein, hepatitis B virus recombinant vaccine and the like. The vaccines can also contain a physiologically tolerable (acceptable) diluent such as water, phosphate buffered saline, or saline, and further typically include an adjuvant. Adjuvants such as incomplete Freund's adjuvant, aluminum phosphate, aluminum hydroxide, or alum are materials well known in the art. And, as mentioned above, CTL responses can be primed by conjugating peptides of the invention to lipids, such as P₃ CSS. Upon immunization with a peptide composition as described herein, via injection, aerosol, oral, transdermal or other route, the immune system of the host responds to the vaccine by producing large amounts of CTLs specific for the desired antigen, and the host becomes at least partially immune to later infection, or resistant to developing chronic infection.

Vaccine compositions designed using the invention are administered to a patient susceptible to or otherwise at risk of viral or parasitic infection, cancer or other condition amenable to immunotherapeutic intervention to elicit an immune response against the antigen and thus enhance the patient's own immune response capabilities. Such an amount is defined to be an “immunogenically effective dose.” In this use, the precise amounts again depend on the patient's state of health and weight, the mode of administration, the nature of the formulation, etc., but generally range from about 1.0 μg to about 5000 μg per 70 kilogram patient, more commonly from about 10 μg to about 500 μg per 70 kg of body weight.

In some instances it may be desirable to combine vaccines of the invention with vaccines which induce neutralizing antibody responses to pathogens of interest, particularly to viral envelope antigens.

For therapeutic or immunization purposes, the peptides of the invention can also be expressed by attenuated viral hosts, such as vaccinia or fowlpox. This approach involves the use of vaccinia virus as a vector to express nucleotide sequences that encode the peptides of the invention. Upon introduction into an acutely or chronically infected host or into a noninfected host, the recombinant vaccinia virus expresses the immunogenic peptide or polypeptide, and thereby elicits a host CTL response. Vaccinia vectors and methods useful in immunization protocols are described in, e.g., U.S. Pat. No. 4,722,848, incorporated herein by reference. Another vector is BCG (Bacille Calmette Guerin). BCG vectors are described in Stover et al. (Nature 351:456-460 (1991)) Which is incorporated herein by reference. A wide variety of other vectors useful for therapeutic administration or immunization of the polypeptides of the invention, e.g., Salmonella typhi vectors and the like, will be apparent to those skilled in the art from the description herein.

Antigenic peptides or polypeptides may be used to elicit CTL ex vivo, as well. The resulting CTL, can be used to treat chronic infections (viral or bacterial) or tumors in patients that do not respond to other conventional forms of therapy, or will not respond to a peptide vaccine approach of therapy. Ex vivo CTL responses to a particular pathogen (infectious agent or tumor antigen) are induced by incubating in tissue culture the patient's CTL precursor cells (CTLp) together with a source of antigen-presenting cells (APC) and the appropriate immunogenic polypeptide. After an appropriate incubation time (typically 1-4 weeks), in which the CTLp are activated and mature and expand into effector CTL, the cells are infused back into the patient, where they will destroy their specific target cell (an infected cell or a tumor cell). In order to optimize the in vitro conditions for the generation of specific cytotoxic T cells, the culture of stimulator cells is maintained in an appropriate serum-free medium.

Prior to incubation of the stimulator cells with the cells to be activated, e.g., precursor CD8+ cells, an amount of antigenic polypeptide is added to the stimulator cell culture, of sufficient quantity to become loaded onto the human Class I MHC molecules to be expressed on the surface of the stimulator cells. In the present invention, a sufficient amount of peptide is an amount that will allow about 200, and preferably 200 or more, human Class I MHC molecules loaded with peptide epitopes to be expressed on the surface of each stimulator cell. Preferably, the stimulator cells are incubated with >20 μg/ml polypeptide.

Resting or precursor CD8+ cells are then incubated in culture with the appropriate stimulator cells for a time period sufficient to activate the CD8+ cells. Preferably, the CD8+ cells are activated in an antigen-specific manner. The ratio of resting or precursor CD8+ (effector) cells to stimulator cells may vary from individual to individual and may further depend upon variables such as the amenability of an individual's lymphocytes to culturing conditions and the nature and severity of the disease condition or other condition for which the within-described treatment modality is used. Preferably, however, the lymphocyte:stimulator cell ratio is in the range of about 30:1 to 300: 1. The effector/stimulator culture may be maintained for as long a time as is necessary to stimulate a therapeutically useable or effective number of CD8+ cells.

The induction of CTL in vitro requires the specific recognition of peptides that are bound to allele specific MHC class I molecules on APC. The number of specific MHC/peptide complexes per APC is crucial for the stimulation of CTL, particularly in primary immune responses. While small amounts of peptide/MHC complexes per cell are sufficient to render a cell susceptible to lysis by CTL, or to stimulate a secondary CTL response, the successful activation of a CTL precursor (pCTL) during primary response requires a significantly higher number of MHC/peptide complexes. Peptide loading of empty major histocompatability complex molecules on cells allows the induction of primary cytotoxic T lymphocyte responses. Peptide loading of empty major histocompatability complex molecules on cells enables the induction of primary cytotoxic T lymphocyte responses.

Since mutant cell lines do not exist for every human MHC allele, it is advantageous to use a technique to remove endogenous MHC-associated peptides:from the surface of APC, followed by loading the resulting empty MHC molecules with the immunogenic peptides of interest. The use of non-transformed (non-tumorigenic), non-infected cells, and preferably, autologous cells of patients as APC is desirable for the design of CTL induction protocols directed towards development of ex vivo CTL therapies.

A stable MHC class I molecule is a trimeric complex formed of the following elements: 1) a peptide usually of 8-11 residues, 2) a transmembrane heavy polymorphic protein chain which bears the peptide-binding site in its alpha-1 and alpha-2 domains, and 3) a non-covalently associated non-polymorphic light chain, beta-2 microglobulin. Removing the bound peptides and/or dissociating the beta-2 microglobulin from the complex renders the MHC class I molecules nonfunctional and unstable, resulting in rapid degradation. All MHC class I molecules isolated from PBMCs have endogenous peptides bound to them. Therefore, the first step is to remove all endogenous peptides bound to MHC class I molecules on the APC without causing their degradation before exogenous peptides can be added to them.

Two possible ways to free up MHC class I molecules of bound peptides include lowering the culture temperature from 37° C. to 26° C. overnight to destablize beta-2 microglobulin and stripping the endogenous peptides from the cell using a mild acid treatment. The methods release previously bound peptides into the extracellular environment allowing new exogenous peptides to bind to the empty class I molecules. The cold-temperature incubation method enables exogenous peptides to bind efficiently to the MHC complex, but requires an overnight incubation at 26° C. which may slow the cell's metabolic rate. It is also likely that cells not actively synthesizing MHC molecules (e.g., resting PBMC) would not produce high amounts of empty surface MHC molecules by the cold temperature procedure.

Harsh acid stripping involves extraction of the peptides with trifluoroacetic acid, pH 2, or acid denaturation of the immunoaffinity purified class I-peptide complexes. These methods are not feasible for CTL induction, since it is important to remove the endogenous peptides while preserving APC viability and an optimal metabolic state which is critical for antigen presentation. Mild acid solutions of pH 3 such as glycine or citrate-phosphate buffers have been used to identify endogenous peptides and to identify tumor associated T cell epitopes. The treatment is especially effective, in that only the MHC class I-peptide complexes are destabilized (and associated peptides released), while other surface antigens remain intact, including MHC class II molecules. Most importantly, treatment of cells with the mild acid solutions do not affect the cell's viability or metabolic state. The mild acid treatment is rapid since the stripping of the endogenous peptides occurs in two minutes at 4° C. and the APC is ready to perform its function after the appropriate peptides are loaded. The technique is utilized herein to make peptide-specific APCs for the generation of primary antigen-specific CTL. The resulting APC are efficient in inducing peptide-specific CD8+ CTL.

Activated CD8+ cells may be effectively separated from the stimulator cells using one of a variety of known methods. For example, monoclonal antibodies specific for the stimulator cells, for the peptides loaded onto the stimulator cells, or for the CD8+ cells (or a segment thereof) may be utilized to bind their appropriate complementary ligand. Antibody-tagged molecules may then be extracted from the stimulator-effector cell admixture via appropriate means, e.g., via well-known immunoprecipitation or immunoassay methods. Examples of such techniques are well known in the art. For instance, Lefkovits, Immunonology Methods Manual: Comprehensive Sourcebook of Techniques, Volume 2, Academic Press (1996), herein incorporated by reference in its entirety, describes standard immunological lab techniques.

Effective, cytotoxic amounts of the activated CD8+ cells can vary between in vitro and in vivo uses, as well as with the amount and type of cells that are the ultimate target of these killer cells. The amount will also vary depending on the condition of the patient and should be determined via consideration of all appropriate factors by the practitioner. Preferably, however, about 1×10⁶ to about 1×10¹², more preferably about 1×10⁸ to about 1×10¹¹, and even more preferably, about 1×10⁹ to about 1×10¹⁰ activated CD8+ cells are utilized for adult humans, compared to about 5×10⁶-5×10⁷ cells used in mice.

Preferably, as discussed above, the activated CD8+ cells are harvested from the cell culture prior to administration of the CD8+ cells to the individual being treated. It is important to note, however, that unlike other present and proposed treatment modalities, the present method uses a cell culture system that is not tumorigenic. Therefore, if complete separation of stimulator cells and activated CD8+ cells is not achieved, there is no inherent danger known to be associated with the administration of a small number of stimulator cells, whereas administration of mammalian tumor-promoting cells may be extremely hazardous.

Methods of re-introducing cellular components are known in the art and include procedures such as those exemplified in U.S. Pat. No. 4,844,893 to Honsik, et al. and U.S. Pat. No. 4,690,915 to Rosenberg. For example, administration of activated CD8+ cells via intravenous infusion is appropriate.

Vaccine and Polypeptide Design

Vaccines composed of individual peptide epitopes are highly defined, and therefore perhaps less likely to engender unexpected or undesired immune responses, but they exhibit certain limitations in their synthesis, formulation and administration. For one thing, peptides of the size of Class I epitopes (8-11 amino acids) exhibit poor pharmacokinetic properties, and are often quickly cleared from the body. Rapid clearance diminishes the ability of such peptides, when administered directly to a patient or animal, to trigger an immune response.

In an outbred population, such as the human population, the specific constituents of the T-cell repetoire of any individual are unknown. Therefore, there is a the possibility that a peptide epitope might bind Class I MHC molecules, but still fail to elicit a specific CTL response in some fraction of the population because the T-cell repetoire of that fraction of the population lacks T-cell clones that specifically recognize the presented epitope and respond to the presented epitope. Because of individual variations in outbred populations, the quality and therapeutic efficacy of immune responses to a given immunogen can vary, and responses to antigens that are therapeutically effective in one individual may not be equally efficacious in all others. Additionally, disease states themselves are not necessarily homogenous. For instance, cancers of one clinical description may present different epitopes on their Class I molecules in one individual than do clinically similar neoplasms do in other individuals. Even within an individual, cancer cells are often not homogeneous, with different cells within a single tumor presenting different epitopes on Class I molecules. Taken together, these considerations illustrate that a single immunizing epitope may not stimulate protective immunity to a given disease state for all members of an outbred population, or for all cells involved in a given disease process in any one individual. For these and other reasons, it is not prudent to depend on the immunogenicity of any single Class I peptide epitope to direct a therapeutically effective immune response to an antigen of interest. This raises another limitation inherent in peptide vaccines: multiple immunogenic peptides are desirable in vaccine compositions directed to a particular ailment. Unfortunately, each peptide component of the vaccine is a separate molecular entity, each with its own formulation, handling and regulatory issues.

Immunological dogma had held for many years that the peptide epitopes presented on Class I MHC molecules were obligately derived from polypeptides expressed in the APCs, and that extracellular polypeptides did not enter the MHC Class I processing and presentation pathway. This dogma has recently fallen. Work by Ken Rock and others have shown, that contrary to dogma, polypeptides can be taken up by APCs, processed, presented on Class I MHC, and trigger CTL responses (reviewed in S. Raychaudhuri and K. L. Rock, 1998, Nature Biotechnology 16:1025-1031). These results not only indicate that protein antigens can be effective immunogens to generate specific CTL responses, but also support a new class of vaccine entities consisting of multiple immunogenic peptide epitopes linked together into a single polypeptide chain. These so-called polyepitope vaccines offer some of the advantages of peptide vaccines in that they are specifically defined molecular entities, and additionally eliminate the complexity associated with therapeutics that, like many peptide vaccines, are comprised of multiple molecular entities. They accomplish this latter advantage by linking multiple immunogenic peptides into a single polypeptide chain.

These advantages notwithstanding, polyepitope vaccines can have disadvantages of their own, key among them is that linking together individual epitopes can produce novel epitopes spanning the C-terminus of the upstream epitope and the N-terminus of the downstream epitope. Because of their chimeric nature, such epitopes, referred to as “junctional epitopes” typically do not correspond to an antigen associated with the disease process under treatment, but their chance cross-reactivity to other host antigens cannot be ruled out. This opens the possibility that junctional epitopes could trigger undesired autoimmune responses. For this reason alone, it is desirable that junctional epitopes be excluded from designed vaccines whenever possible. The present invention provides a method to design vaccines that exclude junctional epitopes through systematic consideration of the binding motifs for various Class I MHC alleles. Clearly, when existing motifs are modified in light of new data, or when new motifs become available, those modified/nascent motifs will be fully amenable to consideration for polypeptide design using the method described herein.

The present design method is applicable to design of polypeptides other than vaccines, in many cases the designed polypeptides may have critical properties in addition to their immunological properties, that are also determined by their amino acid sequences, and that must be incorporated in the final designed polypeptide. In these cases, the epitope manipulation activities are as described for vaccines.

For instance, if a polypeptide must exhibit both an enzymatic activity as well as a defined epitope composition, application of the instant invention provides the protein engineer with a comprehensive menu of variant polypeptides exhibiting the targeted epitope distribution. As illustrated in the examples which follow, the number of variants of a given polypeptide sequence exibiting any given targeted epitope distribution is vast (sometimes in excess of 10²³ variants of a single 100 amino acid polyepitope sequence, for instance), so the probability that a protein with the desired epitope distribution and biological activity will be available from a collection of sequences designed using the instant invention is very high.

### EXAMPLES

Selection of MHC Alleles for Consideration and their Binding Motifs

As we have seen, the Class I MHC binding motifs (and therefore the peptide epitopes) that will be recognized by the MHC Class I molecules of any individual are a function of the MHC Class I binding proteins expressed on the cell surfaces of that individual and encoded by the MHC (HLA) locus(i) of the individual. We have also seen that the distribution of different MHC Class I alleles within a given target population is related to racial and ethnic composition of the target population (see again; Table 1). Therefore, in the instant invention, Class I binding motifs that will be considered to design polypeptides using the instant invention are chosen, in one embodiment, in consideration of the distribution of MHC Class I alleles in the intended target population for the therapeutic under design.

The intended target population could be a group limited to a single cell or a single individual, and the MHC Class I binding motifs that would be considered to design a therapeutic for that population could be chosen based on the complement of MHC Class I molecules of the target cells or individual. The identity of these MHC Class I molecules can be determined empirically by typing methods well known to those skilled in the art, or the probable MHC complement of the individual might be infeffed based on the race and ethuicity of the individual and the known frequencies of the different MHC alleles within those ethnic or racial groups (see Table 1).

At the other extreme, the intended target population might be much broader than one or a few individuals, potentially as large as a nation or a population of known ethnic composition. Alternatively, the target population could consist of individuals who are, for any reason, suspected to be at risk for a disease state that can be addressed by vaccination. In one embodiment, the binding motifs that will be used to design the polypeptide therapeutic are chosen in consideration of the ethnic and racial demographics of the target population, and the known frequencies of different MHC Class I alleles occurring in the relevant ethnic and racial groups. In one embodiment, sets of binding motifs to be considered in the instant polypeptide design invention are chosen such that the known frequencies of Class I MHC alleles (see Table 1) would dictate that the majority of members of the target population would have one or more of the MHC Class I alleles whose binding motifs are considered in design of the polypeptide.

In one embodiment, we will design a polyepitope vaccine for use in the United States. The American population is majority Caucasian, and inspection of Table 1 demonstrates that the majority of the caucasian population possesses at least one of the following MHC Class I alleles: A2 (including the A2.1, A2.2, A2.3 subtypes), A3, B7, A1b, A24, B27, B44.

Having identified the MHC Class I alleles prevalent in the target population, the corresponding binding motifs are of interest to use in the instant design invention. These binding motifs can be determined empirically by means known to those skilled in the art and/or described in the current application, or can be taken from literature sources or other scientific communications. For each MHC Class I allele under consideration, the position and spacing of the intermediate, N—, and C-terminal anchor residues, as well as the set of amino acids that are dictated by each motif at each of the anchor positions will be considered using the instant invention to design a polypeptide. We have found that tables which specify the amino acid position (amino acid positions 1-10) of the epitope along one dimension and the MHC subtype along the other dimension are a useful way to summarize the needed information. Entered into the tables following each allele and under the amino acid position are the set of amino acids allowable as anchor residues. For this example, those binding motifs are specified in Table 4. We will consider only binding motifs of 8-10 amino acids in length in this illustrative example, because such epitopes are far more common than epitopes of 11 amino acids in length. However, the instant invention and our claims to it are not limited to consideration of epitopes of 8-10 amino acids. It will be apparent to the artisan that the design method is applicable to epitopes and MHC Class I binding motifs of other lengths.

TABLE 4

MHC Class I complex Binding Motif Anchor Summary.

| | |
|---|---|
| MHC Class I | Epitope Amino acid position |
| Alleles | 1 2 3 4 5 6 7 8 9/10 |

| | |
|---|---|
| A₂ | LM VIL |
| A₃ | LMVT KR |
| B₇ | P LIMVF |
| A_{1b} | TS Y |
| A_{1c} | DE Y |
| A₂₄ | Y F |
| B₂₇ | R FLRK |
| B₄₄ | E FY |

The designation “A2” incorporates binding motifs for the related MHC alleles A2.1, A2.2 and A2.3. In one embodiment, for each allele, the known anchor residues and their positions in Class I epitopes are taken from the open literature. For convenience, these are summarized above in tabular form.

Selection of Epitopes for Inclusion in a Vaccine

In one embodiment, epitopes for inclusion in a vaccine are chosen for their relevency to a disease state, and there is a presumption by the vaccine designer that immune responses to the chosen epitopes have therapeutic value. This presumption can be supported by a variety of information from a variety of sources. The instant invention is not directed to identification of or verification of the efficacy of any given epitope as a therapeutic. It is directed instead to design of polypeptides that exhibit controlled distributions of epitopes. Nonetheless, amino acid sequences to be considered using the method are needed to exemplify the method, and in one embodiment such sequences are epitopes that might be included in a vaccine.

In one embodiment, the invention is used to design a vaccine intended to treat cancer, and epitopes of cancer-associated antigens are incorporated into a designed polyepitope vaccine. In one embodiment, such epitopes are listed in Table 5. The epitopes of Table 5 are derived from cancer-associated antigens, with the exception of epitope 2, which is a T-helper epitope.

TABLE 5

Class I epitopes for inclusion in a
polyepitope vaccine. Epitopes are listed
here using the standard one letter amino
acid code. In each epitope, The leftmost
residue is the N-terminal amino acid, and
the rightmost is the C-terminal amino acid.

1. KLCPVQLWV
2. AKFVAAWTLKAAA
3. KVAELVHFL
4. VVLGVVFGI
5. YLQLVFGIEV
6. IMIGVLVGV
7. YLSGANLNV
8. RLLQETELV
9. SMPPPGTRV

Identification of Junctional Epitopes and their Manipulation in Consideration of Spacing Requirements of MHC Class I Binding Motifs

In one embodiment of the current invention, the method can be used to design vaccines whose distribution of MHC Class I epitopes is controlled by the designer, particularly for a vaccine comprised of MHC Class I epitopes. However, it should be readily apparent that the neither the method nor our claim to it are limited to this example. The artisan will recognize that the method of the instant invention, though demonstrated here to design polyepitope vaccines with controlled distributions of epitopes, is equally applicable to design of variants of any parent polypeptide sequence such that those variants exhibit a distribution of epitopes selected by the designer.

As described throughout the current application, epitopes conform to specific Class I MHC binding motifs. Thus, when two epitopes are abutted in a designed polyepitope, if there are amino acids suitable as N-end anchors for epitopes for a given MHC Class I allele within one of the vaccine epitopes, and there are the corresponding C-end anchors in the next (more C-terminal) vaccine epitope, and if the anchor residues are spaced appropriately, an epitope, specifically referred to as a junctional epitope will result. Junctional epitopes do not correspond to a naturally occuring antigen: they are hybrid structures containing components from multiple natural epitopes. Nonetheless, their ability to bind Class I MHC molecules is a function of conformation to the binding motif, And so junctional epitopes can potentially compete for binding of the MHC:Class I with the desired vaccine epitopes, can thereby suppress immune responses to the desired epitopes, and are therefore themselves undesirable. This particular example will focus on design of a single epitope-epitope junction that contains a predetermined number of junctional epitopes (in this case, 0 junctionals). Obviously, to complete design of an entire polyepitope, the process must be repeated for each adjacent pair of epitopes overlapping or non-overlapping in the polyepitope.

In one embodiment, the initial step is to identify undesired epitopes occurring across vaccine epitope-vaccine epitope junctions by their congruence to known MHC binding motifs. Those undesired junctional epitopes can be eliminated by inserting or deleting amino acids between the vaccine epitopes such that spacing between the anchors of the junctional epitopes is modified so as to not satisfy the relevant Class I MHC binding motifs.

It is also possible to eliminate junctionals by deleting anchor residues or substituting anchor positions with amino acids that are not allowed by the MHC Class I binding motif in question. In one embodiment, the purpose of the vaccine is to trigger immune responses to the vaccine epitopes intentionally included in its composition, and by definition, the anchors of junctionals lie within vaccine epitopes such that modifying junctional epitope positions would result in modifying vaccine epitopes. Consequently, controlling spacing between vaccines epitopes will be used to eliminate junctional epitopes in the explified embodiment, rather than substitution or deletion of anchor residues or deletion of residues between anchor residues. However, avoidance of anchor residues in amino acids inserted between vaccine epitopes is practiced in one embodiment of the invention (vaccine design). If amino acids allowable as intermediate, N— or C-terminal anchor residues are introduced between vaccine epitopes, and there are corresponding intermediate, C— or N-terminal anchors in the vaccine epitopes that satisfy the corresponding MHC Class I binding motifs, new epitopes will be generated. Like junctional epitopes, these new epitopes do not correspond to epitopes that are relevant to the disease state the designed vaccine is intended to treat. As such, these new epitopes are as undesirable as junctional epitopes for much the same reasons. The amino acids inserted to eliminate junctional epitopes are selected such that they do not introduce new anchor residues spaced appropriately from residues of the vaccine epitopes that flank them such that a new, undesired epitope is created de novo.

In one embodiment, the two vaccine epitopes intended to abut each other are examined to identify N-terminal anchor residues for motifs corresponding to the MHC Class I binding motifs of interest occurring in the N-terminal of the vaccine epitopes and C-terminal anchors for motifs corresponding to the MHC Class I binding motifs of interest occurring in the C-terminal of the vaccine epitopes. In another embodiment, the two vaccine epitopes intended to abut each other are examined to identify intermediate anchor residues for motifs corresponding to the MHC class I binding motifs of interest occurring in the intermediate anchor residue of the vaccine epitopes. The specific linker length needed to eliminate junctional epitopes across any vaccine epitope pair is determined by the distribution of N end and C end anchors in the vaccine epitopes that flank their juncture. Although any number of amino acids might be considered to insert between vaccine epitopes to eliminate junctional epitopes, in one embodiment it is useful to decide a range of numbers of amino acids that will be considered for insertion between vaccine epitopes in the design. This decision can be made arbitrarily by the designer, or to accommodate design features of the polypeptide beyond its epitope distribution. In one embodiment, linker length is selected such that it 1) eliminates all junctional epitopes as described, and 2) is of the shortest length that can accommodate the epitope distribution features chosen by the designer. In the instant example, no more than six amino acids will be considered for insertion between vaccine epitopes. However, longer amino acid sequences can be selected by the designer for any of a number of purposes. For example, linkers can be up to 12 amino acids in length. In another embodiment, linkers can be 6 or 8 amino acids in length. The minimum linker length can be 0, 1, 2, or 3 amino acids long. The maximum linker length possible to remove junctional epitopes is equal to the [the widest separation of N-terminal and C-terminal anchors]−1. Linkers may be longer, although the epitopes created within the linker itself will become the focus of the method.

Other than pre-selected epitope distributions, specific functional, material, biochemical or biological properties incorporated in designed polypeptides will be specific to each polypeptide design project, will be idiosyncratic to the individual design efforts. The invention is directed to deriving a comprehensive menu of all polypeptides that would satisfy the design criteria for epitope distribution. Polypeptides incorporated into the list can then be screened for any other properties desired by the designer.

In one embodiment, the amino acid sequences of vaccine epitope pairs are written from left to right, with the N-terminal amino acids at the left, C-terminal amino acids at the right, with a number of blanks left between the N-terminal (upstream) vaccine epitope and the C-terminal (downstream) vaccine epitope. The number of blank spaces is the same as the number of amino acids chosen by the designer to be inserted between vaccine epitopes, in one embodiment, six amino acids, corresponding to six blank spaces between the vaccine epitopes. All amino acid residues allowable as N-terminal anchor residues for the MHC Class I binding motifs under consideration, and occuring in the upstream vaccine epitope of the pair are identified. In one embodiment, the identity of these potential anchors are indicated by the name of the MHC Class I allelic variant(s) they correspond to beneath the potential anchor residue (ie., A2, B7, etc.). All amino acid residues allowable as C-terminal anchor residues for the MHC Class I binding motifs under consideration, and occurring in the downstream vaccine epitope of the pair are identified. In one embodiment, the identity of these potential anchors are indicated by the name of the MHC Class I allelic variant(s) they correspond to beneath the potential anchor residue (ie., A2, B7, etc.). This is illustrated for the pairing of vaccine epitope 1 and vaccine epitope 2 of Table 5 in Table 6.

TABLE 6

Epitope 1 at the N-terminus, Epitope 2 at the C-terminus.
The number of blank spaces is the same as the number of amino
acids chosen by the designer to be inserted between vaccine
epitopes, in one embodiment, six amino acids, corresponding
to six blank spaces between the vaccine epitopes. All amino
acid residues allowable as N-terminal anchor residues for the
MHC Class I binding motifs under consideration, and occuring
in the upstream vaccine epitope of the pair are identified.
In one embodiment, the identity of these potential anchors
are indicated by the name of the MHC Class I allelic
variant(s) they correspond to beneath the potential anchor
residue (ie., A2, B7, etc.). All amino acid residues
allowable as C-terminal anchor residues for the MHC Class I
binding motifs under consideration, and occuring in the
downstream vaccine epitope of the pair are identified. In one
embodiment, the identity of these potential anchors are
indicated by the name of the MHC Class I allelic variant(s)
they correspond to beneath the potential anchor residue
(ie., A2, B7, etc.).

| | | | | | | | | | | | | | | | | | | | | | | | | | | | |
|---|---|---|---|---|---|---|---|---|---|---|---|---|---|---|---|---|---|---|---|---|---|---|---|---|---|---|---|
| K | L | C | P | V | Q | L | W | V | _{—} | _{—} | _{—} | _{—} | _{—} | _{—} | A | K | F | V | A | A | W | T | L | K | A | A | A |
| | A₂ | | B₇ | A₃ | | A₂ | | A₃ | | | | | | | | A₃ | B₇ | B₇ | | | | | A₂ | A₃ | | | |
| | A₃ | | | | | A₃ | | | | | | | | | | B₂₇ | A₂₄ | A₂ | | | | | B₇ | | | | |

| | |
|---|---|
| B₄₄ | B₂₇ |

B₂₇

Enumerating Junctional Epitopes as Function of the Number of Amino Acids Inserted between Vaccine Epitopes

In one embodiment, once the potential N-terminal anchor residues present in the upstream vaccine epitope and the potential C-terminal epitopes in the downstream epitope have been identified, the number of junctional epitopes that will occur across the vaccine epitopes for each number of amino acids inserted between the epitopes can be ennumerated. This exercise allows the designer to choose a number of amino acids to be inserted between vaccine epitopes that will allow the inclusion of a number of junctional epitopes in the designed polyepitope that satisfies pre-chosen design parameters. In one embodiment in polyepitope vaccine design, wherein only immune responses to the vaccine epitopes are desired, the desired number of junctional epitopes is zero.

In one embodiment, the number of junctional epitopes is determined by counting backward from the potential N-terminal anchor residues of the upstream vaccine epitopes assuming there are 0 amino acids inserted between vaccine epitopes, 1 amino acid inserted between vaccine epitopes, 2 amino acids inserted between the vaccine epitopes, and so forth up to and including the maximum number of amino acids the designer has decided to consider inserting between the vaccine epitopes. For any given number of amino acids inserted between vaccine epitopes, if a C-terminal anchor for the same MHC Class I binding motif as the N-terminal anchor occurs in the downstream epitope, and if, in consideration of the number of amino acids to be inserted between the vaccine epitopes, the spacing of the N-and C-terminal anchors satisfy the MHC Class I binding motif, one junctional epitope is scored for insertion of that number of amino acids between the vaccine epitopes. This process is repeated for each number of inserted amino acids the designer is considering. It is repeated for each potential N-terminal anchor in the upstream vaccine epitope corresponding to one of the MHC Class I binding motifs under consideration.

In one embodiment, the number of junctional epitopes is determined by counting forward from the potential C-terminal anchor residues of the downstream vaccine epitopes assuming there are 0 amino acids inserted between vaccine epitopes, I amino acid inserted between vaccine epitopes, 2 amino acids inserted between the vaccine epitopes, and so forth up to and including the maximum number of amino acids the designer has decided to consider inserting between the vaccine epitopes. For any given number of amino acids inserted between vaccine epitopes, if a N-terminal anchor for the same MHC Class I binding motif as the C-terminal anchor occurs in the upstream epitope, and if, in consideration of the number of amino acids to be inserted between the vaccine epitopes, the spacing of the N-and C-terminal anchors satisfy the MHC Class I binding motif, one junctional epitope is scored for insertion of that number of amino acids between the vaccine epitopes. This process is repeated for each number of inserted amino acids the designer is considering. It is repeated for each potential C-terminal anchor in the downstream vaccine epitope corresponding to one of the MHC Class I binding motifs under consideration.

In one embodiment, the number of junctional epitopes predicted for each number of amino acids contemplated for insertion between vaccine epitopes is tabulated. In one embodiment, the MHC Class I binding motif under consideration is listed on the vertical, and the number of junctional epitopes for each MHC Class I binding motif under consideration is listed on the horizontal. In one embodiment, only those MHC Class I binding motifs for which there is predicted to be a corresponding junctional for one or more of the number of amino acids inserted between vaccine epitopes under consideration is listed in such tables. Such a table (setting forth the number of junctional epitopes occuring between vaccine epitopes if 0, 1, 2, 3, 4, 5, or 6 amino acids are inserted between said vaccine epitopes) for vaccine epitopes I and 2 of Table 5 is shown in Table 7. In Examples 1-81, junctional epitopes per motif with respect to linker length are recorded by the N-terminal motif anchors occurring in each example.

TABLE 7

| | Amino Acids inserted between vaccine epitopes | | | | | | |
|---|---|---|---|---|---|---|---|
| MHC Class I | 0 | 1 | 2 | 3 | 4 | 5 | 6 |
| A₂ | 0 | 1 | 1 | 0 | 0 | 0 | 0 |
| A₃ | 0 | 1 | 1 | 1 | 1 | 1 | 1 |
| B₇ | 1 | 0 | 0 | 0 | 0 | 0 | 0 |
| Total | 1 | 2 | 2 | 1 | 1 | 1 | 1 |

Junctional epitopes between vaccine epitopes 1 and 2 (see Table 5) as a function of the number of amino acids inserted between vaccine epitopes.

As inspection of the Table 7 lshows, no number of amino acids up to 6, if inserted between vaccine epitopes 1 and 2 result in 0 junctional epitopes. In a polyepitope with the design parameters initially assumed (no more than 6 amino acids to be inserted between vaccine epitopes, and no junctional epitopes in the final designed polyepitope), vaccine epitopes 1 and 2 of Table 5 would not be used in the order epitope one upstream and epitope 2 immediately downstream in the designed polyepitope. However, vaccine epitopes 1 and 2 of Table 5 might be used in the order epitope 1 upstream and epitope 2 downstream if the polyepiotopes had been designed to fit other parameters (that is parameters allowing more inserted amino acids, or if more than 0 junctional epitopes were acceptable in the design).

Avoiding Generation of Epitopes De Novo by Judicious choice of Amino Acid Residues in the Designed Sequence

In one embodiment, the identities of amino acids that can be inserted into the polypeptide sequence under design without generating additional undesired epitopes can be determined at this point. The identities are determined from the N-terminal anchor residues for motifs corresponding to the MHC subtypes of interest occurring in the N-terminal vaccine epitopes, C-terminal anchors for motifs corresponding to, the MHC subtypes of interest occurring in the C-terminal vaccine epitopes, and intermediate anchors for motifs corresponding to the MHC subtypes of interest occurring in the intermediate vaccine epitopes identified above.

For each potential N-terminal anchor that occurs in the N-terminal vaccine epitope, one counts from that anchor to identify the position(s) in the inserted amino acid sequence where a corresponding C-terminal anchor must occur to generate an epitope, as dictated by the known binding motif for the MHC subtype in question. All amino acids EXCEPT those known to be C-terminal anchors for the motif in question can be inserted at that position without generating an undesired epitope. For each potential C-terminal anchor occurring in the C-terminal vaccine epitope, one counts from that anchor to identify the position(s) in the linker where a corresponding N-terminal anchor must occur to generate an epitope, as dictated by the known binding motif for the MHC subtype in question. All amino acids EXCEPT those known to be N-terminal anchors for the motif in question can be inserted at that position without generating an undesired epitope.

The result of this operation is the identification of all possible linkers within the linker size range used (in this case, 0-6 amino acids) that will give a desired number of epitopes that overlap the linker. If this process is repeated for every possible adjacent vaccine epitope pair that might be used in a polyepitope, the result of the exercise is identification of all possible polyepitope sequences that satisfy the design criterion for epitopes spanning vaccine epitope-vaccine epitope junctions. In one embodiment, in polyepitope vaccine design, only immune responses to the vaccine epitopes are desired in which the desired number of epitopes overlapping the inserted amino acids is zero.

Algorithms and Programmed Computers for Performing the Method

In one embodiment, the method can be performed using a computer algorithm. In another embodiment, one and a second polypeptide sequences are input into a computer, MHC binding motifs are designated, and sequence algorithm program parameters are designated. The sequence comparison algorithm then calculates the number and identity of amino acids needed in a linker needed to avoid junctional epitopes, based on the designated program parameters (the MHC binding motifs). In yet another embodiment, a polypeptide sequence is inputed into a computer, MHC binding motifs are designated, and sequence algorithm program parameters are designated. The sequence comparison algorithm then calculates changes in the identity of amino acids that will avoid the designated parameters.

The algorithm can be implemented in hardware or software, or a combination of both (e.g., programmable logic arrays or digital signal processors). In particular, various general purpose machines may be used with programs written in accordance with the teachings herein, or it may be more convenient to construct more specialized apparatus to perform the operations. However, preferably, the algorithm is implemented in one or more computer programs executing on programmable systems each comprising at least one processor, at least one data storage system (including volatile and non-volatile memory and/or storage elements), at least one input device, and at least one output device. The program code is executed on the processors to perform the functions described herein.

Each such program may be implemented in any desired computer language (including machine, assembly, high level procedural, or object oriented programming languages) to communicate with a computer system. In any case, the language may be a compiled or interpreted language.

Each such computer program is preferably stored on a storage media or device (e.g., ROM, CD-ROM, or magnetic or optical media) readable by a general or special purpose programmable computer, for configuring and operating the computer when the storage media or device is read by the computer to perform the procedures described herein. The system may also be considered to be implemented as a computer-readable storage medium, configured with a computer program, where the storage medium so configured causes a computer to operate in a specific and predefined manner to perform the functions described herein.

As used herein, programmed computer means hardware or software contining the algorithm.

In one embodiment, those functions are pattern-matching an MHC binding motif to a symbolic polypeptide, and changing amino acids in the polypeptide to alter the pattern-match. In another embodiment, those functions are pattern-matching an MHC binding motif to a symbolic polypeptide, and adding or subtracting amino acids in the polypeptide to alter the pattern-match.

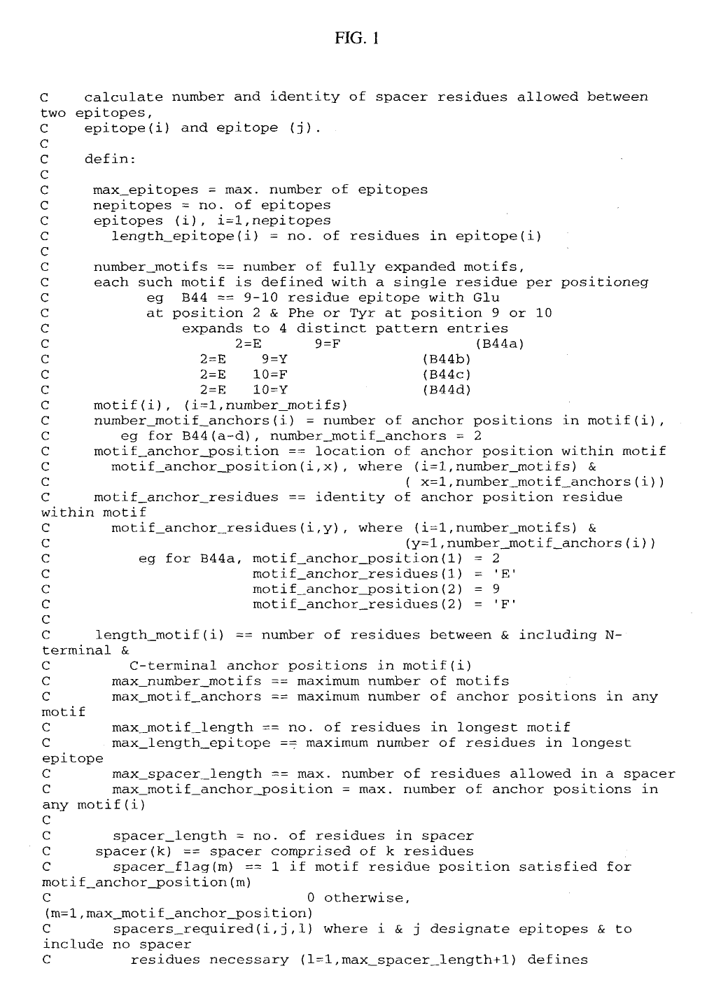

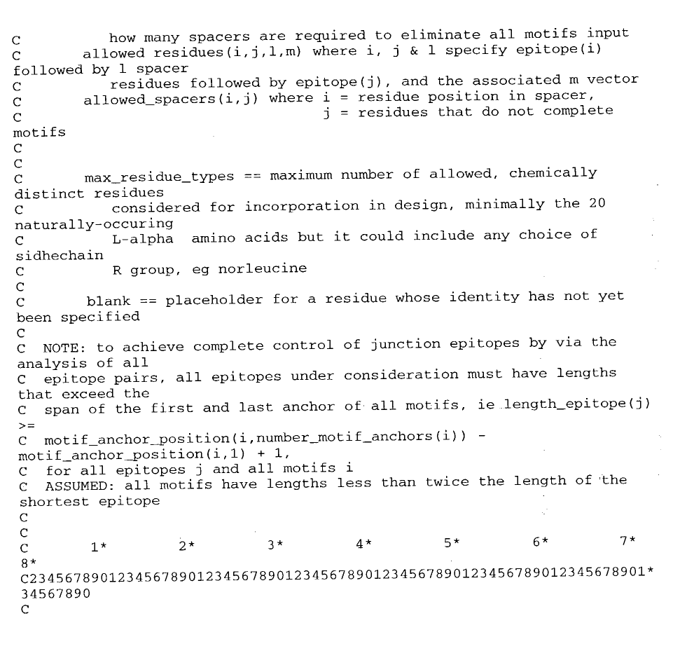

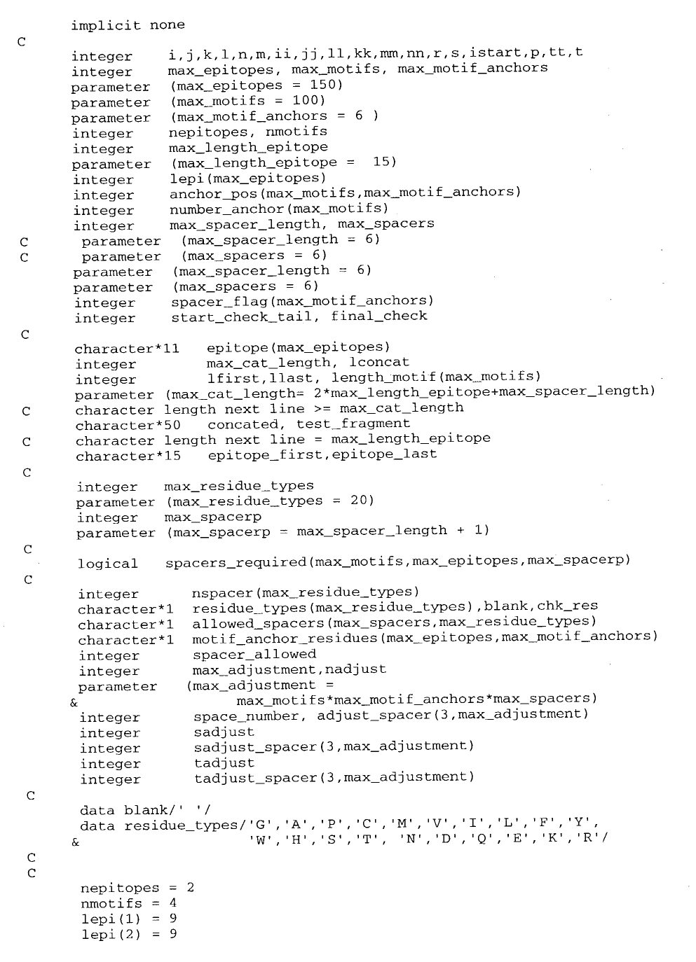

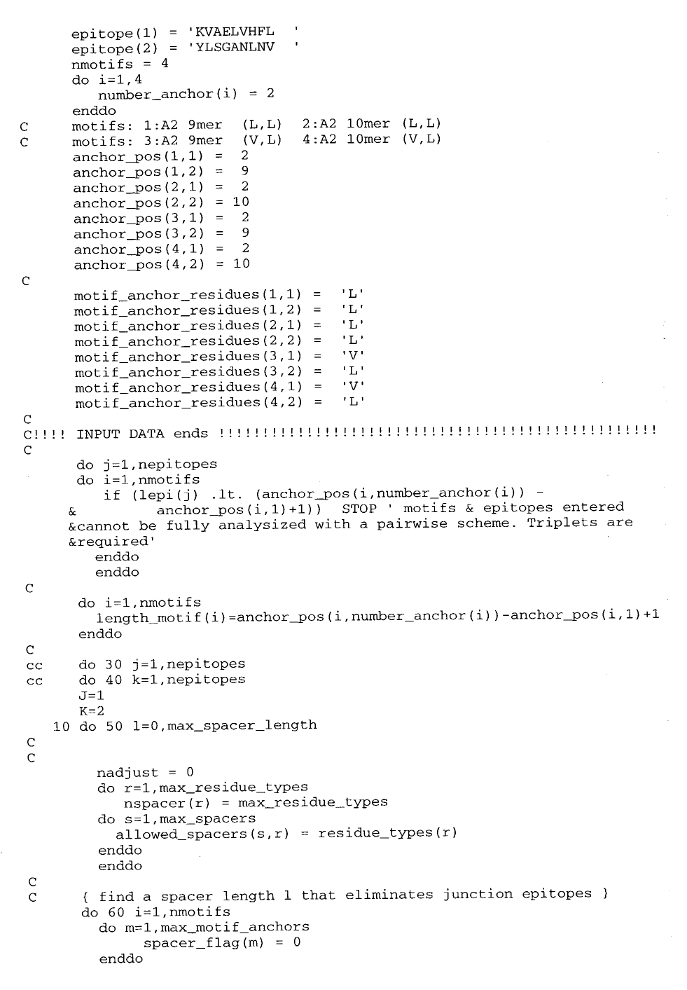

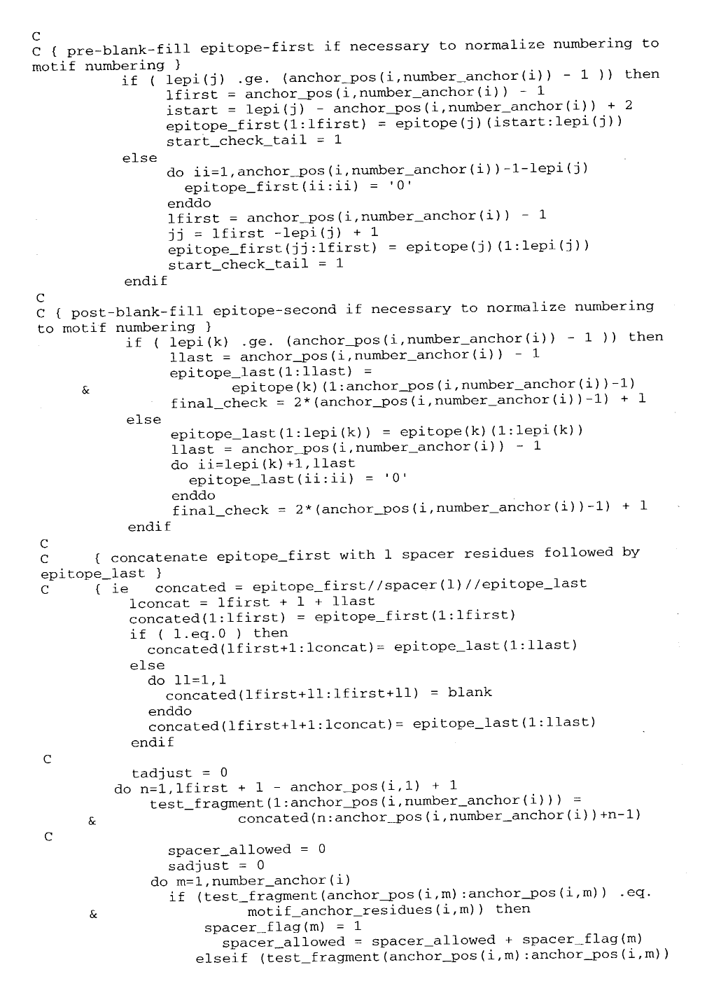

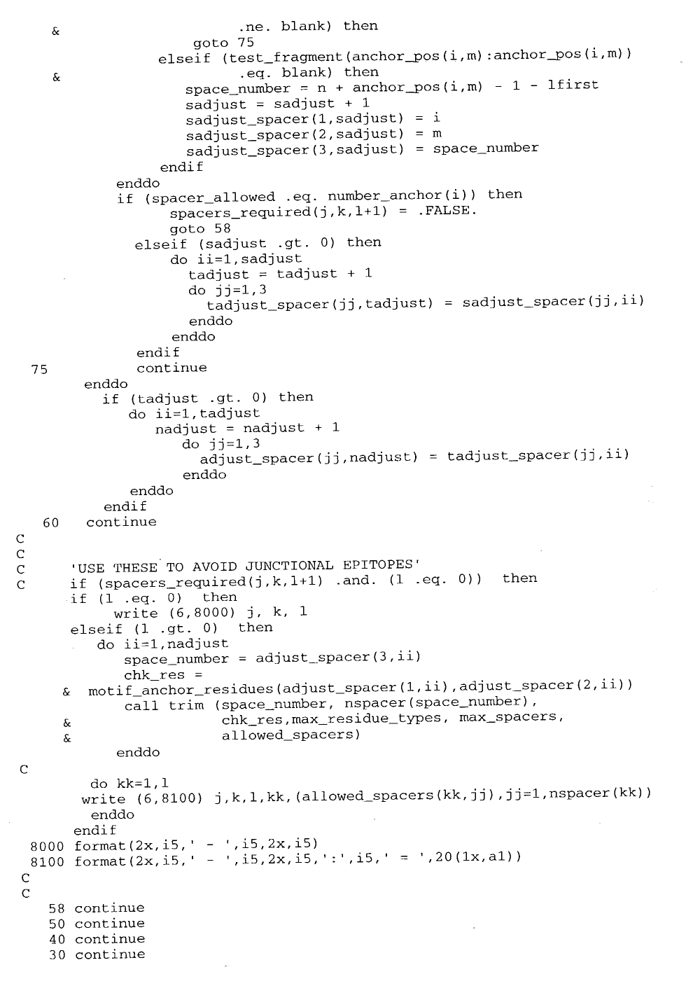

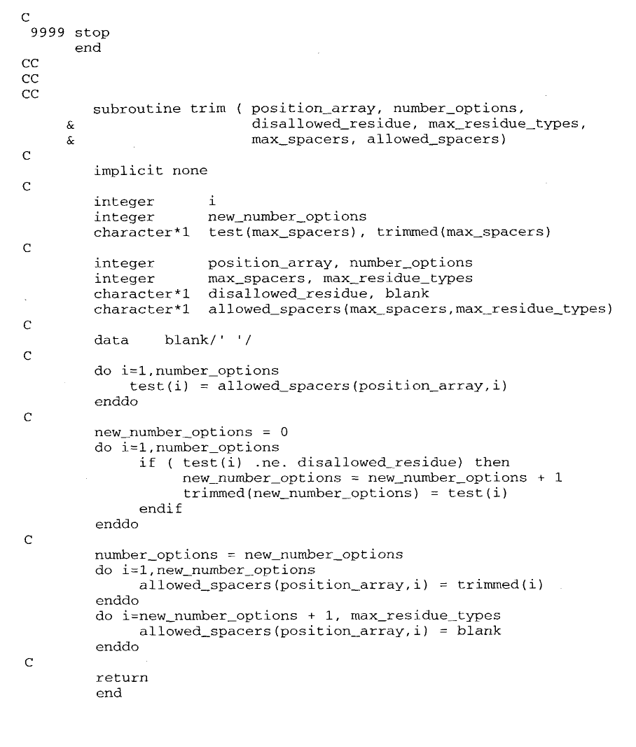

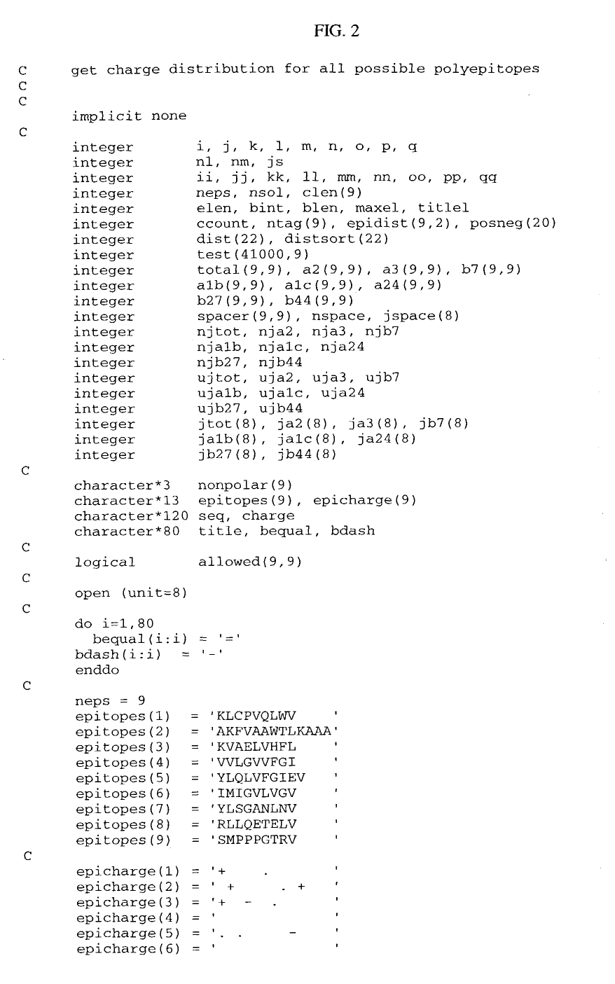

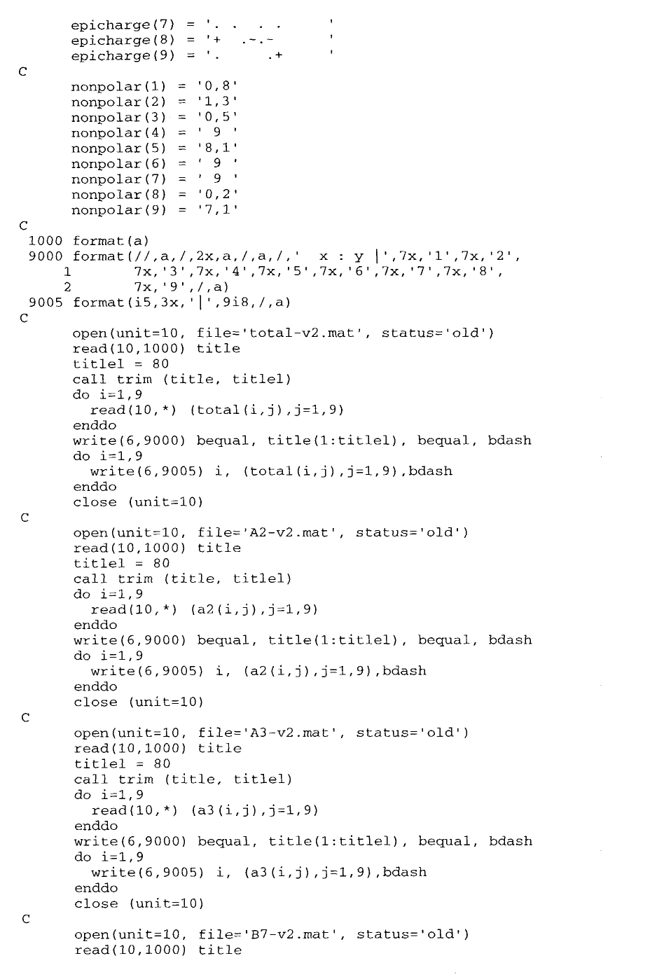

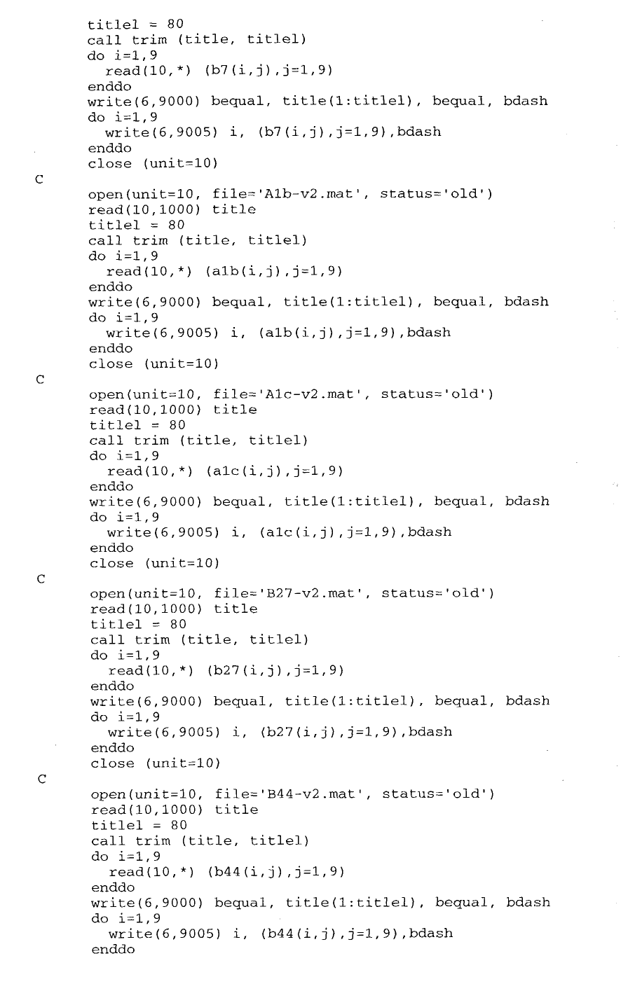

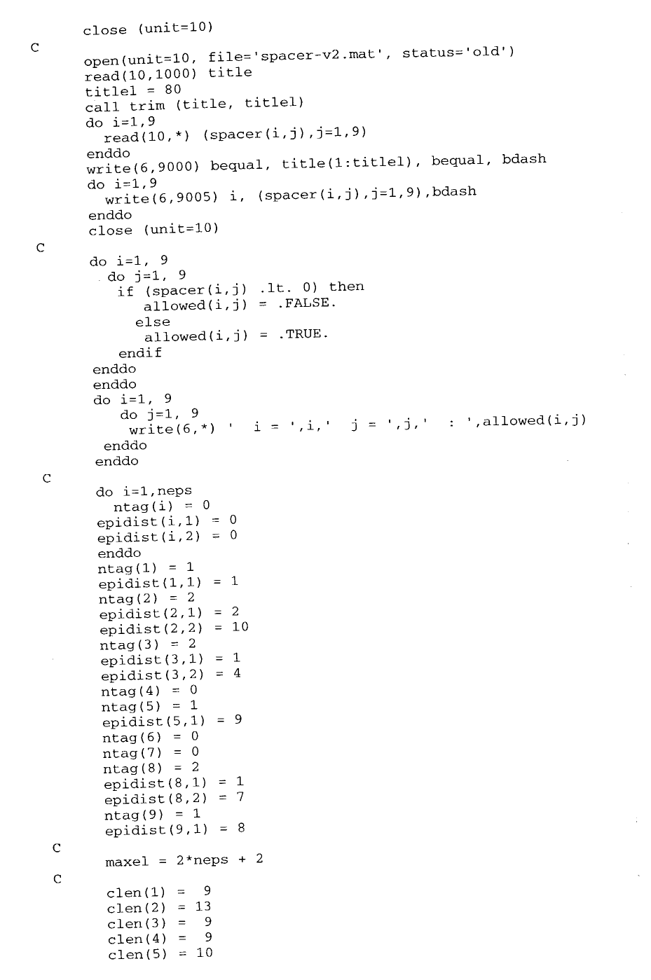

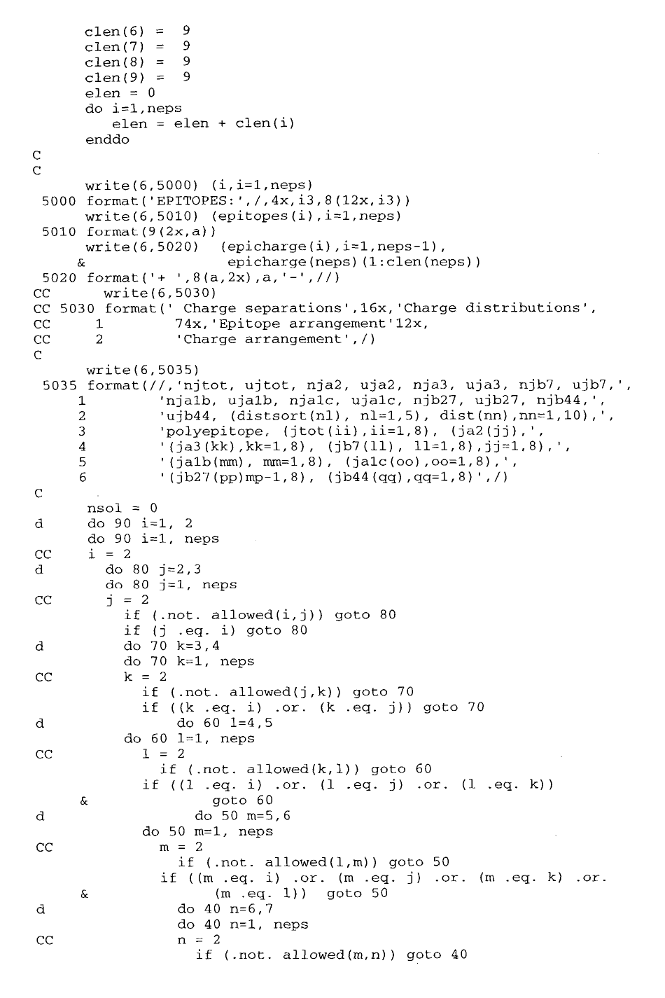

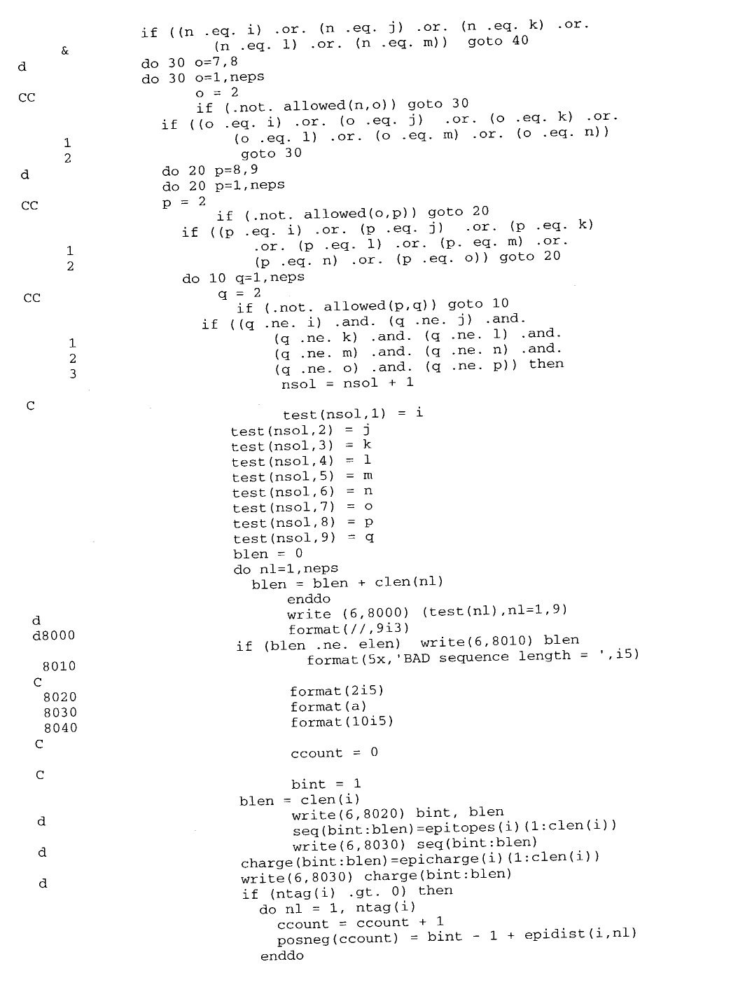

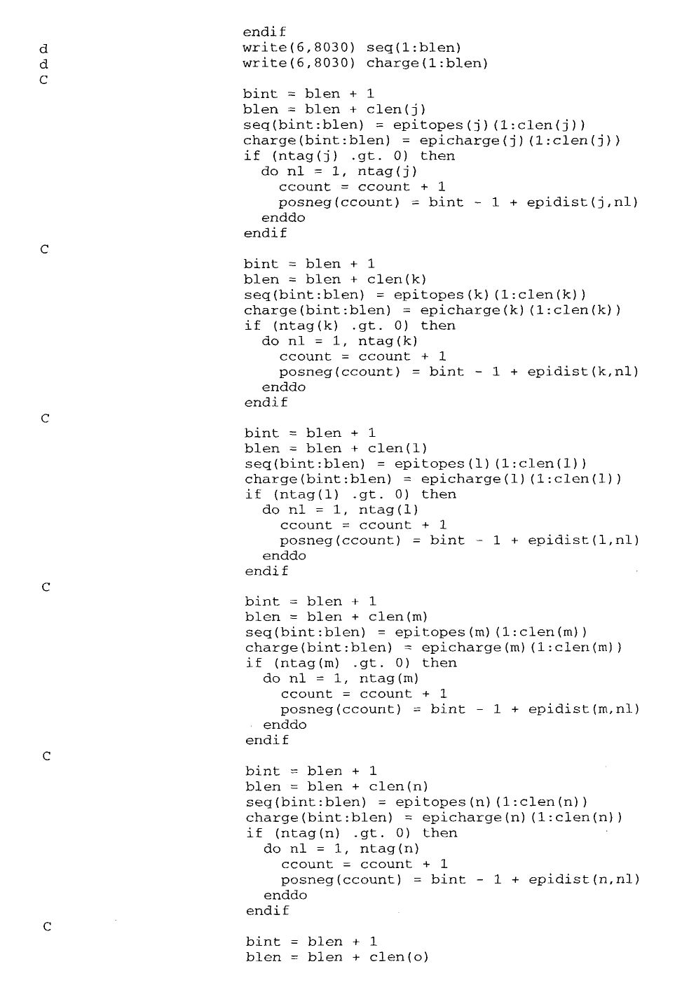

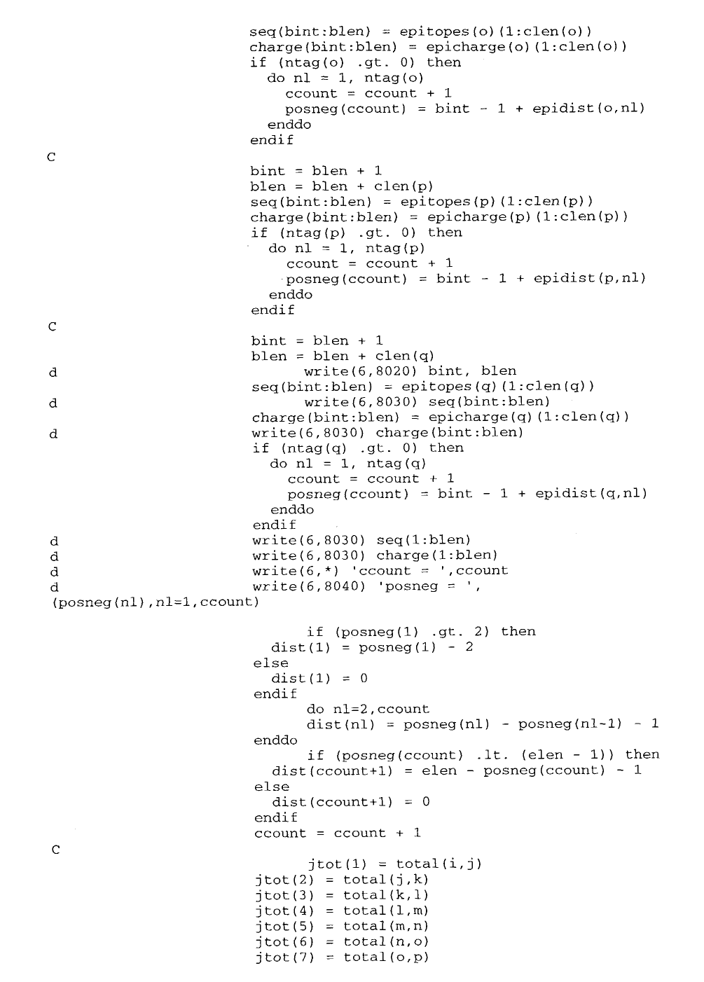

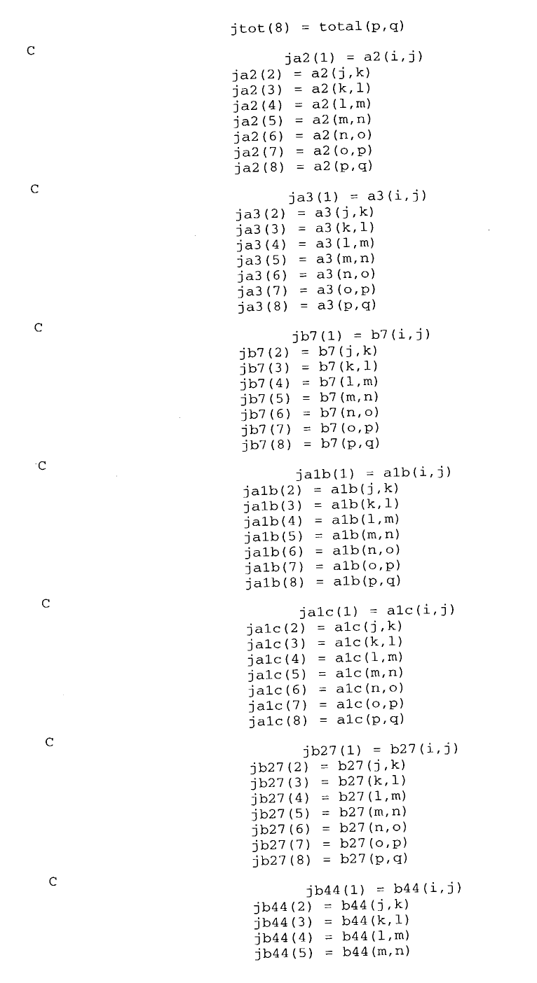

Examples of such algorithms are shown in FIGS. 1-2. FIG. 1 shows a program to calculate allowed linker (or spacer) length and identities of residues allowed at each position. This program is in the computer language Fortran source, but it is understood that other computer languages may be used. A sample of required data has been hard-coded into this program. The output from this program is listed in Table 8.

TABLE 8

| | | | | | | | | | | | | | | | | | | | | | | | |
|---|---|---|---|---|---|---|---|---|---|---|---|---|---|---|---|---|---|---|---|---|---|---|---|
| 1 - | 2 | 4: | 1 ₌ | G | A | P | C | M | V | I | F | Y | W | H | s | T | N | D | Q | E | K | R | |
| 1 - | 2 | 4: | 2 = | G | A | P | C | M | V | I | L | F | Y | W | H | S | T | N | D | Q | E | K | R |
| 1 - | 2 | 4: | 3 = | G | A | P | C | M | I | F | Y | W | H | s | T | N | D | Q | E | K | R | | |
| 1 - | 2 | 4: | 4 = | G | A | P | C | M | I | F | Y | W | H | s | T | N | D | Q | E | K | R | | |

FIG. 2 shows a program to find all possible epitope orders once spacer possibilities are determined for the epitopes in Table 5 using the information in Example 82.

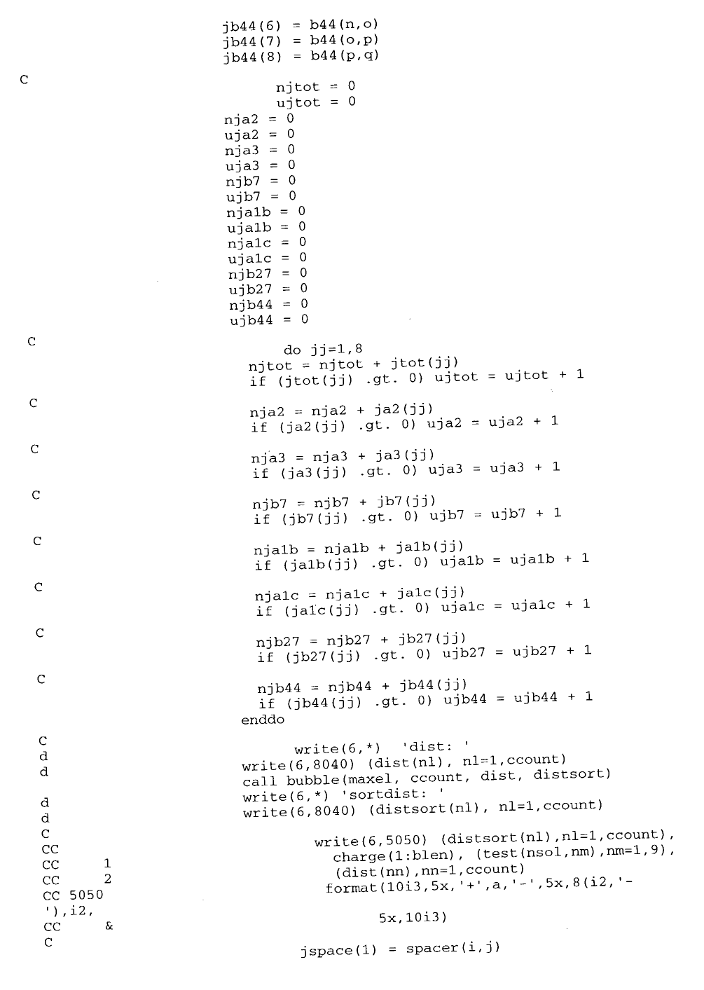

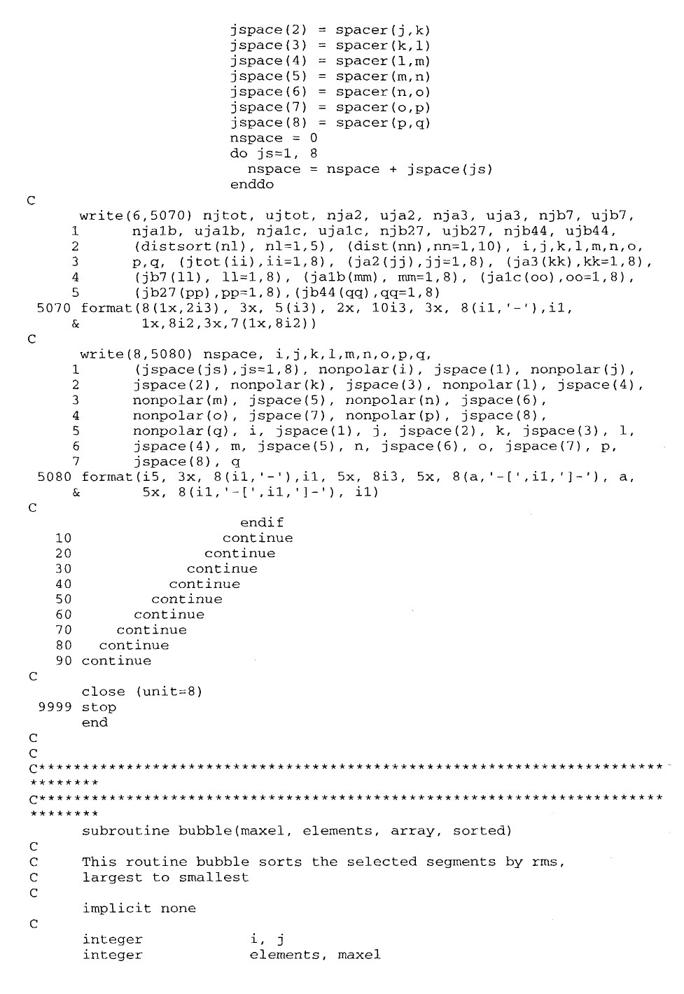

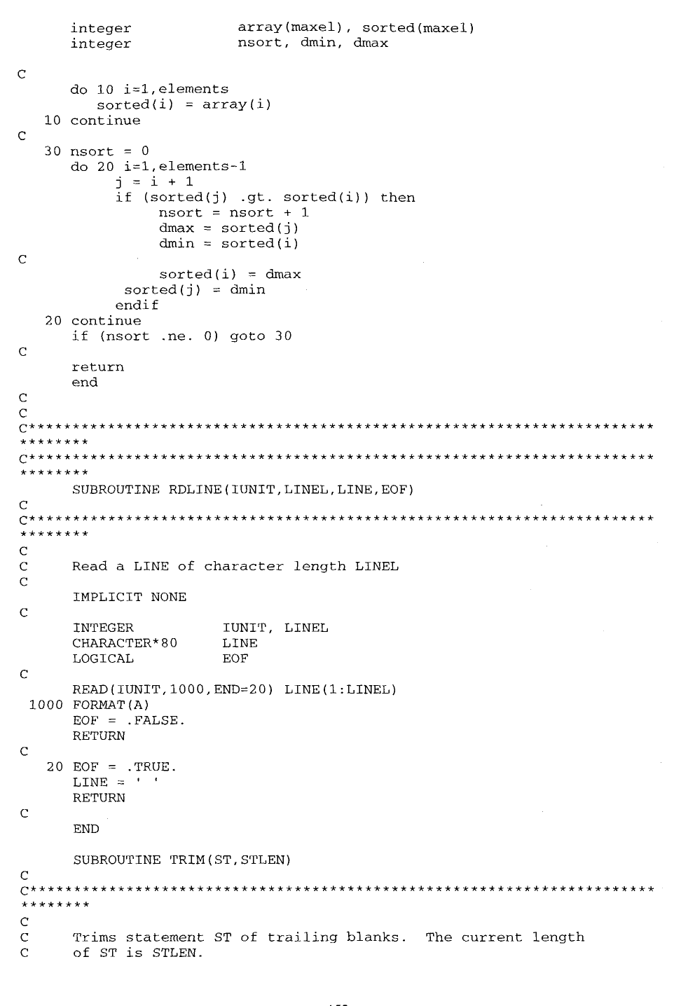

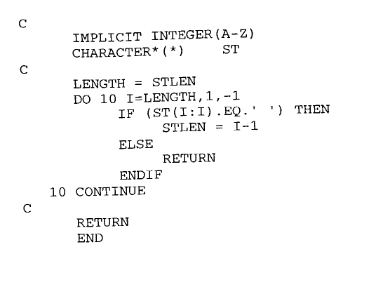

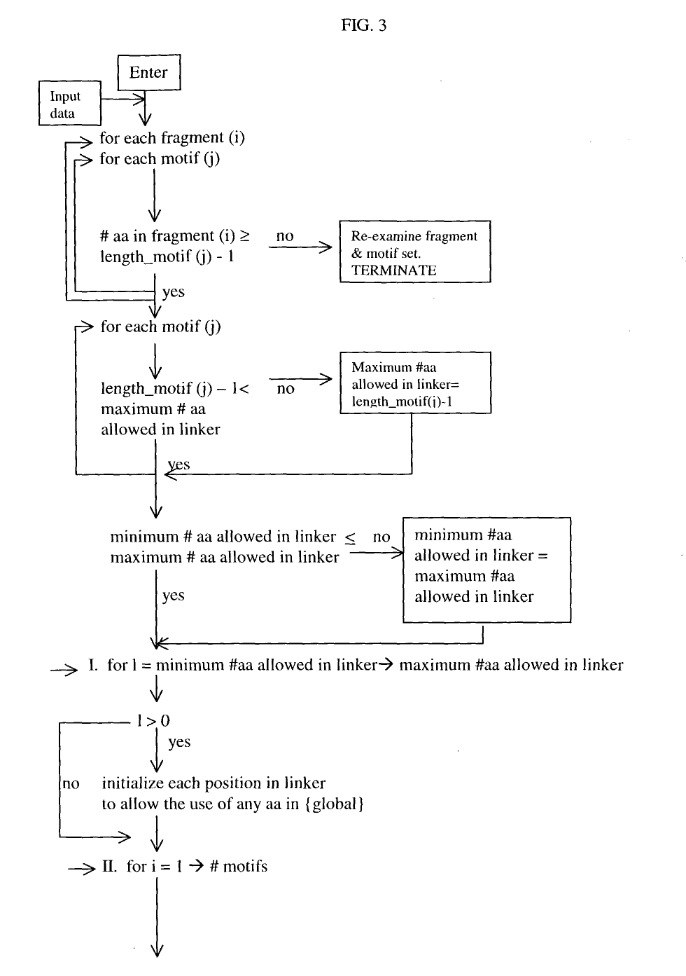

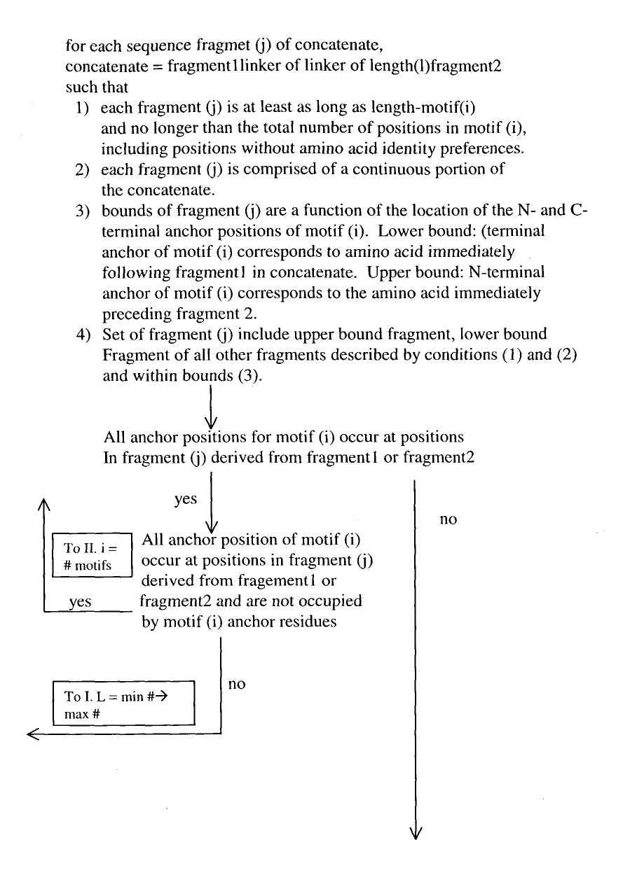

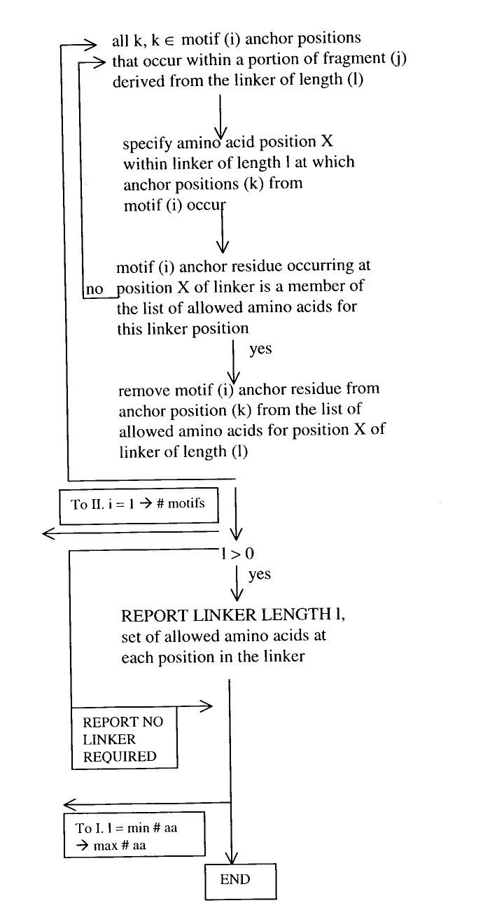

FIG. 3 is a flowchart, to be used as a program, subroutine or function to calculate the linker length and linker composition which avoids the creation of junction epitopes betweentwo amino acid fragments, fragment 1 & fragment 2. Epitopes are created when motifs are satisfied.

The possible uses of this flowchart include:

(1) calculate linker requirements between fragments, fragment 1 & fragment 2, derived from noncontiguous sequence

(2) calculate amino acid insertion requires at any point in a contiguous sequence where sequence to the left of the arbitrary insertion point is treated as fragment 1 and the sequence to the right is treated as fragment 2

(3) calculate sequence modifications which will eliminate junction epitopes after a contigous segment of amino acids has been deleted, where sequence to the left of the deletion is treated as fragment1 & sequence to the right of the deletion is treated as fragment 2

Abbreviations included in the flowchart are as follows:

&=and

aa=amino acid;

#=number

length_motifs)=#aa between & including N-terminal & C-terminal anchors of motif(j)

{global}=all possible amino acid identities permitted in linker

anchor position=the location of a preferred amino acid within a motif

anchor residue=the identity of the amino acid at an anchor position

concatenate=the conceptual entity created when fragment 2 is appended to the linker and the linker is appended to fragment 1.

INPUT DATA for the flowchart includes

fragments: fragment 1 sequence, fragment 2 sequence, number of amino acids in each fragment.

motifs: number of anchors per motif, position of anchor in each motif, amino acid preferred at each anchor position in each motif.

linker: max. number of amino acids in linker, global set of amino acid types which can be incorporated at each position in the linker. min. number of amino acids in linker≧0.

### EXAMPLES

In the examples listed below, the number of junctional epitopes are calculated for each possible pairing of the epitopes listed in Table 5 that might be included in a polyepitope, using linkers of zero to six amino acids between epitopes. If there is found to be one or more lengths of linkers which would result in zero junctional epitopes (ie., junctional epitopes being epitopes which span the epitopes of the pair and having a N-terminal anchor residue in the N-terminal epitope, and a C-terminal anchor in the C-terminal epitope) for any given pairing, then linkers of said length must not be used if the creation of junctional epitopes is to be controlled in a. Otherwise, a lindker of said length is allowed.

The amino acids to be avoided at each position in the linker are determined in consideration of the anchor residues for motifs corresponding to the MHC subtypes of interest occurring in the N-terminal vaccine epitopes C-terminal for motifs corresponding to the MHC subtypes of interest occurring in the C-terminal vaccine epitopes. As described above, one counts from potential N-terminal anchors in the N-terminal vaccine epitope to positions in the linker where the corresponding C-terminal anchor(s) must lie for each motif applied. At the so-identified positions in the linker, residues that could function as C-terminal anchors for the potential N-terminal anchors must not be used at that position in the linker to avoid undesired epitopes with N-terminal anchors in the N-terminal epitope and with C-terminal anchors in the linker. Similarly, one counts from potential C-terminal anchors in the C-terminal vaccine epitope to positions in the linker where the corresponding N-terminal anchor(s) must lie for each motif applied. At the so-identified positions in the linker, residues that could function as N-terminal anchors corresponding to the potential C-terminal anchors must not be used in the linker to avoid undesired epitopes with C-terminal anchors in the C-terminal epitope and with N-terminal anchors in the linker.

Lists of prohibited amino acid residues are compiled for each position in the linker, and for each position in the linker, lists of residues prohibited as they might constitute N-terminal anchors and residues that might constitute C-terminal anchors are merged. In the examples below, the merged lists are shown beneath a dashed line in which each dash represents a position in the linker. Prohibited amino acid residues are shown position by position in the linker, with the vertical column beneath the leftmost dash representing those amino acids that must be avoided at the N-terminal position of the linker, and so on to the rightmost dash, under which is listed those amino acids that must be avoided at the C-terminal position of the linker. When no amino acids are listed under a dash, it indicates that there are no constraints on which amino acid can be inserted at the position corresponding to that dash in the linker without producing an undesired epitope. Any amino acid other than those listed beneath the position can be introduced at that position in the linker without de novo generation of an epitope.

The nominclature for factors of the HLA system has been updated. Current nominclature for the HLA system can be found in Marsh et al., Nomenclature for Factors of the HLA System, 2002. Human Immunology 63: 1213-1268 (2002). The data presented in examples 1-81 will be used to design several example polyepitopes that link all of the epitopes of Table 5 together such that the polyepitopes contain no epitopes in addition to the vaccine epitopes of Table 5. The design process will consist of selecting epitopes to abut each other with linkers between them selected from data generated in Examples 1-81 such that the resulting polyepitope contains no epitopes in addition to the vaccine epitopes of Table 5 that were produced as the result of abutting the epitopes of Table 5 to one another, or as the result of specific linker amino acid content. In this embodiment, none of the epitopes of Table 5 will be used more than once in the polyepitope. However, it is possible to incorporate multiple copies of epitopes. In this embodiment, the shortest linkers that can be used to produce junctures with no undesired epitopes will be preferentially chosen.

Where the data of examples 1-81 indicate that several linkers of differing amino acid lengths might be used to eliminate junctional epitopes between two vaccine epitopes, the shortest linker will be chosen in this design example. Any linker length less than or equal to one plus the number of amino acids between the N-terminal anchor position and the C-terminal anchor position of the motif applied which is defined with the greatest number of amino acids between its N— and C-terminal anchor positions might be considered. If there is found to be one or more lengths of linkers which could result in the creation of zero junctional epitopes (i.e., junctional epitopes being epitopes which span the epitopes of the pair and having a N-terminal anchor residue in the N-terminal (positionally first of the pair) epitope, and a C-terminal anchor in the C-terminal (positionally last of the pair) [to p5 1] p51 epitope) for any given epitope pairing, then linkers of said length could be used between the epitope pair if the creation of junctional epitopes is to be controlled in a previously specified manner (eg the creation of A2, A3 & B7 junctional epitopes is to be avoided). Such linker lengths are termed allowed for a given epitope pair. Otherwise, a linker of said length is termed disallowed.

In another embodiment, the linker itself is as long or longer as the longest motif, such that any motif that begins in the first epitope cannot end in the second epitope.

To aid in the design exercise, all of the epitope pairs that can be abutted to one another with a linker of zero to six amino acids between them such that there are no junctional epitopes spanning the linker or with their N— or C-terminal anchor residues in the linker are listed in Example 82. Example 82 also lists the minimal linker length in amino acids that can be inserted between the two epitopes and result in no junctional epitopes.

In Examples 83-85 three polyepitope configurations that meet the design parameters of this exercise (representing each vaccine epitope of Table 5 once, having no epitopes other than the vaccine epitopes of Table 5 present, and for any given epitope pairing, using the shortest linker that will eliminate junctional epitopes with a N-terminal anchor in the N-terminal vaccine epitope or a C-terminal anchor in the C-terminal vaccine epitope). Polyepitopes are assembled using the epitope pairings and linker lengths specified by Example 82. While we provide only three example configurations, it will be apparent to persons skilled in the art that many polyepitope configurations that satisfy the parameters of the design exercise could be compiled from the data of Example 82. The number of possible satisfactory unique orders in which the epitope is linked, enabled by the data of Examples 1-82, is 14,592, including junctional epitopes.

In Examples 86-90, 5 polyepitopes conforming to the configuration set forth in Example 83 are shown. The vaccine epitopes are shown as they were in Example 83, and the specific amino acids for each position in the linkers are shown, using the single letter code for amino acids, between the vaccine epitopes. As described above, each of these specific polyepitopes exhibit no juctional epitopes with the N-terminal anchor in an N-terminal vaccine epitope and a C-terminal anchor in the following vaccine epitope. Additionally, the specific amino acids in the linkers were selected so that they will generate de novo no undesired epitopes that have N-terminal or C-terminal anchors in the linker. These amino acids are naturally occuring amino acids that do not appear as prohibited amino acids in linkers for the particular vaccine epitope pairings shown in examples 1-81. It will be apparent to one skilled in the art that while we show 5 examples of polyepitopes matching the configuragion in example 83, the method described herein using the information generated in examples 1-81, will allow identification of a vast number of polyepitopes (4,668,560,953,056,000 individual polyepitopes) containing only the vaccine epitopes of Table 5 and satisfying the configuration of Example 83.

Example 84 describes yet another configuration predicted out of the information of Examples 1-82 to contain no epitopes other than the vaccine epitopes of Table 5. The data generated in Examples 1-82 predict a vast number of polyepitopes matching the configuration of Example 84, and containing only the vaccine epitopes of Table 5 (540,441,508,422,816,000 individual polyepitopes). Five examples of such polyepitopes are shown in Examples 91-95.

Example 85 describes yet another configuration predicted out of the information of Examples 1-82 to contain no epitopes other than the vaccine epitopes of Table 5. The data generated in Examples 1-82 predict a vast number of polyepitopes matching the configuration of Example 85, and containing only the vaccine epitopes of Table 5 (135,536,796,720 individual polyepitopes). Five examples of such polyepitopes are shown in Examples 96-100.

### Example 1

Junctional epitopes between epitopes 1 and 1 as a function of linker length. Epitope 1 at N-end, Epitope 1 at C-end

| | | | | | | | | | | | | | | | | | | | | | | | |
|---|---|---|---|---|---|---|---|---|---|---|---|---|---|---|---|---|---|---|---|---|---|---|---|
| K | L | C | P | V | Q | L | W | V | _{—} | _{—} | _{—} | _{—} | _{—} | _{—} | K | L | C | P | V | Q | L | W | V |
| | A₂ | | B₇ | A₃ | | A₂ | | A₃ | | | | | | | A₃ | A₂ | | | A₂ | | A₂ | | A₂ |
| | A₃ | | | | | A₃ | | | | | | | | | B₂₇ | B₇ | | | B₇ | | B₇ | | B₇ |
| | | | | | | | | | | | | | | | | B₂₇ | | | | | B₂₇ | | |

Linker Length in Amino Acids

| Class I MHC | 0 | 1 | 2 | 3 | 4 | 5 | 6 |
|---|---|---|---|---|---|---|---|
| A₂ | 1 | 1 | 0 | 1 | 1 | 0 | 0 |
| A₃ | 1 | 0 | 1 | 1 | 1 | 1 | 1 |
| B₇ | 1 | 1 | 0 | 0 | 0 | 0 | 0 |
| Total | 3 | 2 | 1 | 2 | 2 | 1 | 1 |

Allowing linkers of 0 to 6 amino acids between epitopes, the 1(N-terminal)-1(C-terminal) epitope pairing cannot be used in a polyepitope with zero junctional epitopes for the Class I haplotype motifs considered here.

### Example 2

Junctional epitopes between epitopes 1 and 2 as a function of linker length. Epitope 1 at N-end, Epitope 2 at C-end

| | | | | | | | | | | | | | | | | | | | | | | | | | |
|---|---|---|---|---|---|---|---|---|---|---|---|---|---|---|---|---|---|---|---|---|---|---|---|---|---|
| K | L | C | P | V | Q | L | W | V | _{—} | _{—} | _{—} | _{—} | _{—} | _{—} | A | K | F | V | A | A | W | T | L | K | AAA |
| | A₂ | | B₇ | A₃ | | A₂ | | A₃ | | | | | | | | A₃ | B₇ | B₇ | | | | | | | |
| | A₃ | | | | | A₃ | | | | | | | | | | B₂₇ | A₂₄ | A₂ | | | | | | | |

B₂₇
B₄₄

Linker Length in Amino Acids

| Class I MHC | 0 | 1 | 2 | 3 | 4 | 5 | 6 |
|---|---|---|---|---|---|---|---|
| A₂ | 0 | 1 | 1 | 0 | 0 | 0 | 0 |
| A₃ | 0 | 1 | 1 | 1 | 1 | 1 | 1 |
| B₇ | 1 | 0 | 0 | 0 | 0 | 0 | 0 |
| Total | 1 | 2 | 2 | 1 | 1 | 1 | 1 |

Allowing linkers of 0 to 6 amino acids between epitopes, the 1(N-terminal)-2(C-terminal) epitope pairing cannot be used in a polyepitope with zero junctional epitopes for the Class I haplotype motifs considered here.

### Example 3

Junctional epitopes between epitopes 1 and 3 as a function of linker length. Epitope 1 at N-end, Epitope 3 at C-end

| | | | | | | | | | | | | | | | | | | | | | | | |
|---|---|---|---|---|---|---|---|---|---|---|---|---|---|---|---|---|---|---|---|---|---|---|---|
| K | L | C | P | V | Q | L | W | V | _{—} | _{—} | _{—} | _{—} | _{—} | _{—} | K | V | A | E | L | V | H | F | L |
| | A₂ | | B₇ | A₃ | | A₂ | | A₃ | | | | | | | A₃ | A₂ | | | A₂ | A₂ | | B₇ | |
| | A₃ | | | | | A₃ | | | | | | | | | B₂₇ | B₇ | | | B₇ | B₇ | | A₂₄ | |

| | |
|---|---|
| B₂₇ | B₂₇ |
| | B₄₄ |

Linker Length in Amino Acids

| Class I MHC | 0 | 1 | 2 | 3 | 4 | 5 | 6 |
|---|---|---|---|---|---|---|---|
| A₂ | 2 | 1 | 0 | 1 | 1 | 0 | 0 |
| A₃ | 1 | 0 | 1 | 1 | 1 | 1 | 1 |
| B₇ | 1 | 1 | 0 | 0 | 0 | 0 | 0 |
| Total | 4 | 2 | 1 | 2 | 2 | 1 | 1 |

Allowing linkers of 0 to 6 amino acids between epitopes, the 1(N-terminal)-3 (C-terminal) epitope pairing cannot be used in a polyepitope with zero junctional epitopes for the Class I haplotype motifs considered here.

### Example 4

Junctional epitopes between epitopes 1 and 4 as a function of linker length. Epitope 1 at N-end, Epitope 4 at C-end

| | | | | | | | | | | | | | | | | | | | | | | | |
|---|---|---|---|---|---|---|---|---|---|---|---|---|---|---|---|---|---|---|---|---|---|---|---|
| K | L | C | P | V | Q | L | W | V | _{—} | _{—} | _{—} | _{—} | _{—} | _{—} | V | V | L | G | V | V | F | G | I |
| | A₂ | | B₇ | A₃ | | A₂ | | A₃ | | | | | | | A₂ | A₂ | A₂ | | A₂ | A₂ | A₂₄ | | |
| | A₃ | | | | | A₃ | | | | | | | | | B₇ | B₇ | B₇ | | B₇ | B₇ | B₂₇ | | |
| | | | | | | | | | | | | | | | | | B₂₇ | | | | B₄₄ | | |
| | | | | | | | | | | | | | | | | | | | | | B₇ | | |

B₇

Linker Length in Amino Acids

| Class I MHC | 0 | 1 | 2 | 3 | 4 | 5 | 6 |
|---|---|---|---|---|---|---|---|
| A₂ | 3 | 1 | 1 | 2 | 2 | 1 | 0 |
| A₃ | 0 | 0 | 0 | 0 | 0 | 0 | 0 |
| B₇ | 2 | 2 | 1 | 0 | 0 | 0 | 0 |
| Total | 5 | 3 | 2 | 2 | 2 | 1 | 0 |

The 1(N-terminal)-4(C-terminal) epitope pairing can be used in a polyepitope with zero junctional epitopes for the Class I haplotype motifs considered here. Linkers of six amino acids in length will produce no junctional epitopes.

To avoid junctional epitopes, the following amino acids MUST be avoided in a six amino acid linker between epitopes 1(N-terminal)-4(C-terminal). The linker is shown from left to right, with the most N-terminal residue of the linker occuring at the left, and the most C-terminal residue occuring to the right. Amino acids to be avoided are shown position by position in the linker.

| | | | | | | | |
|---|---|---|---|---|---|---|---|
| N-End | _{—} | _{—} | _{—} | _{—} | _{—} | _{—} | C-End |
| | V | L | L | K | V | V | |
| | I | I | I | R | I | I | |
| | M | M | M | L | L | L | |
| | K | V | V | M | K | R | |
| | R | F | F | P | M | P | |
| | L | P | K | | P | E | |
| | P | R | R | | R | K | |
| | | | P | | Y | Y | |
| | | | | | E | | |

### Example 5

Junctional epitopes between epitopes 1 and 5 as a function of linker length. Epitope 1 at N end, Epitope 5 at C end

| | | | | | | | | | | | | | | | | | | | | | | | | |
|---|---|---|---|---|---|---|---|---|---|---|---|---|---|---|---|---|---|---|---|---|---|---|---|---|
| K | L | C | P | V | Q | L | W | V | _{—} | _{—} | _{—} | _{—} | _{—} | _{—} | Y | L | Q | L | V | F | G | I | E | V |
| | A₂ | | B₇ | A₃ | | A₂ | | A₃ | | | | | | | A_{1b} | A₂ | | A₂ | A₂ | B₇ | | A₂ | | |
| | A₃ | | | | | A₃ | | | | | | | | | A_{1c} | B₇ | | B₇ | B₇ | B₂₇ | | B₇ | | |
| | | | | | | | | | | | | | | | B₄₄ | B₂₇ | | B₂₇ | | A₂₄ | | | | |
| | | | | | | | | | | | | | | | | | | | | B₄₄ | | | | |

Linker Length in Amino Acids

| Class I MHC | 0 | 1 | 2 | 3 | 4 | 5 | 6 |
|---|---|---|---|---|---|---|---|
| A₂ | 1 | 2 | 1 | 1 | 1 | 0 | 0 |
| A₃ | 0 | 0 | 0 | 0 | 0 | 0 | 0 |
| B₇ | 1 | 1 | 0 | 0 | 0 | 0 | 0 |
| Total | 2 | 3 | 1 | 1 | 1 | 0 | 0 |

The 1(N-terminal)-5(C-terminal) epitope pairing can be used in a polyepitope with zero junctional epitopes for the Class I haplotype motifs considered here. Linkers of five and six amino acids in length will produce no junctional epitopes.

To avoid junctional epitopes, the following amino acids MUST be avoided in a five or six amino acid linker between epitopes 1(N-terminal)-5(C-terminal). The linker is shown from left to right, with the most N-terminal residue of the linker occuring at the left, and the most C-terminal residue occuring to the right. Amino acids to be avoided are shown position by position in the linker.

| | | | | | | |
|---|---|---|---|---|---|---|
| N-End | _{—} | _{—} | _{—} | _{—} | _{—} | C-End |
| | V | L | L | K | V | |
| | I | I | Y | R | I | |
| | L | M | E | Y | K | |
| | K | V | I | E | R | |
| | R | F | M | P | L | |
| | M | P | V | | P | |
| | P | R | F | | M | |
| | | | K | | | |
| | | | R | | | |
| | | | P | | | |

### Example 6

Junctional epitopes between epitopes 1 and 6 as a function of linker length. Epitope 1 at N end, Epitope 6 at C end

| | | | | | | | | | | | | | | | | | | | | | | | |
|---|---|---|---|---|---|---|---|---|---|---|---|---|---|---|---|---|---|---|---|---|---|---|---|
| K | L | C | P | V | Q | L | W | V | _{—} | _{—} | _{—} | _{—} | _{—} | _{—} | I | M | I | G | V | L | V | G | V |
| | A₂ | | B₇ | A₃ | | A₂ | | A₃ | | | | | | | A₂ | B₇ | A₂ | | A₂ | A₂ | A₂ | | A₂ |
| | A₃ | | | | | A₃ | | | | | | | | | B₇ | | B₇ | | B₇ | B₇ | B₇ | | |
| | | | | | | | | | | | | | | | | | | | | B₂₇ | | | |

Linker Length in Amino Acids

| Class I MHC | 0 | 1 | 2 | 3 | 4 | 5 | 6 |
|---|---|---|---|---|---|---|---|
| A₂ | 3 | 1 | 1 | 1 | 1 | 1 | 0 |
| A₃ | 0 | 0 | 0 | 0 | 0 | 0 | 0 |
| B₇ | 2 | 2 | 1 | 0 | 0 | 0 | 0 |
| Total | 5 | 3 | 2 | 1 | 1 | 1 | 0 |

The 1(N-terminal)-6(C-terminal) epitope pairing can be used in a polyepitope with zero junctional epitopes for the Class I haplotype motifs considered here. Linkers of six amino acids in length will produce no junctional epitopes.

To avoid junctional epitopes, the following amino acids MUST be avoided in a six amino acid linker between epitopes 1(N-terminal)-6(C-terminal). The linker is shown from left to right, with the most N-terminal residue of the linker occuring at the left, and the most C-terminal residue occuring to the right. Amino acids to be avoided are shown position by position in the linker.

| | | | | | | | |
|---|---|---|---|---|---|---|---|
| N-End | _{—} | _{—} | _{—} | _{—} | _{—} | _{—} | C-End |
| | V | L | L | K | V | V | |
| | I | I | I | R | I | I | |
| | L | M | M | L | L | L | |
| | K | V | V | M | K | K | |
| | R | F | F | P | R | R | |
| | M | P | K | | M | M | |
| | P | R | R | | P | P | |
| | | K | P | | | | |

### Example 7

Junctional epitopes between epitopes 1 and 7 as a function of linker length. Epitope 1 at N end, Epitope 7 at C end

| | | | | | | | | | | | | | | | | | | | | | | | |
|---|---|---|---|---|---|---|---|---|---|---|---|---|---|---|---|---|---|---|---|---|---|---|---|
| K | L | C | P | V | Q | L | W | V | _{—} | _{—} | _{—} | _{—} | _{—} | _{—} | Y | L | S | G | A | N | L | N | V |
| | A₂ | | B₇ | A₃ | | A₂ | | A₃ | | | | | | | A_{1b} | A₂ | | | | | A₂ | | |
| | A₃ | | | | | A₃ | | | | | | | | | A_{1c} | B₇ | | | | | B₇ | | |
| | | | | | | | | | | | | | | | B₄₄ | B₂₇ | | | | | B₂₇ | | |

Linker Length in Amino Acids

| Class I MHC | 0 | 1 | 2 | 3 | 4 | 5 | 6 |
|---|---|---|---|---|---|---|---|
| A₂ | 0 | 0 | 0 | 1 | 1 | 0 | 0 |
| A₃ | 0 | 0 | 0 | 0 | 0 | 0 | 0 |
| B₇ | 1 | 1 | 0 | 0 | 0 | 0 | 0 |
| Total | 1 | 1 | 0 | 1 | 1 | 0 | 0 |

The 1(N-terminal)-7(C-terminal) epitope pairing can be used in a polyepitope with zero junctional epitopes for the Class I haplotype motifs considered here. Linkers of two, five, or six amino acids in length will produce no junctional epitopes.

To avoid junctional epitopes, the following amino acids MUST be avoided in a two amino acid linker between epitopes 1(N-terminal)-7(C-terminal). The linker is shown from left to right, with the most N-terminal residue of the linker occuring at the left, and the most C-terminal residue occuring to the right. Amino acids to be avoided are shown position by position in the linker.

| | | | |
|---|---|---|---|
| N-End | _{—} | _ | C-End |
| | V | L | |
| | I | I | |
| | L | M | |
| | K | V | |
| | R | F | |
| | P | P | |
| | M | R | |

### Example 8

Junctional epitopes between epitopes 1 and 8 as a function of linker length. Epitope pair 8: Epitope 1 at N end, Epitope 8 at C end

| | | | | | | | | | | | | | | | | | | | | | | | |
|---|---|---|---|---|---|---|---|---|---|---|---|---|---|---|---|---|---|---|---|---|---|---|---|
| K | L | C | P | V | Q | L | W | V | _{—} | _{—} | _{—} | _{—} | _{—} | _{—} | R | L | L | Q | E | T | E | L | V |
| | A₂ | | B₇ | A₃ | | A₂ | | A₃ | | | | | | | A₃ | A₂ | A₂ | | | | | A₂ | |
| | A₃ | | | | | A₃ | | | | | | | | | B₂₇ | B₇ | B₇ | | | | | B₇ | |
| | | | | | | | | | | | | | | | | B₂₇ | B₂₇ | | | | | B₂₇ | |

Linker Length in Amino Acids

| Class I MHC | 0 | 1 | 2 | 3 | 4 | 5 | 6 |
|---|---|---|---|---|---|---|---|
| A₂ | 0 | 0 | 1 | 2 | 1 | 0 | 0 |
| A₃ | 1 | 0 | 1 | 1 | 1 | 1 | 1 |
| B₇ | 2 | 1 | 0 | 0 | 0 | 0 | 0 |
| Total | 3 | 1 | 2 | 3 | 2 | 1 | 1 |

Allowing linkers of 0 to 6 amino acids between epitopes, the 1(N-terminal)-8 (C-terminal) epitope pairing cannot be used in a polyepitope with zero junctional epitopes for the Class I haplotype motifs considered here.

### Example 9

Junctional epitopes between epitopes 1 and 9 as a function of linker length. Epitope 1 at N end, Epitope 9 at C end

| | | | | | | | | | | | | | | | | | | | | | | | |
|---|---|---|---|---|---|---|---|---|---|---|---|---|---|---|---|---|---|---|---|---|---|---|---|
| K | L | C | P | V | Q | L | W | V | _{—} | _{—} | _{—} | _{—} | _{—} | _{—} | S | M | P | P | P | G | T | R | V |
| | A₂ | | B₇ | A₃ | | A₂ | | A₃ | | | | | | | | B₇ | | | | | | A₃ | |
| | A₃ | | | | | A₃ | | | | | | | | | | | | | | | | B₂₇ | |

Linker Length in Amino Acids

| Class I MHC | 0 | 1 | 2 | 3 | 4 | 5 | 6 |
|---|---|---|---|---|---|---|---|
| A₂ | 0 | 0 | 0 | 0 | 0 | 0 | 0 |
| A₃ | 1 | 0 | 0 | 0 | 0 | 0 | 0 |
| B₇ | 1 | 1 | 0 | 0 | 0 | 0 | 0 |
| Total | 1 | 1 | 0 | 0 | 0 | 0 | 0 |

The 1(N-terminal)-9(C-terminal) epitope pairing can be used in a polyepitope with zero junctional epitopes for the Class I haplotype motifs considered here. Linkers of two, three, fourt, five or six amino acids in length will produce no junctional epitopes.

To avoid junctional epitopes, the following amino acids MUST be avoided in a two amino acid linker between epitopes 1(N-terminal)-9(C-terminal). The linker is shown from left to right, with the most N-terminal residue of the linker occuring at the left, and the most C-terminal residue occuring to the right. Amino acids to be avoided are shown position by position in the linker.

```
N-End_{— —}C-End
          V L
          I I
          L M
          K V
          R F
              T
              R
```

### Example 10

Junctional epitopes between epitopes 2 and 1 as a function of linker length. Epitope 2 at N end, Epitope 1 at C end

| | | | | | | | | | | | | | | | | | | | | | | | | |
|---|---|---|---|---|---|---|---|---|---|---|---|---|---|---|---|---|---|---|---|---|---|---|---|---|
| AKFV | A | A | W | T | L | K | A | A | A | _{—} | _{—} | _{—} | _{—} | _{—} | _{—} | K | L | C | P | V | Q | L | W | V |
| | | | | A_{1b} | A₂ | | | | | | | | | | | A₃ | A₂ | | | A₂ | | A₂ | | |
| | | | | A₃ | A₃ | | | | | | | | | | | B₂₇ | B₇ | | | B₇ | | B₇ | | |
| | | | | | | | | | | | | | | | | | B₂₇ | | | | | B₂₇ | | |

Linker Length in Amino Acids

| Class I MHC | 0 | 1 | 2 | 3 | 4 | 5 | 6 |
|---|---|---|---|---|---|---|---|
| A₂ | 0 | 1 | 1 | 0 | 0 | 0 | 0 |
| A₃ | 0 | 1 | 2 | 1 | 0 | 0 | 0 |
| A_{1b} | 0 | 0 | 0 | 0 | 0 | 0 | 0 |
| Total | 0 | 2 | 3 | 1 | 0 | 0 | 0 |

The 2(N-terminal)-1(C-terminal) epitope pairing can be used in a polyepitope with zero junctional epitopes for the Class I haplotype motifs considered here. Linkers of zero, four, five, or six amino acids in length will produce no junctional epitopes. As there are no amino acids in a zero amino acid linker, there are no constraints on the amino acids used for a zero length linker.

### Example 11

Junctional epitopes between epitopes 2 and 2 as a function of linker length. Epitope 2 at N end, Epitope 2 at C end

| | | | | | | | | | | | | | | | | | | | | | | | | |
|---|---|---|---|---|---|---|---|---|---|---|---|---|---|---|---|---|---|---|---|---|---|---|---|---|
| AKFVA | AW | T | L | K | A | A | A | _{—} | _{—} | _{—} | _{—} | _{—} | _{—} | A | K | F | V | A | A | W | T | L | KAA | A |
| | | A₃ | A₂ | | | | | | | | | | | | A₃ | B₂₇ | A₂ | | | | | | | |
| | | A_{1b} | A₃ | | | | | | | | | | | | B₇ | B₇ | B₇ | | | | | | | |

A₂₄
B₄₄

Linker Length in Amino Acids

| Class I MHC | 0 | 1 | 2 | 3 | 4 | 5 | 6 |
|---|---|---|---|---|---|---|---|
| A₂ | 1 | 0 | 0 | 0 | 0 | 0 | 0 |
| A₃ | 1 | 2 | 1 | 0 | 0 | 0 | 0 |
| A_{1b} | 0 | 0 | 0 | 0 | 0 | 0 | 0 |
| Total | 2 | 2 | 1 | 0 | 0 | 0 | 0 |

The 2(N-terminal)-2(C-terminal) epitope pairing can be used in a polyepitope with three, four, five, or six junctional epitopes for the Class I haplotype motifs considered here.

### Example 11

### Extension

To avoid junctional epitopes, the following amino acids MUST be avoided in a three amino acid linker between epitopes 2(N-terminal)-2(C-terminal). The linker is shown from left to right, with the most N-terminal residue of the linker occurring at the left, and the most C-terminal residue occurring to the right. Amino acids to be avoided are shown position by position in the linker

| | | | | |
|---|---|---|---|---|
| N-End | _{—} | _{—} | _{—} | C-End |
| | | K | V | |
| | | R | I | |
| | | Y | L | |
| | | | K | |
| | | | R | |
| | | | Y | |

### Example 12

Junctional epitopes between epitopes 2 and 3 as a function of linker length. Epitope pair 12: Epitope 2 at N end, Epitope 3 at C-end

| | | | | | | | | | | | | | | | | | | | | | | | |
|---|---|---|---|---|---|---|---|---|---|---|---|---|---|---|---|---|---|---|---|---|---|---|---|
| AKFVA | A | W | T | L | K | A | A | A | _{—} | _{—} | _{—} | _{—} | _{—} | _{—} | K | V | A | E | L | V | H | F | L |
| | | | A₃ | A₂ | | | | | | | | | | | A₃ | A₂ | | | A₂ | A₂ | | B₇ | |
| | | | A_{1b} | A₃ | | | | | | | | | | | B₂₇ | B₇ | | | B₇ | B₇ | | B₂₇ | |

| | |
|---|---|
| B₂₇ | A₂₄ |
| | B₄₄ |

Linker Length in Amino Acids

| Class I MHC | 0 | 1 | 2 | 3 | 4 | 5 | 6 |
|---|---|---|---|---|---|---|---|
| A₂ | 0 | 1 | 1 | 0 | 0 | 0 | 0 |
| A₃ | 0 | 1 | 2 | 1 | 0 | 0 | 0 |
| A_{1b} | 0 | 0 | 0 | 0 | 0 | 0 | 0 |
| Total | 0 | 2 | 3 | 1 | 0 | 0 | 0 |

The 2(N-terminal)-3(C-terminal) epitope pairing can be used in a polyepitope with zero junctional epitopes for the Class I haplotype motifs considered here. Linkers of zero, four, five, or six amino acids in length will produce no junctional epitopes. As there are no amino acids in a zero amino acid linker, there are no constraints on the amino acids used in a zero length linker.

### Example 13

Junctional epitopes between epitopes 2 and 4 as a function of linker length. Epitope 2 at N-end, Epitope 4 at C-end

| | | | | | | | | | | | | | | | | | | | | | | | |
|---|---|---|---|---|---|---|---|---|---|---|---|---|---|---|---|---|---|---|---|---|---|---|---|
| AKFVA | A | W | T | L | K | A | A | A | _{—} | _{—} | _{—} | _{—} | _{—} | _{—} | V | V | L | G | V | V | F | G | I |
| | | | A₃ | A₃ | | | | | | | | | | | A₂ | A₂ | A₂ | | A₂ | A₂ | B₇ | | |
| | | | A_{1b} | A₂ | | | | | | | | | | | B₇ | B₇ | B₇ | | B₇ | B₇ | B₂₇ | | |

| | |
|---|---|
| B₂₇ | B₄₄ |
| | A₂₄ |

Linker Length in Amino Acids

| Class I MHC | 0 | 1 | 2 | 3 | 4 | 5 | 6 |
|---|---|---|---|---|---|---|---|
| A₂ | 1 | 2 | 2 | 1 | 0 | 0 | 0 |
| A₃ | 0 | 0 | 0 | 0 | 0 | 0 | 0 |
| A_{1b} | 0 | 0 | 0 | 0 | 0 | 0 | 0 |
| Total | 1 | 2 | 2 | 1 | 0 | 0 | 0 |

The 2(N-terminal)-4(C-terminal) epitope pairing can be used in a polyepitope with zero junctional epitopes for the Class I haplotype motifs considered here. Linkers of four, five, or six amino acids in length will produce no junctional epitopes.

To avoid junctional epitopes, the following amino acids MUST be avoided in a four amino acid linker between epitopes 2(N-terminal)-4(C-terminal). The linker is shown from left to right, with the most N-terminal residue of the linker occuring at the left, and the most C-terminal residue occuring to the right. Amino acids to be avoided are shown position by position in the linker.

| | | | | | |
|---|---|---|---|---|---|
| N-End | _{—} | _{—} | _{—} | _{—} | C-End |
| | L | K | K | K | |
| | M | R | R | R | |
| | P | L | V | V | |
| | | M | I | I | |
| | | P | L | L | |
| | | Y | M | P | |
| | | | P | Y | |
| | | | E | E | |
| | | | Y | | |

### Example 14

Junctional epitopes between epitopes 2 and 5 as a function of linker length. Epitope 2 at N-end, Epitope 5 at C-end

| | | | | | | | | | | | | | | | | | | | | | | | | | |
|---|---|---|---|---|---|---|---|---|---|---|---|---|---|---|---|---|---|---|---|---|---|---|---|---|---|
| AKFV | A | A | W | T | L | K | A | A | A | _{—} | _{—} | _{—} | _{—} | _{—} | _{—} | Y | L | Q | L | V | F | G | I | E | V |
| | | | | A₃ | A₂ | | | | | | | | | | | A_{1b} | A₂ | | A₂ | A₂ | B₇ | | A₂ | | |
| | | | | A_{1b} | A₃ | | | | | | | | | | | A_{1c} | B₇ | | B₇ | B₇ | A₂₄ | | B₇ | | |
| | | | | | | | | | | | | | | | | B₄₄ | B₂₇ | | B₂₇ | | B₄₄ | | | | |
| | | | | | | | | | | | | | | | | | | | | | B₂₇ | | | | |

Linker Length in Amino Acids

| Class I MHC | 0 | 1 | 2 | 3 | 4 | 5 | 6 |
|---|---|---|---|---|---|---|---|
| A₂ | 1 | 1 | 1 | 0 | 0 | 0 | 0 |
| A₃ | 0 | 0 | 0 | 0 | 0 | 0 | 0 |
| A_{1B} | 0 | 1 | 1 | 0 | 0 | 0 | 0 |
| Total | 1 | 2 | 2 | 0 | 0 | 0 | 0 |

The 2(N-terminal)-5(C-terminal) epitope pairing can be used in a polyepitope with zero junctional epitopes for the Class I haplotype motifs considered here. Linkers of three, four, five, or six amino acids in length will produce no junctional epitopes.

To avoid junctional epitopes, the following amino acids MUST be avoided in a three amino acid linker between epitopes 2(N-terminal)-5(C-terminal). The linker is shown from left to right, with the most N-terminal residue of the linker occurring at the left, and the most C-terminal residue occurring to the right. Amino acids to be avoided are shown position by position in the linker.

| | | | | |
|---|---|---|---|---|
| N-End | _{—} | _{—} | _{—} | C-End |
| | L | K | K | |
| | M | R | R | |
| | P | Y | Y | |
| | R | P | V | |
| | Y | E | I | |
| | E | | L | |
| | | | M | |
| | | | P | |

### Example 15

Junctional epitopes between epitopes 2 and 6 as a function of linker length. Epitope pair 13: Epitope 2 at N-end, Epitope 6 at C-end

| | | | | | | | | | | | | | | | | | | | | | | | |
|---|---|---|---|---|---|---|---|---|---|---|---|---|---|---|---|---|---|---|---|---|---|---|---|
| AKFVA | A | W | T | L | K | A | A | A | _{—} | _{—} | _{—} | _{—} | _{—} | _{—} | I | M | I | G | V | L | V | G | V |
| | | | A₃ | A₂ | | | | | | | | | | | A₂ | B₇ | A₂ | | A₂ | A₂ | A₂ | | |
| | | | A_{1b} | A₃ | | | | | | | | | | | B₇ | | B₇ | | B₇ | B₇ | B₇ | | |
| | | | | | | | | | | | | | | | | | | | | B₂₇ | | | |

Linker Length in Amino Acids

| Class I MHC | 0 | 1 | 2 | 3 | 4 | 5 | 6 |
|---|---|---|---|---|---|---|---|
| A₂ | 1 | 1 | 1 | 1 | 0 | 0 | 0 |
| A₃ | 0 | 0 | 0 | 0 | 0 | 0 | 0 |
| A_{1B} | 0 | 0 | 0 | 0 | 0 | 0 | 0 |
| Total | 1 | 1 | 1 | 1 | 0 | 0 | 0 |

The 2(N-terminal)-6(C-terminal) epitope pairing can be used in a polyepitope with zero junctional epitopes for the Class I haplotype motifs considered here. Linkers of four, five, or six amino acids in length will produce no junctional epitopes.

To avoid junctional epitopes, the following amino acids MUST be avoided in a four amino acid linker between epitopes 2(N-terminal)-6(C-terminal). The linker is shown from left to right, with the most N-terminal residue of the linker occurring at the left, and the most C-terminal residue occurring to the right. Amino acids to be avoided are shown position by position in the linker.

| | | | | | |
|---|---|---|---|---|---|
| N-End | _{—} | _{—} | _{—} | _{—} | C-End |
| | L | K | V | V | |
| | M | M | I | I | |
| | P | P | L | L | |
| | | R | K | K | |
| | | Y | R | R | |
| | | L | P | P | |
| | | | Y | M | |
| | | | M | | |

### Example 16

Junctional epitopes between epitopes 2 and 7 as a function of linker length. Epitope 2 at N-end, Epitope 7 at C-end

| | | | | | | | | | | | | | | | | | | | | | | | |
|---|---|---|---|---|---|---|---|---|---|---|---|---|---|---|---|---|---|---|---|---|---|---|---|
| AKFVA | A | W | T | L | K | A | A | A | _{—} | _{—} | _{—} | _{—} | _{—} | _{—} | Y | L | S | G | A | N | L | N | V |
| | | | A₃ | A₂ | | | | | | | | | | | A_{1b} | A₂ | | | | | A₂ | | |
| | | | A_{1b} | A₃ | | | | | | | | | | | A_{1c} | B₇ | | | | | B₇ | | |
| | | | | | | | | | | | | | | | B₄₄ | B₂₇ | | | | | B₂₇ | | |

Linker Length in Amino Acids

| Class I MHC | 0 | 1 | 2 | 3 | 4 | 5 | 6 |
|---|---|---|---|---|---|---|---|
| A₂ | 0 | 1 | 1 | 0 | 0 | 0 | 0 |
| A₃ | 0 | 0 | 0 | 0 | 0 | 0 | 0 |
| A_{1B} | 0 | 1 | 1 | 0 | 0 | 0 | 0 |
| Total | 0 | 2 | 2 | 0 | 0 | 0 | 0 |

The 2(N-terminal)-7(C-terminal) epitope pairing can be used in a polyepitope with zero junctional epitopes for the Class I haplotype motifs considered here. Linkers of zero, three, four, five, or six amino acids in length will produce no junctional epitopes. As there are no amino acids in a zero amino acid linker, there are no constraints on the amino acids used for a zero length linker.

### Example 17

Junctional epitopes between epitopes 2 and 8 as a function of linker length. Epitope 2 at N-end, Epitope 8 at C-end

| | | | | | | | | | | | | | | | | | | | | | | | |
|---|---|---|---|---|---|---|---|---|---|---|---|---|---|---|---|---|---|---|---|---|---|---|---|
| AKFVA | A | W | T | L | K | A | A | A | _{—} | _{—} | _{—} | _{—} | _{—} | _{—} | R | L | L | Q | E | T | E | L | V |
| | | | A₃ | A₂ | | | | | | | | | | | A₃ | A₂ | A₂ | | | | | A₂ | |
| | | | A_{1b} | A₃ | | | | | | | | | | | B₂₇ | B₇ | B₇ | | | | | B₇ | |
| | | | | | | | | | | | | | | | | B₂₇ | B₂₇ | | | | | B₂₇ | |

Linker Length in Amino Acids

| Class I MHC | 0 | 1 | 2 | 3 | 4 | 5 | 6 |
|---|---|---|---|---|---|---|---|
| A₂ | 1 | 2 | 1 | 0 | 0 | 0 | 0 |
| A₃ | 0 | 1 | 2 | 1 | 0 | 0 | 0 |
| A_{1b} | 0 | 0 | 0 | 0 | 0 | 0 | 0 |
| Total | 1 | 3 | 3 | 1 | 0 | 0 | 0 |

The 2(N-terminal)-8(C-terminal) epitope pairing can be used in a polyepitope with zero junctional epitopes for the Class I haplotype motifs considered here. Linkers of four, five, or six amino acids in length will produce no junctional epitopes.

To avoid junctional epitopes, the following amino acids MUST be avoided in a four amino acid linker between epitopes 2(N-terminal)-8(C-terminal). The linker is shown from left to right, with the most N-terminal residue of the linker occuring at the left, and the most C-terminal residue occuring to the right. Amino acids to be avoided are shown position by position in the linker.

| | | | | | |
|---|---|---|---|---|---|
| N-End | _{—} | _{—} | _{—} | _{—} | C-End |
| | | K | K | V | |
| | | R | R | I | |
| | | Y | Y | M | |
| | | | V | L | |
| | | | I | R | |
| | | | L | K | |
| | | | | P | |

### Example 18

Junctional epitopes between epitopes 2 and 9 as a function of linker length. Epitope 2 at N-end, Epitope 9 at C-end

| | | | | | | | | | | | | | | | | | | | | | | | |
|---|---|---|---|---|---|---|---|---|---|---|---|---|---|---|---|---|---|---|---|---|---|---|---|
| AKFVA | A | W | T | L | K | A | A | A | _{—} | _{—} | _{—} | _{—} | _{—} | _{—} | S | M | P | P | P | G | T | R | V |
| | | | A₃ | A₂ | | | | | | | | | | | | B₇ | | | | | | B₂₇ | |
| | | | A_{1b} | A₃ | | | | | | | | | | | | | | | | | | A₃ | |

Linker Length in Amino Acids

| Class I MHC | 0 | 1 | 2 | 3 | 4 | 5 | 6 |
|---|---|---|---|---|---|---|---|
| A₂ | 0 | 0 | 0 | 0 | 0 | 0 | 0 |
| A₃ | 0 | 0 | 0 | 0 | 0 | 0 | 0 |
| A_{1b} | 0 | 0 | 0 | 0 | 0 | 0 | 0 |
| Total | 0 | 0 | 0 | 0 | 0 | 0 | 0 |

The 2(N-terminal)-9(C-terminal) epitope pairing can be used in a polyepitope with zero junctional epitopes for the Class I haplotype motifs considered here. Linkers of zero amino acids in length will produce no junctional epitopes. As there are no amino acids in a zero amino acid linker, there are no constraints on the amino acids used.

### Example 19

Junctional epitopes between epitopes 3 and 1 as a function of linker length. Epitope 3 at N-end, Epitope 1 at C-end

| | | | | | | | | | | | | | | | | | | | | | | | |
|---|---|---|---|---|---|---|---|---|---|---|---|---|---|---|---|---|---|---|---|---|---|---|---|
| K | V | A | E | L | V | H | F | L | _{—} | _{—} | _{—} | _{—} | _{—} | _{—} | K | L | C | P | V | Q | L | W | V |
| | A₃ | | B₄₄ | A₂ | A₃ | | | A₂ | | | | | | | A₃ | A₂ | | | A₂ | | A₂ | | |
| | | | A_{1c} | A₃ | | | | A₃ | | | | | | | B₂₇ | B₇ | | | B₇ | | B₇ | | |
| | | | | | | | | | | | | | | | | B₂₇ | | | | | B₂₇ | | |

Linker Length in Amino Acids

| Class I MHC | 0 | 1 | 2 | 3 | 4 | 5 | 6 |
|---|---|---|---|---|---|---|---|
| A_{1c} | 0 | 0 | 0 | 0 | 0 | 0 | 0 |
| A₃ | 1 | 0 | 1 | 2 | 1 | 0 | 1 |
| B₄₄ | 0 | 0 | 0 | 0 | 0 | 0 | 0 |
| A₂ | 1 | 2 | 2 | 1 | 0 | 1 | 1 |
| Total | 2 | 2 | 3 | 3 | 1 | 1 | 2 |

Allowing linkers of 0 to 6 amino acids between epitopes, the 3(N-terminal)-1 (C-terminal) epitope pairing cannot be used in a polyepitope with zero junctional epitopes for the Class I haplotype motifs considered here.

### Example 20

Junctional epitopes between epitopes 3 and 2 as a function of linker length. Epitope 3 at N-end, Epitope 2 at C-end

| | | | | | | | | | | | | | | | | | | | | | | | |
|---|---|---|---|---|---|---|---|---|---|---|---|---|---|---|---|---|---|---|---|---|---|---|---|
| K | V | A | E | L | V | H | F | L | _{—} | _{—} | _{—} | _{—} | _{—} | _{—} | A | K | F | V | AA | W | T | L | KAAA |
| | A₃ | | B₄₄ | A₂ | A₃ | | | A₂ | | | | | | | | A₃ | B₇ | A₂ | | | | | |
| | | | A_{1c} | A₃ | | | | A₃ | | | | | | | | B₂₇ | B₂₇ | B₇ | | | | | |
| | | | | | | | | | | | | | | | | | B₄₄ | | | | | | |
| | | | | | | | | | | | | | | | | | A₂₄ | | | | | | |

Linker Length in Amino Acids

| Class I MHC | 0 | 1 | 2 | 3 | 4 | 5 | 6 |
|---|---|---|---|---|---|---|---|
| A_{1c} | 0 | 0 | 0 | 0 | 0 | 0 | 0 |
| A₃ | 0 | 1 | 2 | 1 | 0 | 1 | 1 |
| B₄₄ | 1 | 0 | 0 | 0 | 0 | 0 | 0 |
| A₂ | 1 | 0 | 0 | 1 | 1 | 0 | 0 |
| Total | 2 | 1 | 2 | 2 | 1 | 1 | 1 |

Allowing linkers of 0 to 6 amino acids between epitopes, the 3(N-terminal)-2 (C-terminal) epitope pairing cannot be used in a polyepitope with zero junctional epitopes for the Class I haplotype motifs considered here.

### Example 21

Junctional epitopes between epitopes 3 and 3 as a function of linker length. Epitope 3 at N-end, Epitope 3 at C-end

| | | | | | | | | | | | | | | | | | | | | | | | |
|---|---|---|---|---|---|---|---|---|---|---|---|---|---|---|---|---|---|---|---|---|---|---|---|
| K | V | A | E | L | V | H | F | L | _{—} | _{—} | _{—} | _{—} | _{—} | _{—} | K | V | A | E | L | V | H | F | L |
| | A₃ | | B₄₄ | A₂ | A₃ | | | A₂ | | | | | | | A₃ | A₂ | | | A₂ | A₂ | | B₇ | |
| | | | A_{1c} | A₃ | | | | A₃ | | | | | | | B₂₇ | B₇ | | | B₇ | B₇ | | B₂₇ | |
| | | | | | | | | | | | | | | | | | | | B₂₇ | | | A₂₄ | |
| | | | | | | | | | | | | | | | | | | | | | | B₄₄ | |

Linker Length in Amino Acids

| Class I MHC | 0 | 1 | 2 | 3 | 4 | 5 | 6 |
|---|---|---|---|---|---|---|---|
| A_{1c} | 0 | 0 | 0 | 0 | 0 | 0 | 0 |
| A₃ | 1 | 0 | 1 | 2 | 1 | 0 | 1 |
| B₄₄ | 0 | 0 | 0 | 0 | 0 | 0 | 0 |
| A₂ | 0 | 2 | 3 | 1 | 0 | 1 | 1 |
| Total | 1 | 2 | 4 | 3 | 1 | 1 | 2 |

Allowing linkers of 0 to 6 amino acids between epitopes, the 3(N-terminal)-2 (C-terminal) epitope pairing cannot be used in a polyepitope with zero junctional epitopes for the Class I haplotype motifs considered here.

### Example 22

Junctional epitopes between epitopes 3 and 4 as a function of linker length. Epitope 3 at N-end, Epitope 4 at C-end

| | | | | | | | | | | | | | | | | | | | | | | | |
|---|---|---|---|---|---|---|---|---|---|---|---|---|---|---|---|---|---|---|---|---|---|---|---|
| K | V | A | E | L | V | H | F | L | _{—} | _{—} | _{—} | _{—} | _{—} | _{—} | V | V | L | G | V | V | F | G | I |
| | A₃ | | B₄₄ | A₂ | A₃ | | | A₂ | | | | | | | A₂ | A₂ | A₂ | | A₂ | A₂ | B₇ | | |
| | | | A_{1c} | A₃ | | | | A₃ | | | | | | | B₇ | B₇ | B₇ | | B₇ | B₇ | B₄₄ | | |

| | |
|---|---|
| B₂₇ | B₂₇ |
| | A₂₄ |

Linker Length in Amino Acids

| Class I MHC | 0 | 1 | 2 | 3 | 4 | 5 | 6 |
|---|---|---|---|---|---|---|---|
| A_{1c} | 0 | 0 | 0 | 0 | 0 | 0 | 0 |
| A₃ | 0 | 0 | 0 | 0 | 0 | 0 | 0 |
| B₄₄ | 0 | 0 | 0 | 0 | 0 | 0 | 0 |
| A₂ | 1 | 3 | 4 | 2 | 1 | 2 | 2 |
| Total | 1 | 3 | 4 | 2 | 1 | 2 | 2 |

Allowing linkers of 0 to 6 amino acids between epitopes, the 3(N-terminal)-4 (C-terminal) epitope pairing cannot be used in a polyepitope with zero junctional epitopes for the Class I haplotype motifs considered here.

### Example 23

Junctional epitopes between epitopes 3 and 5 as a function of linker length. Epitope 3 at N end, Epitope 5 at C end

| | | | | | | | | | | | | | | | | | | | | | | | | |
|---|---|---|---|---|---|---|---|---|---|---|---|---|---|---|---|---|---|---|---|---|---|---|---|---|
| K | V | A | E | L | V | H | F | L | _{—} | _{—} | _{—} | _{—} | _{—} | _{—} | Y | L | Q | L | V | F | G | I | E | V |
| | A₃ | | B₄₄ | A₂ | A₃ | | | A₂ | | | | | | | A_{1b} | A₂ | | A₂ | A₂ | A₂₄ | | A₂ | | |
| | | | A_{1c} | A₃ | | | | A₃ | | | | | | | A_{1c} | B₇ | | B₇ | B₇ | B₇ | | B₇ | | |
| | | | | | | | | | | | | | | | B₄₄ | B₂₇ | | B₂₇ | | B₄₄ | | | | |
| | | | | | | | | | | | | | | | | | | | | B₂₇ | | | | |

Linker Length in Amino Acids

| Class I MHC | 0 | 1 | 2 | 3 | 4 | 5 | 6 |
|---|---|---|---|---|---|---|---|
| A₃ | 0 | 0 | 0 | 0 | 0 | 0 | 0 |
| B₄₄ | 0 | 1 | 1 | 0 | 0 | 0 | 0 |
| A_{1c} | 1 | 1 | 0 | 0 | 0 | 0 | 0 |
| A₂ | 2 | 1 | 2 | 2 | 1 | 1 | 1 |
| Total | 3 | 3 | 3 | 2 | 1 | 1 | 1 |

Allowing linkers of 0 to 6 amino acids between epitopes, the 3(N-terminal)-5 (C-terminal) epitope pairing cannot be used in a polyepitope with zero junctional epitopes for the Class I haplotype motifs considered here.

### Example 24

Junctional epitopes between epitopes 3 and 6 as a function of linker length. Epitope 3 at N-end, Epitope 6 at C-end

| | | | | | | | | | | | | | | | | | | | | | | | |
|---|---|---|---|---|---|---|---|---|---|---|---|---|---|---|---|---|---|---|---|---|---|---|---|
| K | V | A | E | L | V | H | F | L | _{—} | _{—} | _{—} | _{—} | _{—} | _{—} | I | M | I | G | V | L | V | G | V |
| | A₃ | | B₄₄ | A₂ | A₃ | | | A₂ | | | | | | | A₂ | B₇ | A₂ | | A₂ | A₂ | A₂ | | |
| | | | A_{1c} | A₃ | | | | A₃ | | | | | | | B₇ | | B₇ | | B₇ | B₇ | B₇ | | |
| | | | | | | | | | | | | | | | | | | | | B₂₇ | | | |

Linker Length in Amino Acids

| Class I MHC | 0 | 1 | 2 | 3 | 4 | 5 | 6 |
|---|---|---|---|---|---|---|---|
| A₃ | 0 | 0 | 0 | 0 | 0 | 0 | 0 |
| B₄₄ | 0 | 0 | 0 | 0 | 0 | 0 | 0 |
| A_{1c} | 0 | 0 | 0 | 0 | 0 | 0 | 0 |
| A₂ | 2 | 3 | 3 | 2 | 1 | 1 | 1 |
| Total | 2 | 3 | 3 | 2 | 1 | 1 | 1 |

Allowing linkers of 0 to 6 amino acids between epitopes, the 3(N-terminal)-6 (C-terminal) epitope pairing cannot be used in a polyepitope with zero junctional epitopes for the Class I haplotype motifs considered here.

### Example 25

Junctional epitopes between epitopes 3 and 7 as a function of linker length. Epitope 3 at N-end, Epitope 7 at C-end

| | | | | | | | | | | | | | | | | | | | | | | | |
|---|---|---|---|---|---|---|---|---|---|---|---|---|---|---|---|---|---|---|---|---|---|---|---|
| K | V | A | E | L | V | H | F | L | _{—} | _{—} | _{—} | _{—} | _{—} | _{—} | Y | L | S | G | A | N | L | N | V |
| | A₃ | | B₄₄ | A₂ | A₃ | | | A₂ | | | | | | | A_{1b} | A₂ | | | | | A₂ | | |
| | | | A_{1c} | A₃ | | | | A₃ | | | | | | | A_{1c} | B₇ | | | | | B₇ | | |
| | | | | | | | | | | | | | | | B₄₄ | B₂₇ | | | | | B₂₇ | | |

Linker Length in Amino Acids

| Class I MHC | 0 | 1 | 2 | 3 | 4 | 5 | 6 |
|---|---|---|---|---|---|---|---|
| A_{1c} | 1 | 1 | 0 | 0 | 0 | 0 | 0 |
| A₃ | 0 | 0 | 0 | 0 | 0 | 0 | 0 |
| B₄₄ | 0 | 1 | 1 | 0 | 0 | 0 | 0 |
| A₂ | 1 | 2 | 1 | 0 | 0 | 0 | 0 |
| Total | 2 | 4 | 2 | 0 | 0 | 0 | 0 |

The 3(N-terminal)-7(C-terminal) epitope pairing can be used in a polyepitope with zero junctional epitopes for the Class I haplotypes considered here. Linkers of three or four amino acids in length will produce no junctional epitopes.

To avoid junctional epitopes, the following amino acids MUST be avoided in a three amino acid linker between epitopes 3(N-terminal)-7(C-terminal). The linker is shown from left to right, with the most N-terminal residue of the linker occuring at the left, and the most C-terminal residue occuring to the right. Amino acids to be avoided are shown position by position in the linker.

| | | | | |
|---|---|---|---|---|
| N-End | _{—} | _{—} | _{—} | C-End |
| | K | Y | K | |
| | R | F | R | |
| | Y | L | F | |
| | | M | Y | |
| | | P | L | |
| | | R | M | |
| | | | P | |
| | | | V | |
| | | | I | |

### Example 26

Junctional epitopes between epitopes 3 and 8 as a function of linker length. Epitope 3 at N-end, Epitope 8 at C-end

| | | | | | | | | | | | | | | | | | | | | | | | |
|---|---|---|---|---|---|---|---|---|---|---|---|---|---|---|---|---|---|---|---|---|---|---|---|
| K | V | A | E | L | V | H | F | L | _{—} | _{—} | _{—} | _{—} | _{—} | _{—} | R | L | L | Q | E | T | E | L | V |
| | A₃ | | B₄₄ | A₂ | A₃ | | | A₂ | | | | | | | A₃ | A₂ | A₂ | | | | | A₂ | |
| | | | A_{1c} | A₃ | | | | A₃ | | | | | | | B₂₇ | B₇ | B₇ | | | | | B₇ | |
| | | | | | | | | | | | | | | | | B₂₇ | B₂₇ | | | | | B₂₇ | |

Linker Length in Amino Acids

| Class I MHC | 0 | 1 | 2 | 3 | 4 | 5 | 6 |
|---|---|---|---|---|---|---|---|
| A_{1c} | 0 | 0 | 0 | 0 | 0 | 0 | 0 |
| A₃ | 1 | 0 | 1 | 2 | 1 | 0 | 1 |
| B₄₄ | 0 | 0 | 0 | 0 | 0 | 0 | 0 |
| A₂ | 2 | 2 | 1 | 0 | 1 | 2 | 1 |
| Total | 3 | 2 | 2 | 2 | 2 | 2 | 2 |

Allowing linkers of 0 to 6 amino acids between epitopes, the 3(N-terminal)-8 (C-terminal) epitope pairing cannot be used in a polyepitope with zero junctional epitopes for the Class I haplotype motifs considered here

### Example 27

Junctional epitopes between epitopes 3 and 9 as a function of linker length. Epitope pair 24: Epitope 3 at N-end, Epitope 9 at C-end

| | | | | | | | | | | | | | | | | | | | | | | | |
|---|---|---|---|---|---|---|---|---|---|---|---|---|---|---|---|---|---|---|---|---|---|---|---|
| K | V | A | E | L | V | H | F | L | _{—} | _{—} | _{—} | _{—} | _{—} | _{—} | S | M | P | P | P | G | T | R | V |
| | A₃ | | B₄₄ | A₂ | A₃ | | | A₂ | | | | | | | | B₇ | | | | | | A₃ | |
| | | | A_{1c} | A₃ | | | | A₃ | | | | | | | | | | | | | | B₂₇ | |

Linker Length in Amino Acids

| Class I MHC | 0 | 1 | 2 | 3 | 4 | 5 | 6 |
|---|---|---|---|---|---|---|---|
| A_{1c} | 0 | 0 | 0 | 0 | 0 | 0 | 0 |
| A₃ | 1 | 0 | 0 | 0 | 0 | 0 | 0 |
| B₄₄ | 0 | 0 | 0 | 0 | 0 | 0 | 0 |
| A₂ | 0 | 0 | 0 | 0 | 0 | 0 | 0 |
| Total | 1 | 0 | 0 | 0 | 0 | 0 | 0 |

The 3(N-terminal)-9(C-terminal) epitope pairing can be used in a polyepitope with zero junctional epitopes for the Class I haplotype motifs considered here. Linkers of one, two, three, four, five, or six amino acids in length will produce no junctional epitopes.

To avoid junctional epitopes, the following amino acids MUST be avoided in a one amino acid linker between epitopes 3(N-terminal)-9(C-terminal). The linker is shown from left to right, with the most N-terminal residue of the linker occuring at the left, and the most C-terminal residue occuring to the right. Amino acids to be avoided are shown position by position in the linker.

| | | |
|---|---|---|
| N End | _{—} | C End |
| | Y | |
| | K | |
| | R | |
| | L | |
| | M | |
| | V | |
| | T | |

### Example 28

Junctional epitopes between epitopes 4 and 1 as a function of linker length. Epitope 4 at N-end, Epitope 1 at C-end

| | | | | | | | | | | | | | | | | | | | | | | | |
|---|---|---|---|---|---|---|---|---|---|---|---|---|---|---|---|---|---|---|---|---|---|---|---|
| V | V | L | G | V | V | F | G | I | _{—} | _{—} | _{—} | _{—} | _{—} | _{—} | K | L | C | P | V | Q | L | W | V |
| | A₃ | A₂ | | A₃ | A₃ | | | | | | | | | | A₃ | A₂ | | | A₂ | | A₂ | | |
| | | A₃ | | | | | | | | | | | | | B₂₇ | B₇ | | | B₇ | | B₇ | | |
| | | | | | | | | | | | | | | | | B₂₇ | | | | | B₂₇ | | |

Linker Length in Amino Acids

| Class I MHC | 0 | 1 | 2 | 3 | 4 | 5 | 6 |
|---|---|---|---|---|---|---|---|
| A₃ | 2 | 1 | 1 | 2 | 1 | 0 | 0 |
| A₂ | 1 | 0 | 0 | 0 | 0 | 0 | 0 |
| Total | 3 | 1 | 1 | 2 | 1 | 0 | 0 |

To avoid junctional epitopes, the following amino acids MUST be avoided in a five or six amino acid linker between epitopes 4(N-terminal)-1(C-terminal). The linker is shown from left to right, with the most N-terminal residue of the linker occuring at the left, and the most C-terminal residue occuring to the right. Amino acids to be avoided are shown position by position in the linker

| | | | | | | |
|---|---|---|---|---|---|---|
| N-End | _{—} | _{—} | _{—} | _{—} | _{—} | C-End |
| | V | V | K | K | K | |
| | I | I | R | R | R | |
| | L | L | L | L | L | |
| | K | K | M | M | M | |
| | R | R | P | P | P | |
| | | M | | | | |
| | | P | | | | |

### Example 29

Junctional epitopes between epitopes 4 and 2 as a function of linker length. Epitope pair 26: Epitope 4 at N-end, Epitope 2 at C-end

| | | | | | | | | | | | | | | | | | | | | | | | | | | |
|---|---|---|---|---|---|---|---|---|---|---|---|---|---|---|---|---|---|---|---|---|---|---|---|---|---|---|
| V | V | L | G | V | V | F | G | I | _{—} | _{—} | _{—} | _{—} | _{—} | _{—} | A | K | F | V | A | A | W | T | L | K | A | AA |
| | A₃ | A₂ | | A₃ | A₃ | | | | | | | | | | | A₃ | B₄₄ | B₇ | | | | | | | | |
| | | A₃ | | | | | | | | | | | | | | B₂₇ | B₇ | A₂ | | | | | | | | |
| | | | | | | | | | | | | | | | | | A₂₄ | | | | | | | | | |
| | | | | | | | | | | | | | | | | | B₂₇ | | | | | | | | | |

Linker Length in Amino Acids

| Class I MHC | 0 | 1 | 2 | 3 | 4 | 5 | 6 |
|---|---|---|---|---|---|---|---|
| A₃ | 1 | 1 | 2 | 1 | 0 | 0 | 0 |
| A₂ | 0 | 0 | 0 | 0 | 0 | 0 | 0 |
| Total | 1 | 1 | 2 | 1 | 0 | 0 | 0 |

### Example 29

### Extension

The 4(N-terminal)-2(C-terminal) epitope pairing can be used in a polyepitope with zero junctional epitopes for the Class I haplotype motifs considered here. Linkers of four, five, or six amino acids in length will produce no junctional epitopes.

To avoid junctional epitopes, the following amino acids MUST be avoided in a four amino acid linker between epitopes 4(N-terminal)-2(C-terminal). The linker is shown from left to right, with the most N-terminal residue of the linker occuring at the left, and the most C-terminal residue occuring to the right. Amino acids to be avoided are shown position by position in the linker

| | | | | | | |
|---|---|---|---|---|---|---|
| N-End | _{—} | _{—} | _{—} | _{—} | _{—} | C-End |
| | V | V | K | K | K | |
| | I | I | R | R | R | |
| | L | L | L | | | |
| | K | K | | | | |
| | R | R | | | | |
| | M | | | | | |
| | P | | | | | |

### Example 30

Junctional epitopes between epitopes 4 and 3 as a function of linker length. Epitope 4 at N-end, Epitope 3 at C-end

| | | | | | | | | | | | | | | | | | | | | | | | |
|---|---|---|---|---|---|---|---|---|---|---|---|---|---|---|---|---|---|---|---|---|---|---|---|
| V | V | L | G | V | V | F | G | I | _{—} | _{—} | _{—} | _{—} | _{—} | _{—} | K | V | A | E | L | V | H | F | L |
| | A₃ | A₂ | | A₃ | A₃ | | | | | | | | | | A₃ | A₂ | | | A₂ | A₂ | | B₇ | |
| | | A₃ | | | | | | | | | | | | | B₂₇ | B₇ | | | B₇ | B₇ | | A₂₄ | |
| | | | | | | | | | | | | | | | | | | | B₂₇ | | | B₂₇ | |
| | | | | | | | | | | | | | | | | | | | | | | B₄₄ | |

Linker Length in Amino Acids

| Class I MHC | 0 | 1 | 2 | 3 | 4 | 5 | 6 |
|---|---|---|---|---|---|---|---|
| A₃ | 2 | 1 | 1 | 2 | 1 | 0 | 0 |
| A₂ | 1 | 0 | 0 | 0 | 0 | 0 | 0 |
| Total | 3 | 1 | 1 | 2 | 1 | 0 | 0 |

The 4(N-terminal)-3(C-terminal) epitope pairing can be used in a polyepitope with zero junctional epitopes for the Class I haplotype motifs considered here. Linkers of five or six amino acids in length will produce no junctional epitopes.

To avoid junctional epitopes, the following amino acids MUST be avoided in a five amino acid linker between epitopes 4(N-terminal)-3(C-terminal). The linker is shown from left to right, with the most N-terminal residue of the linker occuring at the left, and the most C-terminal residue occuring to the right. Amino acids to be avoided are shown position by position in the linker.

| | | | | | | |
|---|---|---|---|---|---|---|
| N-End | _{—} | _{—} | _{—} | _{—} | _{—} | C-End |
| | K | K | K | K | K | |
| | R | R | R | R | R | |
| | V | V | M | L | P | |
| | I | I | L | M | Y | |
| | L | L | P | P | E | |
| | | M | | | | |
| | | P | | | | |

### Example 31

Junctional epitopes between epitopes 4 and 4 as a function of linker length. Epitope 4 at N-end, Epitope 4 at C-end

| | | | | | | | | | | | | | | | | | | | | | | | |
|---|---|---|---|---|---|---|---|---|---|---|---|---|---|---|---|---|---|---|---|---|---|---|---|
| V | V | L | G | V | V | F | G | I | _{—} | _{—} | _{—} | _{—} | _{—} | _{—} | V | V | L | G | V | V | F | G | I |
| | A₃ | A₂ | | A₃ | A₃ | | | | | | | | | | A₂ | A₂ | A₂ | | A₂ | A₂ | B₇ | | |
| | | A₃ | | | | | | | | | | | | | B₇ | B₇ | B₇ | | B₇ | B₇ | B₄₄ | | |
| | | | | | | | | | | | | | | | | | B₂₇ | | | | B₂₇ | | |
| | | | | | | | | | | | | | | | | | | | | | A₂₄ | | |

Linker Length in Amino Acids

| Class I MHC | 0 | 1 | 2 | 3 | 4 | 5 | 6 |
|---|---|---|---|---|---|---|---|
| A₃ | 0 | 0 | 0 | 0 | 0 | 0 | 0 |
| A₂ | 2 | 1 | 0 | 0 | 0 | 0 | 0 |
| Total | 2 | 1 | 0 | 0 | 0 | 0 | 0 |

The 4(N-terminal)-4(C-terminal) epitope pairing can be used in a polyepitope with zero junctional epitopes for the Class I haplotype motifs considered here. Linkers of two, three, four, five, or six amino acids in length will produce no junctional epitopes.

To avoid junctional epitopes, the following amino acids MUST be avoided in a two amino acid linker between epitopes 4(N-terminal)-4(C-terminal). The linker is shown from left to right, with the most N-terminal residue of the linker occuring at the left, and the most C-terminal residue occuring to the right. Amino acids to be avoided are shown position by position in the linker.

| | | | |
|---|---|---|---|
| N-End | _{—} | _{—} | C-End |
| | V | V | |
| | I | I | |
| | L | L | |
| | K | K | |
| | R | R | |
| | M | P | |
| | P | Y | |
| | Y | E | |
| | E | | |

### Example 32

Junctional epitopes between epitopes 4 and 5 as a function of linker length. Epitope 4 at N end, Epitope 5 at C end

| | | | | | | | | | | | | | | | | | | | | | | | | |
|---|---|---|---|---|---|---|---|---|---|---|---|---|---|---|---|---|---|---|---|---|---|---|---|---|
| V | V | L | G | V | V | F | G | I | _{—} | _{—} | _{—} | _{—} | _{—} | _{—} | Y | L | Q | L | V | F | G | I | E | V |
| | A₃ | A₂ | | A₃ | A₃ | | | | | | | | | | A_{1c} | A₂ | | A₂ | A₂ | B₇ | | A₂ | | |
| | | A₃ | | | | | | | | | | | | | A_{1b} | B₇ | | B₇ | B₇ | A₂₄ | | B₇ | | |
| | | | | | | | | | | | | | | | B₄₄ | B₂₇ | | B₂₇ | | B₄₄ | | | | |
| | | | | | | | | | | | | | | | | | | | | B₂₇ | | | | |

Linker Length in Amino Acids

| Class I MHC | 0 | 1 | 2 | 3 | 4 | 5 | 6 |
|---|---|---|---|---|---|---|---|
| A₃ | 0 | 0 | 0 | 0 | 0 | 0 | 0 |
| A₂ | 1 | 0 | 0 | 0 | 0 | 0 | 0 |
| Total | 1 | 0 | 0 | 0 | 0 | 0 | 0 |

The 4(N-terminal)-5(C-terminal) epitope pairing can be used in a polyepitope with zero junctional epitopes for the Class I haplotype motifs considered here. Linkers of one, two, three, four, five, or six amino acids in length will produce no junctional epitopes.

To avoid junctional epitopes, the following amino acids MUST be avoided in a one amino acid linker between epitopes 4(N-terminal)-5(C-terminal). The linker is shown from left to right, with the most N-terminal residue of the linker occuring at the left, and the most C-terminal residue occuring to the right. Amino acids to be avoided are shown position by position in the linker.

| | | |
|---|---|---|
| N-End | _{—} | C-End |
| | K | |
| | R | |
| | L | |
| | M | |
| | P | |
| | V | |
| | I | |

### Example 33

Junctional epitopes between epitopes 4 and 6 as a function of linker length. Epitope 4 at N-end, Epitope 6 at C-end

| | | | | | | | | | | | | | | | | | | | | | | | |
|---|---|---|---|---|---|---|---|---|---|---|---|---|---|---|---|---|---|---|---|---|---|---|---|
| V | V | L | G | V | V | F | G | I | _{—} | _{—} | _{—} | _{—} | _{—} | _{—} | I | M | I | G | V | L | V | G | V |
| | A₃ | A₂ | | A₃ | A₃ | | | | | | | | | | A₂ | B₇ | A₂ | | A₂ | A₂ | A₂ | | |
| | | A₃ | | | | | | | | | | | | | B₇ | | B₇ | | B₇ | B₇ | B₇ | | |
| | | | | | | | | | | | | | | | | | | | | B₂₇ | | | |

Linker Length in Amino Acids

| Class I MHC | 0 | 1 | 2 | 3 | 4 | 5 | 6 |
|---|---|---|---|---|---|---|---|
| A₃ | 0 | 0 | 0 | 0 | 0 | 0 | 0 |
| A₂ | 1 | 1 | 0 | 0 | 0 | 0 | 0 |
| Total | 1 | 1 | 0 | 0 | 0 | 0 | 0 |

The 4(N-terminal)-6(C-terminal) epitope pairing can be used in a polyepitope with zero junctional epitopes for the Class I haplotype motifs considered here. Linkers of two, three, four, five, or six amino acids in length will produce no junctional epitopes.

To avoid junctional epitopes, the following amino acids MUST be avoided in a two amino acid linker between epitopes 4(N-terminal)-6(C-terminal). The linker is shown from left to right, with the most N-terminal residue of the linker occurring at the left, and the most C-terminal residue occurring to the right. Amino acids to be avoided are shown position by position in the linker.

| | | | |
|---|---|---|---|
| N-End | _{—} | _{—} | C-End |
| | K | K | |
| | R | R | |
| | L | V | |
| | M | I | |
| | P | L | |
| | V | P | |
| | I | M | |

### Example 34

Junctional epitopes between epitopes 4 and 7 as a function of linker length. Epitope 4 at N-end, Epitope 7 at C-end

| | | | | | | | | | | | | | | | | | | | | | | | |
|---|---|---|---|---|---|---|---|---|---|---|---|---|---|---|---|---|---|---|---|---|---|---|---|
| V | V | L | G | V | V | F | G | I | _{—} | _{—} | _{—} | _{—} | _{—} | _{—} | Y | L | S | G | A | N | L | N | V |
| | A₃ | A₂ | | A₃ | A₃ | | | | | | | | | | A_{1b} | A₂ | | | | | A₂ | | |
| | | A₃ | | | | | | | | | | | | | A_{1c} | B₇ | | | | | B₇ | | |
| | | | | | | | | | | | | | | | B₄₄ | B₂₇ | | | | | B₂₇ | | |

Linker Length in Amino Acids

| Class I MHC | 0 | 1 | 2 | 3 | 4 | 5 | 6 |
|---|---|---|---|---|---|---|---|
| A₃ | 0 | 0 | 0 | 0 | 0 | 0 | 0 |
| A₂ | 1 | 0 | 0 | 0 | 0 | 0 | 0 |
| Total | 1 | 0 | 0 | 0 | 0 | 0 | 0 |

The 4(N-terminal)-7(C-terminal) epitope pairing can be used in a polyepitope with zero junctional epitopes for the Class I haplotype motifs considered here. Linkers of one, two, three, four, five, or six amino acids in length will produce no junctional epitopes.

To avoid junctional epitopes, the following amino acids MUST be avoided in a one amino acid linker between epitopes 4(N-terminal)-7(C-terminal). The linker is shown from left to right, with the most N-terminal residue of the linker occuring at the left, and the most C-terminal residue occuring to the right. Amino acids to be avoided are shown position by position in the linker.

| | | |
|---|---|---|
| N-End | _{—} | C-End |
| | K | |
| | R | |
| | L | |
| | M | |
| | P | |
| | V | |
| | I | |

### Example 35

Junctional epitopes between epitopes 4 and 8 as a function of linker length. Epitope 4 at N-end, Epitope 8 at C-end

| | | | | | | | | | | | | | | | | | | | | | | | |
|---|---|---|---|---|---|---|---|---|---|---|---|---|---|---|---|---|---|---|---|---|---|---|---|
| V | V | L | G | V | V | F | G | I | _{—} | _{—} | _{—} | _{—} | _{—} | _{—} | R | L | L | Q | E | T | E | L | V |
| | A₃ | A₂ | | A₃ | A₃ | | | | | | | | | | A₃ | A₂ | A₂ | | | | | A₂ | |
| | | A₃ | | | | | | | | | | | | | B₂₇ | B₇ | B₇ | | | | | B₇ | |
| | | | | | | | | | | | | | | | | B₂₇ | B₂₇ | | | | | B₂₇ | |

Linker Length in Amino Acids

| Class I MHC | 0 | 1 | 2 | 3 | 4 | 5 | 6 |
|---|---|---|---|---|---|---|---|
| A₃ | 2 | 1 | 1 | 2 | 1 | 0 | 0 |
| A₂ | 1 | 0 | 0 | 0 | 0 | 0 | 0 |
| Total | 3 | 1 | 1 | 2 | 1 | 0 | 0 |

The 4(N-terminal)-8(C-terminal) epitope pairing can be used in a polyepitope with zero junctional epitopes for the Class I haplotype motifs considered here. Linkers of five or six amino acids in length will produce no junctional epitopes.

To avoid junctional epitopes, the following amino acids MUST be avoided in a five amino acid linker between epitopes 4(N-terminal)-8(C-terminal). The linker is shown from left to right, with the most N-terminal residue of the linker occuring at the left, and the most C-terminal residue occuring to the right. Amino acids to be avoided are shown position by position-in the linker.

| | | | | | | |
|---|---|---|---|---|---|---|
| N-End | _{—} | _{—} | _{—} | _{—} | _{—} | C-End |
| | K | K | K | K | K | |
| | R | R | R | R | R | |
| | L | V | | | L | |
| | M | I | | | M | |
| | P | L | | | P | |
| | V | | | | | |
| | I | | | | | |

### Example 36

Junctional epitopes between epitopes 4 and 9 as a function of linker length. Epitope 4 at N-end, Epitope 9 at C-end

| | | | | | | | | | | | | | | | | | | | | | | | |
|---|---|---|---|---|---|---|---|---|---|---|---|---|---|---|---|---|---|---|---|---|---|---|---|
| V | V | L | G | V | V | F | G | I | _{—} | _{—} | _{—} | _{—} | _{—} | _{—} | S | M | P | P | P | G | T | R | V |
| | A₃ | A₂ | | A₃ | A₃ | | | | | | | | | | | B₇ | | | | | | A₃ | |
| | | A₃ | | | | | | | | | | | | | | | | | | | | B₂₇ | |

Linker Length in Amino Acids

| Class I MHC | 0 | 1 | 2 | 3 | 4 | 5 | 6 |
|---|---|---|---|---|---|---|---|
| A₃ | 0 | 0 | 0 | 0 | 0 | 0 | 0 |
| A₂ | 0 | 0 | 0 | 0 | 0 | 0 | 0 |
| Total | 0 | 0 | 0 | 0 | 0 | 0 | 0 |

The 4(N-terminal)-9(C-terminal) epitope pairing can be used in a polyepitope with zero junctional epitopes for the Class I haplotype motifs considered here. Linkers of zero, one, two, three, four, five, or six amino acids in length will produce no junctional epitopes. As there are no amino acids in a zero amino acid linker, there are no constraints on the amino acids used in a zero length linker.

### Example 37

Junctional epitopes between epitopes 5 and 1 as a function of linker length. Epitope 5 at N-end, Epitope 1 at C-end

| | | | | | | | | | | | | | | | | | | | | | | | | |
|---|---|---|---|---|---|---|---|---|---|---|---|---|---|---|---|---|---|---|---|---|---|---|---|---|
| Y | L | Q | L | V | F | G | I | E | V | _{—} | _{—} | — | _{—} | _{—} | _{—} | K | L | C | P | V | Q | L | W | V |
| | | | A₂ | | | | | A_{1c} | A₃ | | | | | | | A₃ | A₂ | | | A₂ | | A₂ | | |
| | | | A₃ | A₃ | | | | B₄₄ | | | | | | | | B₂₇ | B₇ | | | B₇ | | B₇ | | |
| | | | | | | | | | | | | | | | | | B₂₇ | | | | | B₂₇ | | |

Linker Length in Amino Acids

| Class I MHC | 0 | 1 | 2 | 3 | 4 | 5 | 6 |
|---|---|---|---|---|---|---|---|
| A3 | 1 | 2 | 1 | 0 | 0 | 0 | 1 |
| A₂ | 1 | 0 | 0 | 0 | 0 | 0 | 0 |
| A_{1c} | 0 | 0 | 0 | 0 | 0 | 0 | 0 |
| B₄₄ | 0 | 0 | 0 | 0 | 0 | 0 | 0 |
| Total | 2 | 2 | 1 | 0 | 0 | 0 | 1 |

The 5(N-terminal)-1(C-terminal) epitope pairing can be used in a polyepitope with zero junctional epitopes for the Class I haplotype motifs considered here. Linkers of three, four, or five amino acids in length will produce no junctional epitopes.

To avoid junctional epitopes, the following amino acids MUST be avoided in a three amino acid linker between epitopes 5(N-terminal)-1(C-terminal). The linker is shown from left to right, with the most N-terminal residue of the linker occurring at the left, and the most C-terminal residue occurring to the right. Amino acids to be avoided are shown position by position in the linker.

| | | | | |
|---|---|---|---|---|
| N-End | _{—} | _{—} | _{—} | C-End |
| | V | V | K | |
| | I | I | R | |
| | L | L | L | |
| | K | K | M | |
| | R | R | P | |
| | M | M | | |
| | P | P | | |

### Example 38

Junctional epitopes between epitopes 5 and 2 as a function of linker length. Epitope 5 at N-end, Epitope 2 at C-end

| | | | | | | | | | | | | | | | | | | | | | | | | | | |
|---|---|---|---|---|---|---|---|---|---|---|---|---|---|---|---|---|---|---|---|---|---|---|---|---|---|---|
| Y | L | Q | L | V | F | G | I | E | V | _{—} | _{—} | _{—} | _{—} | _{—} | _{—} | A | K | F | V | A | A | W | T | L | K | AAA |
| | | | A₂ | | | | | A_{1c} | A₃ | | | | | | | | A₃ | B₇ | A₂ | | | | | | | |
| | | | A₃ | A₃ | | | | B₄₄ | | | | | | | | | B₂₇ | B₄₄ | B₇ | | | | | | | |
| | | | | | | | | | | | | | | | | | | A₂₄ | | | | | | | | |
| | | | | | | | | | | | | | | | | | | A₂₇ | | | | | | | | |

Linker Length in Amino Acids

| Class I MHC | 0 | 1 | 2 | 3 | 4 | 5 | 6 |
|---|---|---|---|---|---|---|---|
| A₂ | 0 | 0 | 0 | 0 | 0 | 0 | 0 |
| A₃ | 2 | 1 | 0 | 0 | 0 | 1 | 1 |
| A1c | 0 | 0 | 0 | 0 | 0 | 0 | 0 |
| B44 | 0 | 0 | 0 | 1 | 1 | 0 | 0 |
| Total | 2 | 1 | 0 | 1 | 1 | 1 | 1 |

The 5(N-terminal)-2(C-terminal) epitope pairing can be used in a polyepitope with zero junctional epitopes for the Class I haplotype motifs considered here. Linkers of two amino acids in length will produce no junctional epitopes.

To avoid junctional epitopes, the following amino acids MUST be avoided in a two amino acid linker between epitopes 5(N-terminal)-2(C-terminal). The linker is shown from left to right, with the most N-terminal residue of the linker occuring at the left, and the most C-terminal residue occuring to the right. Amino acids to be avoided are shown position by position in the linker.

| | | | |
|---|---|---|---|
| N-End | _{—} | _{—} | C-End |
| | V | V | |
| | I | I | |
| | L | L | |
| | K | K | |
| | R | R | |

### Example 39

Junctional epitopes between epitopes 5 and 3 as a function of linker length. Epitope 5 at N end, Epitope 3 at C end

| | | | | | | | | | | | | | | | | | | | | | | | | |
|---|---|---|---|---|---|---|---|---|---|---|---|---|---|---|---|---|---|---|---|---|---|---|---|---|
| Y | L | Q | L | V | F | G | I | E | V | _{—} | _{—} | _{—} | _{—} | _{—} | _{—} | K | V | A | E | L | V | H | F | L |
| | | | A₂ | | | | | A_{1c} | A₃ | | | | | | | B₂₇ | A₂ | | | A₂ | A₂ | | B₇ | |
| | | | A₃ | A₃ | | | | B₄₄ | | | | | | | | A₃ | B₇ | | | B₇ | B₇ | | B₂₇ | |
| | | | | | | | | | | | | | | | | | | | | B₂₇ | | | A₂₄ | |
| | | | | | | | | | | | | | | | | | | | | | | | B₄₄ | |

Linker Length in Amino Acids

| Class I MHC | 0 | 1 | 2 | 3 | 4 | 5 | 6 |
|---|---|---|---|---|---|---|---|
| A₂ | 1 | 0 | 0 | 0 | 0 | 0 | 0 |
| A_{1c} | 0 | 0 | 0 | 0 | 0 | 0 | 0 |
| B₄₄ | 0 | 0 | 0 | 0 | 0 | 0 | 0 |
| A₃ | 1 | 2 | 1 | 0 | 0 | 0 | 1 |
| Total | 2 | 2 | 1 | 0 | 0 | 0 | 1 |

The 5(N-terminal)-3(C-terminal) epitope pairing can be used in a polyepitope with zero junctional epitopes for the Class I haplotype motifs considered here. Linkers of three, four, or five amino acids in length will produce no junctional epitopes.

To avoid junctional epitopes, the following amino acids MUST be avoided in a three amino acid linker between epitopes 5(N-terminal)-3(C-terminal). The linker is shown from left to right, with the most N-terminal residue of the linker occurring at the left, and the most C-terminal residue occurring to the right. Amino acids to be avoided are shown position by position in the linker.

| | | | | |
|---|---|---|---|---|
| N-End | _{—} | _{—} | _{—} | C-End |
| | V | V | K | |
| | I | I | R | |
| | L | L | P | |
| | K | K | Y | |
| | R | R | E | |
| | M | M | | |
| | P | P | | |

### Example 40

Junctional epitopes between epitopes 5 and 4 as a function of linker length. Epitope 5 at N-end, Epitope 4 at C-end

| | | | | | | | | | | | | | | | | | | | | | | | | |
|---|---|---|---|---|---|---|---|---|---|---|---|---|---|---|---|---|---|---|---|---|---|---|---|---|
| Y | L | Q | L | V | F | G | I | E | V | _{—} | _{—} | _{—} | _{—} | _{—} | _{—} | V | V | L | G | V | V | F | G | I |
| | | | A₂ | | | | | A_{1c} | A₃ | | | | | | | A₂ | A₂ | A₂ | | A₂ | A₂ | B₇ | | |
| | | | A₃ | A₃ | | | | B₄₄ | | | | | | | | B₇ | B₇ | B₇ | | B₇ | B₇ | B₂₇ | | |
| | | | | | | | | | | | | | | | | | | B₂₇ | | | | B₄₄ | | |
| | | | | | | | | | | | | | | | | | | | | | | A₂₄ | | |

Linker Length in Amino Acids

| Class I MHC | 0 | 1 | 2 | 3 | 4 | 5 | 6 |
|---|---|---|---|---|---|---|---|
| A₂ | 2 | 1 | 0 | 0 | 0 | 0 | 0 |
| A₃ | 0 | 0 | 0 | 0 | 0 | 0 | 0 |
| A_{1C} | 0 | 0 | 0 | 0 | 0 | 0 | 0 |
| B₄₄ | 1 | 0 | 0 | 0 | 0 | 0 | 0 |
| Total | 3 | 1 | 0 | 0 | 0 | 0 | 0 |

The 5(N-terminal)-4(C-terminal) epitope pairing can be used in a polyepitope with zero junctional epitopes for the Class I haplotype motifs considered here. Linkers of two, three, four, five, or six amino acids in length will produce no junctional epitopes.

To avoid junctional epitopes, the following amino acids MUST be avoided in a two amino acid linker between epitopes 5(N-terminal)-4(C-terminal). The linker is shown from left to right, with the most N-terminal residue of the linker occuring at the left, and the most C-terminal residue occuring to the right. Amino acids to be avoided are shown position by position in the linker.

| | | | |
|---|---|---|---|
| N-End | _{—} | _{—} | C-End |
| | L | P | |
| | M | R | |
| | P | E | |
| | R | V | |
| | E | I | |
| | V | L | |
| | K | K | |
| | Y | Y | |
| | I | | |

### Example 41

Junctional epitopes between epitopes 5 and 5 as a function of linker length. Epitope 5 at N-end, Epitope 5 at C-end

| | | | | | | | | | | | | | | | | | | | | | | | | | |
|---|---|---|---|---|---|---|---|---|---|---|---|---|---|---|---|---|---|---|---|---|---|---|---|---|---|
| Y | L | Q | L | V | F | G | I | E | V | _{—} | _{—} | _{—} | _{—} | _{—} | _{—} | Y | L | Q | L | V | F | G | I | E | V |
| | | | A₂ | | | | | A_{1c} | A₃ | | | | | | | A_{1c} | A₂ | | A₂ | A₂ | B₇ | | A₂ | | |
| | | | A₃ | A₃ | | | | B₄₄ | | | | | | | | A_{1b} | B₇ | | B₇ | B₇ | A₂₄ | | B₇ | | |
| | | | | | | | | | | | | | | | | B₄₄ | B₂₇ | | B₂₇ | | B₄₄ | | | | |
| | | | | | | | | | | | | | | | | | | | | | B₂₇ | | | | |

Linker Length in Amino Acids

| Class I MHC | 0 | 1 | 2 | 3 | 4 | 5 | 6 |
|---|---|---|---|---|---|---|---|
| A₂ | 1 | 0 | 0 | 2 | 0 | 0 | 0 |
| A_{1C} | 0 | 0 | 0 | 0 | 1 | 1 | 0 |
| B₄₄ | 1 | 1 | 0 | 0 | 0 | 1 | 1 |
| A₃ | 0 | 0 | 0 | 0 | 0 | 0 | 0 |
| Total | 2 | 1 | 0 | 2 | 1 | 2 | 1 |

The 5(N-terminal)-5(C-terminal) epitope pairing can be used in a polyepitope with zero junctional epitopes for the Class I haplotype motifs considered here. Linkers of two or three amino acids in length will produce no junctional epitopes.

To avoid junctional epitopes, the following amino acids MUST be avoided in a two amino acid linker between epitopes 5(N-terminal)-5(C-terminal). The linker is shown from left to right, with the most N-terminal residue of the linker occurring at the left, and the most C-terminal residue occurring to the right. Amino acids to be avoided are shown position by position in the linker.

| | | | |
|---|---|---|---|
| N-End | _{—} | _{—} | C-End |
| | L | P | |
| | | R | |
| | P | | |
| | R | V | |
| | E | I | |
| | V | L | |
| | K | K | |
| | Y | M | |
| | I | | |

### Example 42

Junctional epitopes between epitopes 5 and 6 as a function of linker length. Epitope 5 at N-end, Epitope 6 at C-end

| | | | | | | | | | | | | | | | | | | | | | | | | |
|---|---|---|---|---|---|---|---|---|---|---|---|---|---|---|---|---|---|---|---|---|---|---|---|---|
| Y | L | Q | L | V | F | G | I | E | V | _{—} | _{—} | _{—} | _{—} | _{—} | _{—} | I | M | I | G | V | L | V | G | V |
| | | | A₂ | | | | | A_{1c} | A₃ | | | | | | | A₂ | B₇ | A₂ | | A₂ | A₂ | A₂ | | |
| | | | A₃ | A₃ | | | | B₄₄ | | | | | | | | B₇ | | B₇ | | B₇ | B₇ | B₇ | | |
| | | | | | | | | | | | | | | | | | | | | | B₂₇ | | | |

Linker Length in Amino Acids

| Class I MHC | 0 | 1 | 2 | 3 | 4 | 5 | 6 |
|---|---|---|---|---|---|---|---|
| A₂ | 1 | 1 | 0 | 0 | 0 | 0 | 0 |
| A₃ | 0 | 0 | 0 | 0 | 0 | 0 | 0 |
| A_{1C} | 0 | 0 | 0 | 0 | 0 | 0 | 0 |
| B₄₄ | 0 | 0 | 0 | 0 | 0 | 0 | 0 |
| Total | 1 | 1 | 0 | 0 | 0 | 0 | 0 |

The 5(N-terminal)-6(C-terminal) epitope pairing can be used in a polyepitope with zero junctional epitopes for the Class I haplotype motifs considered here. Linkers of two, three, four, five, or six amino acids in length will produce no junctional epitopes.

To avoid junctional epitopes, the following amino acids MUST be avoided in a two amino acid linker between epitopes 5(N-terminal)-6(C-terminal). The linker is shown from left to right, with the most N-terminal residue of the linker occuring at the left, and the most C-terminal residue occuring to the right. Amino acids to be avoided are shown position by position in the linker.

| | | | |
|---|---|---|---|
| N-End | _ | _ | C-End |
| | L | L | |
| | | M | |
| | P | P | |
| | V | V | |
| | I | I | |
| | K | K | |
| | R | R | |
| | M | | |

### Example 43

Junctional epitopes between epitopes 5 and 7 as a function of linker length. Epitope 5 at N-end, Epitope 7 at C-end

| | | | | | | | | | | | | | | | | | | | | | | | | |
|---|---|---|---|---|---|---|---|---|---|---|---|---|---|---|---|---|---|---|---|---|---|---|---|---|
| Y | L | Q | L | V | F | G | I | E | V | _{—} | _{—} | _{—} | _{—} | _{—} | _{—} | Y | L | S | G | A | N | L | N | V |
| | | | A₂ | | | | | A_{1c} | | | | | | | | A_{1b} | A₂ | | | | | A₂ | | |
| | | | A₃ | A₃ | | | | B₄₄ | A₃ | | | | | | | A_{1c} | B₇ | | | | | B₇ | | |
| | | | | | | | | | | | | | | | | B₄₄ | B₂₇ | | | | | B₂₇ | | |

Linker Length in Amino Acids

| Class I MHC | 0 | 1 | 2 | 3 | 4 | 5 | 6 |
|---|---|---|---|---|---|---|---|
| A₂ | 1 | 0 | 0 | 0 | 0 | 0 | 0 |
| A₃ | 0 | 0 | 0 | 0 | 0 | 0 | 0 |
| A_{1C} | 0 | 0 | 0 | 0 | 1 | 1 | 0 |
| B₄₄ | 0 | 0 | 0 | 0 | 0 | 1 | 1 |
| Total | 1 | 0 | 0 | 0 | 1 | 2 | 1 |

The 5(N-terminal)-7(C-terminal) epitope pairing can be used in a polyepitope with zero junctional epitopes for the Class I haplotype motifs considered here. Linkers of one, two, or three amino acids in length will produce no junctional epitopes.

To avoid junctional epitopes, the following amino acids MUST be avoided in a one amino acid linker between epitopes 5(N-terminal)-7(C-terminal). The linker is shown from left to right, with the most N-terminal residue of the linker occuring at the left, and the most C-terminal residue occuring to the right. Amino acids to be avoided are shown position by position in the linker.

| | | | |
|---|---|---|---|
| N-End | _{—} | _{—} | C-End |
| | V | | |
| | I | | |
| | L | | |
| | K | | |
| | R | | |
| | M | | |
| | P | | |

### Example 44

Junctional epitopes between epitopes 5 and 8 as a function of linker length. Epitope 5 at N-end, Epitope 8 at C-end

| | | | | | | | | | | | | | | | | | | | | | | | | |
|---|---|---|---|---|---|---|---|---|---|---|---|---|---|---|---|---|---|---|---|---|---|---|---|---|
| Y | L | Q | L | V | F | G | I | E | V | _{—} | _{—} | _{—} | _{—} | _{—} | _{—} | R | L | L | Q | E | T | E | L | V |
| | | | A₂ | | | | | A_{1c} | A₃ | | | | | | | A₃ | A₂ | A₂ | | | | | A₂ | |
| | | | A₃ | A₃ | | | | B₄₄ | | | | | | | | B₂₇ | B₇ | B₇ | | | | | B₇ | |
| | | | | | | | | | | | | | | | | | B₂₇ | B₂₇ | | | | | B₂₇ | |

Linker Length in Amino Acids

| Class I MHC | 0 | 1 | 2 | 3 | 4 | 5 | 6 |
|---|---|---|---|---|---|---|---|
| A₂ | 1 | 0 | 0 | 0 | 0 | 0 | 0 |
| A₃ | 1 | 2 | 1 | 0 | 0 | 0 | 1 |
| A_{1C} | 0 | 0 | 0 | 0 | 0 | 0 | 0 |
| B₄₄ | 0 | 0 | 0 | 0 | 0 | 0 | 0 |
| Total | 2 | 2 | 1 | 0 | 0 | 0 | 1 |

The 5(N-terminal)-8(C-terminal) epitope pairing can be used in a polyepitope with zero junctional epitopes for the Class I haplotype motifs considered here. Linkers of three, four, or five amino acids in length will produce no junctional epitopes.

To avoid junctional epitopes, the following amino acids MUST be avoided in a three amino acid linker between epitopes 5(N-terminal)-8(C-terminal). The linker is shown from left to right, with the most N-terminal residue of the linker occuring at the left, and the most C-terminal residue occuring to the right. Amino acids to be avoided are shown position by position in the linker.

| | | | | |
|---|---|---|---|---|
| N-End | _ | _ | _ | C-End |
| | V | V | K | |
| | I | I | R | |
| | L | L | L | |
| | K | K | M | |
| | R | R | P | |

### Example 45

Junctional epitopes between epitopes 5 and 9 as a function of linker length. Epitope 5 at N end, Epitope 9 at C end

| | | | | | | | | | | | | | | | | | | | | | | | | |
|---|---|---|---|---|---|---|---|---|---|---|---|---|---|---|---|---|---|---|---|---|---|---|---|---|
| Y | L | Q | L | V | F | G | I | E | V | _{—} | _{—} | _{—} | _{—} | _{—} | _{—} | S | M | P | P | P | G | T | R | V |
| | | | A₂ | | | | | A_{1c} | | | A₃ | | | | | | B₇ | | | | | | B₂₇ | |
| | | | A₃ | A₃ | | | | B₄₄ | | | | | | | | | | | | | | | A₃ | |

Linker Length in Ammo Acids

| Class I MHC | 0 | 1 | 2 | 3 | 4 | 5 | 6 |
|---|---|---|---|---|---|---|---|
| A₂ | 0 | 0 | 0 | 0 | 0 | 0 | 0 |
| A₃ | 1 | 0 | 0 | 0 | 0 | 0 | 0 |
| A_{1C} | 0 | 0 | 0 | 0 | 0 | 0 | 0 |
| B₄₄ | 0 | 0 | 0 | 0 | 0 | 0 | 0 |
| Total | 1 | 0 | 0 | 0 | 0 | 0 | 0 |

The 5(N-terminal)-9(C-terminal) epitope pairing can be used in a polyepitope with zero junctional epitopes for the Class I haplotype motifs considered here. Linkers of one, two, three, four, five, or six amino acids in length will produce no junctional epitopes.

To avoid junctional epitopes, the following amino acids MUST be avoided in a one amino acid linker between epitopes 5(N-terminal)-9(C-terminal). The linker is shown from left to right, with the most N-terminal residue of the linker occurring at the left, and the most C-terminal residue occurring to the right. Amino acids to be avoided are shown position by position in the linker.

| | | |
|---|---|---|
| N End | _ | C End |
| | R | |
| | L | |
| | M | |
| | T | |
| | V | |
| | K | |
| | I | |

### Example 46

Junctional epitopes between epitopes 6 and 1 as a function of linker length. Epitope 6 at N end, Epitope 1 at C end

| | | | | | | | | | | | | | | | | | | | | | | | |
|---|---|---|---|---|---|---|---|---|---|---|---|---|---|---|---|---|---|---|---|---|---|---|---|
| I | M | I | G | V | L | V | G | V | _{—} | _{—} | _{—} | _{—} | _{—} | _{—} | K | L | C | P | V | Q | L | W | V |
| | A₂ | | | A₃ | A₂ | A₃ | | A₃ | | | | | | | A₃ | A₂ | | | A₂ | | A₂ | | |
| | A₃ | | | | A₃ | | | | | | | | | | B₂₇ | B₇ | | | B₇ | | B₇ | | |
| | | | | | | | | | | | | | | | | B₂₇ | | | | | B₂₇ | | |

Linker Length in Amino Acids

| Class I MHC | 0 | 1 | 2 | 3 | 4 | 5 | 6 |
|---|---|---|---|---|---|---|---|
| A₂ | 1 | 0 | 1 | 1 | 0 | 0 | 0 |
| A₃ | 1 | 0 | 1 | 2 | 2 | 1 | 1 |
| Total | 2 | 0 | 2 | 3 | 2 | 1 | 1 |

The 6(N-terminal)-1(C-terminal) epitope pairing can be used in a polyepitope with zero junctional epitopes for the Class I haplotype motifs considered here. Linkers of one amino acids in length will produce no junctional epitopes.

To avoid junctional epitopes, the following amino acids MUST be avoided in a one amino acid linker between epitopes 6(N-terminal)-1(C-terminal). The linker is shown from left to right, with the most N-terminal residue of the linker occuring at the left, and the most C-terminal residue occuring to the right. Amino acids to be avoided are shown position by position in the linker.

| | | |
|---|---|---|
| N End | _ | C End |
| | V | |
| | I | |
| | L | |
| | K | |
| | R | |
| | M | |
| | P | |

### Example 47

Junctional epitopes between epitopes 6 and 2 as a function of linker length. Epitope pair 42: Epitope 6 at N end, Epitope 2 at C end

| | | | | | | | | | | | | | | | | | | | | | | | | | | |
|---|---|---|---|---|---|---|---|---|---|---|---|---|---|---|---|---|---|---|---|---|---|---|---|---|---|---|
| I | M | I | G | V | L | V | G | V | _{—} | _{—} | _{—} | _{—} | _{—} | _{—} | A | K | F | V | A | A | W | T | L | K | A | AA |
| | A₂ | | | A₃ | A₂ | A₃ | | A₃ | | | | | | | | A₃ | B₇ | A₂ | | | | | | | | |
| | A₃ | | | | A₃ | | | | | | | | | | | B₂₇ | B₂₇ | B₇ | | | | | | | | |
| | | | | | | | | | | | | | | | | | A₂₄ | | | | | | | | | |
| | | | | | | | | | | | | | | | | | B₄₄ | | | | | | | | | |

Linker Length in Amino Acids

| Class I MHC | 0 | 1 | 2 | 3 | 4 | 5 | 6 |
|---|---|---|---|---|---|---|---|
| A₂ | 1 | 1 | 0 | 0 | 0 | 0 | 0 |
| A₃ | 0 | 1 | 2 | 2 | 1 | 1 | 1 |
| Total | 1 | 2 | 2 | 2 | 1 | 1 | 1 |

Allowing linkers of 0 to 6 amino acids between epitopes, the 6(N-terminal)-2 (C-terminal) epitope pairing cannot be used in a polyepitope with zero junctional epitopes for the Class I haplotype motifs considered here.

### Example 48

Junctional epitopes between epitopes 6 and 3 as a function of linker length. Epitope 6 at N-end, Epitope 3 at C-end

| | | | | | | | | | | | | | | | | | | | | | | | |
|---|---|---|---|---|---|---|---|---|---|---|---|---|---|---|---|---|---|---|---|---|---|---|---|
| I | M | I | G | V | L | V | G | V | _{—} | _{—} | _{—} | _{—} | _{—} | _{—} | K | V | A | E | L | V | H | F | L |
| | A₂ | | | A₃ | A₂ | A₃ | | A₃ | | | | | | | B₂₇ | A₂ | | | A₂ | A₂ | | B₇ | |
| | A₃ | | | | A₃ | | | | | | | | | | A₃ | B₇ | | | B₇ | B₇ | | B₂₇ | |
| | | | | | | | | | | | | | | | | | | | B₂₇ | | | A₂₄ | |
| | | | | | | | | | | | | | | | | | | | | | | B₄₄ | |

Linker Length in Amino Acids

| Class I MHC | 0 | 1 | 2 | 3 | 4 | 5 | 6 |
|---|---|---|---|---|---|---|---|
| A₂ | 1 | 0 | 1 | 1 | 0 | 0 | 0 |
| A₃ | 1 | 0 | 1 | 2 | 2 | 1 | 1 |
| Total | 2 | 0 | 2 | 3 | 2 | 1 | 1 |

The 6(N-terminal)-3(C-terminal) epitope pairing can be used in a polyepitope with zero junctional epitopes for the Class I haplotype motifs considered here. Linkers of one amino acids in length will produce no junctional epitopes.

To avoid junctional epitopes, the following amino acids MUST be avoided in a 1 amino acid linker between epitopes 6(N-terminal)-3(C-terminal). The linker is shown from left to right, with the most N-terminal residue of the linker occurring at the left, and the most C-terminal residue occurring to the right. Amino acids to be avoided are shown position by position in the linker.

| | | |
|---|---|---|
| N-End | _ | C-End |
| | V | |
| | I | |
| | L | |
| | K | |
| | R | |
| | P | |
| | Y | |
| | E | |

### Example 49

Junctional epitopes between epitopes 6 and 4 as a function of linker length. Epitope 6 at N-end, Epitope 4 at C-end

| | | | | | | | | | | | | | | | | | | | | | | | |
|---|---|---|---|---|---|---|---|---|---|---|---|---|---|---|---|---|---|---|---|---|---|---|---|
| I | M | I | G | V | L | V | G | V | _{—} | _{—} | _{—} | _{—} | _{—} | _{—} | V | V | L | G | V | V | F | G | I |
| | A₂ | | | A₃ | A₂ | A₃ | | A₃ | | | | | | | A₂ | A₂ | A₂ | | A₂ | A₂ | B₇ | | |
| | A₃ | | | | A₃ | | | | | | | | | | B₇ | B₇ | B₇ | | B₇ | B₇ | B₂₇ | | |
| | | | | | | | | | | | | | | | | | B₂₇ | | | | B₄₄ | | |
| | | | | | | | | | | | | | | | | | | | | | A₂₄ | | |

Linker Length in Amino Acids

| Class I MHC | 0 | 1 | 2 | 3 | 4 | 5 | 6 |
|---|---|---|---|---|---|---|---|
| A₂ | 2 | 1 | 2 | 2 | 1 | 0 | 0 |
| A₃ | 0 | 0 | 0 | 0 | 0 | 0 | 0 |
| Total | 2 | 1 | 2 | 2 | 1 | 0 | 0 |

The 6(N-terminal)-4(C-terminal) epitope pairing can be used in a polyepitope with zero junctional epitopes for the Class I haplotypes considered here. Linkers of five or six amino acids in length will produce no junctional epitopes.

To avoid junctional epitopes, the following amino acids MUST be avoided in a five amino acid linker between epitopes 6(N-terminal)-4(C-terminal). The linker is shown from left to right, with the most N-terminal residue of the linker occuring at the left, and the most C-terminal residue occuring to the right. Amino acids to be avoided are shown position by position in the linker.

| | | | | | | |
|---|---|---|---|---|---|---|
| N-End | _ | _ | _ | _ | _ | C-End |
| | L | L | L | L | P | |
| | M | M | M | M | R | |
| | P | P | P | P | E | |
| | R | | K | R | Y | |
| | V | | R | Y | V | |
| | I | | | K | I | |
| | K | | | V | L | |
| | | | | I | K | |
| | | | | E | | |

### Example 50

Junctional epitopes between epitopes 6 and 5 as a function of linker length Epitope 6 at N-end, Epitope 5 at C-end

| | | | | | | | | | | | | | | | | | | | | | | | | |
|---|---|---|---|---|---|---|---|---|---|---|---|---|---|---|---|---|---|---|---|---|---|---|---|---|
| I | M | I | G | V | L | V | G | V | _{—} | _{—} | _{—} | _{—} | _{—} | _{—} | Y | L | Q | L | V | F | G | I | E | V |
| | A₂ | | | A₃ | A₂ | A₃ | | A₃ | | | | | | | A_{1b} | A₂ | | A₂ | A₂ | A₂₄ | | A₂ | | |
| | A₃ | | | | A₃ | | | | | | | | | | A_{1c} | B₇ | | B₇ | B₇ | B₇ | | B₇ | | |
| | | | | | | | | | | | | | | | B₄₄ | B₂₇ | | B₂₇ | | B₄₄ | | | | |
| | | | | | | | | | | | | | | | | | | | | B₂₇ | | | | |

Linker Length in Acids Amino

| Class I MHC | 0 | 1 | 2 | 3 | 4 | 5 | 6 |
|---|---|---|---|---|---|---|---|
| A₂ | 2 | 1 | 1 | 1 | 0 | 0 | 0 |
| A₃ | 0 | 0 | 0 | 0 | 0 | 0 | 0 |
| Total | 2 | 1 | 1 | 1 | 0 | 0 | 0 |

The 6(N-terminal)-5(C-terminal) epitope pairing can be used in a polyepitope with zero junctional epitopes for the Class I haplotype motifs considered here. Linkers of four, five, or six amino acids in length will produce no junctional epitopes.

To avoid junctional epitopes, the following amino acids MUST be avoided in a four amino acid linker between epitopes 6(N-terminal)-5(C-terminal). The linker is shown from left to right, with the most N-terminal residue of the linker occuring at the left, and the most C-terminal residue occuring to the right. Amino acids to be avoided are shown position by position in the linker.

| | | | | | |
|---|---|---|---|---|---|
| N-End | _ | _ | _ | _ | C-End |
| | L | L | P | L | |
| | M | M | Y | M | |
| | P | P | E | P | |
| | R | | K | I | |
| | V | Y | R | K | |
| | I | E | | R | |
| | K | R | | V | |

### Example 51

Junctional epitopes between epitopes 6 and 6 as a function of linker length

Epitope 6 N end, Epitope 6 C end

| | | | | | | | | | | | | | | | | | | | | | | | |
|---|---|---|---|---|---|---|---|---|---|---|---|---|---|---|---|---|---|---|---|---|---|---|---|
| I | M | I | G | V | L | V | G | V | _{—} | _{—} | _{—} | _{—} | _{—} | _{—} | I | M | I | G | V | L | V | G | V |
| | A₂ | | | A₃ | A₂ | A₃ | | A₃ | | | | | | | A₂ | B₇ | A₂ | | A₂ | A₂ | A₂ | | |
| | A₃ | | | | A₃ | | | | | | | | | | B₇ | | B₇ | | B₇ | B₇ | B₇ | | |
| | | | | | | | | | | | | | | | | | | | | B₂₇ | | | |

Linker Length in Amino Acids

| Class I MHC | 0 | 1 | 2 | 3 | 4 | 5 | 6 |
|---|---|---|---|---|---|---|---|
| A₂ | 2 | 1 | 1 | 1 | 1 | 0 | 0 |
| A₃ | 0 | 0 | 0 | 0 | 0 | 0 | 0 |
| Total | 2 | 1 | 1 | 1 | 1 | 0 | 0 |

The 6(N-terminal)-6(C-terminal) epitope pairing can be used in a polyepitope with zero junctional epitopes for the Class I haplotype motifs considered here. Linkers of five or six amino acids in length will produce no junctional epitopes.

To avoid junctional epitopes, the following amino acids MUST be avoided in a five amino acid linker between epitopes 6(N-terminal)-6(C-terminal). The linker is shown from left to right, with the most N-terminal residue of the linker occuring at the left, and the most C-terminal residue occuring to the right. Amino acids to be avoided are shown position by position in the linker.

| | | | | | | |
|---|---|---|---|---|---|---|
| N-End | _ | _ | _ | _ | _ | C-End |
| | V | L | K | V | V | |
| | I | M | R | I | I | |
| | L | P | L | L | L | |
| | K | | M | K | K | |
| | R | | P | R | R | |
| | M | | | M | M | |
| | P | | | P | P | |

### Example 52

Junctional epitopes between epitopes 6 and 7 as a function of linker length Epitope 6 at N-end, Epitope 7 at C-end

| | | | | | | | | | | | | | | | | | | | | | | | |
|---|---|---|---|---|---|---|---|---|---|---|---|---|---|---|---|---|---|---|---|---|---|---|---|
| I | M | I | G | V | L | V | G | V | _{—} | _{—} | _{—} | _{—} | _{—} | _{—} | Y | L | S | G | A | N | L | N | V |
| | A₂ | | | A₃ | A₂ | A₃ | | A₃ | | | | | | | A_{1b} | A₂ | | | | | A₂ | | |
| | A₃ | | | | A₃ | | | | | | | | | | A_{1c} | B₇ | | | | | B₇ | | |
| | | | | | | | | | | | | | | | B₄₄ | B₂₇ | | | | | B₂₇ | | |

Linker Length in Amino Acids

| Class 1 MHC | 0 | 1 | 2 | 3 | 4 | 5 | 6 |
|---|---|---|---|---|---|---|---|
| A₂ | 0 | 0 | 1 | 1 | 0 | 0 | 0 |
| A₃ | 0 | 0 | 0 | 0 | 0 | 0 | 0 |
| Total | 0 | 0 | 1 | 1 | 0 | 0 | 0 |

The 6(N-terminal)-7(C-terminal) epitope pairing can be used in a polyepitope with zero, one, four, five, or six junctional epitopes for the Class I haplotype motifs considered here. Linkers of zero amino acids in length will produce no, junctional epitopes. As there are no amino acids in a zero amino acid linker, there are no constraints on the amino acids used in a zero length linker.

### Example 53

Junctional epitopes between epitopes 6 and 8 as a function of linker length Epitope 6 at N-end, Epitope 8 at C-end

| | | | | | | | | | | | | | | | | | | | | | | | |
|---|---|---|---|---|---|---|---|---|---|---|---|---|---|---|---|---|---|---|---|---|---|---|---|
| I | M | I | G | V | L | V | G | V | _{—} | _{—} | _{—} | _{—} | _{—} | _{—} | R | L | L | Q | E | T | E | L | V |
| | A₂ | | | A₃ | A₂ | A₃ | | A₃ | | | | | | | A₃ | A₂ | A₂ | | | | | A₂ | |
| | A₃ | | | | A₃ | | | | | | | | | | B₂₇ | B₇ | B₇ | | | | | B₇ | |
| | | | | | | | | | | | | | | | | B₂₇ | B₂₇ | | | | | B₂₇ | |

Linker Length in Amino Acids

| Class I MHC | 0 | 1 | 2 | 3 | 4 | 5 | 6 |
|---|---|---|---|---|---|---|---|
| A₂ | 0 | 1 | 2 | 1 | 0 | 0 | 0 |
| A₃ | 1 | 0 | 1 | 2 | 2 | 1 | 1 |
| Total | 1 | 1 | 3 | 3 | 2 | 1 | 1 |

Allowing linkers of 0 to 6 amino acids between epitopes, the 6(N-terminal)-8 (C-terminal) epitope pairing cannot be used in a polyepitope with zero junctional epitopes for the Class I haplotype motifs considered here.

### Example 54

Junctional epitopes between epitopes 6 and 9 as a function of linker length Epitope 6 at N-end, Epitope 9 at C-end

| | | | | | | | | | | | | | | | | | | | | | | | |
|---|---|---|---|---|---|---|---|---|---|---|---|---|---|---|---|---|---|---|---|---|---|---|---|
| I | M | I | G | V | L | V | G | V | _{—} | _{—} | _{—} | _{—} | _{—} | _{—} | S | M | P | P | P | G | T | R | V |
| | A₂ | | | A₃ | A₂ | A₃ | | A₃ | | | | | | | | B₇ | | | | | | B₂₇ | |
| | A₃ | | | | A₃ | | | | | | | | | | | | | | | | | A₃ | |

Linker Length in Amino Acids

| Class I MHC | 0 | 1 | 2 | 3 | 4 | 5 | 6 |
|---|---|---|---|---|---|---|---|
| A₂ | 0 | 0 | 0 | 0 | 0 | 0 | 0 |
| A₃ | 1 | 0 | 0 | 0 | 0 | 0 | 0 |
| Total | 1 | 0 | 0 | 0 | 0 | 0 | 0 |

The 6(N-terminal)-9(C-terminal) epitope pairing can be used in a polyepitope with zero junctional epitopes for the Class I haplotype motifs considered here. Linkers of one, two, three, four, five, or six amino acids in length will produce no junctional epitopes.

To avoid junctional epitopes, the following amino acids MUST be avoided in a one amino acid linker between epitopes 6(N-terminal)-9(C-terminal). The linker is shown from left to right, with the most N-terminal residue of the linker occuring at the left, and the most C-terminal residue occuring to the right. Amino acids to be avoided are shown position by position in the linker.

| | | |
|---|---|---|
| N-End | _ | C-End |
| | R | |
| | L | |
| | M | |
| | T | |
| | V | |
| | I | |
| | K | |

### Example 55

Junctional epitopes between epitopes 7 and 1 as a function of linker length Epitope 7 at N-end, Epitope 1 at C-end

| | | | | | | | | | | | | | | | | | | | | | | | |
|---|---|---|---|---|---|---|---|---|---|---|---|---|---|---|---|---|---|---|---|---|---|---|---|
| Y | L | S | G | A | N | L | N | V | _{—} | _{—} | _{—} | _{—} | _{—} | _{—} | K | L | C | P | V | Q | L | W | V |
| | A₂ | A_{1b} | | | | A₂ | | A₃ | | | | | | | A₃ | A₂ | | | A₂ | | A₂ | | |
| | A₃ | | | | | A₃ | | | | | | | | | B₂₇ | B₇ | | | B₇ | | B₇ | | |
| | | | | | | | | | | | | | | | | B₂₇ | | | | | B₂₇ | | |

Linker Length in Amino Acids

| Class I MHC | 0 | 1 | 2 | 3 | 4 | 5 | 6 |
|---|---|---|---|---|---|---|---|
| A₂ | 1 | 1 | 0 | 1 | 1 | 0 | 0 |
| A_{1b} | 0 | 0 | 0 | 0 | 0 | 0 | 0 |
| A₃ | 1 | 0 | 0 | 0 | 1 | 1 | 1 |
| Total | 2 | 1 | 0 | 1 | 2 | 1 | 1 |

The 7(N-terminal)-1(C-terminal) epitope pairing can be used in a polyepitope with zero junctional epitopes for the Class I haplotype motifs considered here. Linkers of two amino acids in length will produce no junctional epitopes.

To avoid junctional epitopes, the following amino acids MUST be avoided in a two amino acid linker between epitopes 7(N-terminal)-1(C-terminal). The linker is shown from left to right, with the most N-terminal residue of the linker occuring at the left, and the most C-terminal residue occuring to the right. Amino acids to be avoided are shown position by position in the linker.

| | | | |
|---|---|---|---|
| N-End | _ | _ | C-End |
| | V | Y | |
| | I | L | |
| | L | M | |
| | K | P | |
| | R | R | |
| | Y | | |
| | M | | |
| | P | | |

### Example 56

Junctional epitopes between epitopes 7 and 2 as a function of linker length Epitope 7 at N-end, Epitope 2 at C-end

| | | | | | | | | | | | | | | | | | | | | | | | | | | |
|---|---|---|---|---|---|---|---|---|---|---|---|---|---|---|---|---|---|---|---|---|---|---|---|---|---|---|
| Y | L | S | G | A | N | L | N | V | _{—} | _{—} | _{—} | _{—} | _{—} | _{—} | A | K | F | V | A | A | W | T | L | K | A | AA |
| | A₂ | A_{1b} | | | | A₂ | | A₃ | | | | | | | | A₃ | B₇ | A₂ | | | | | | | | |
| | A₃ | | | | | A₃ | | | | | | | | | | B₂₇ | B₂₇ | B₇ | | | | | | | | |
| | | | | | | | | | | | | | | | | | A₂₄ | | | | | | | | | |
| | | | | | | | | | | | | | | | | | B₄₄ | | | | | | | | | |

Linker Length in Amino Acids

| Class I MHC | 0 | 1 | 2 | 3 | 4 | 5 | 6 |
|---|---|---|---|---|---|---|---|
| A₂ | 0 | 1 | 1 | 0 | 0 | 0 | 0 |
| A₃ | 0 | 0 | 0 | 1 | 1 | 1 | 1 |
| A_{IC} | 0 | 0 | 0 | 0 | 0 | 0 | 0 |
| Total | 0 | 1 | 1 | 1 | 1 | 1 | 1 |

The 7(N-terminal)-2(C-terminal) epitope pairing can be used in a polyepitope with zero junctional epitopes for the Class I haplotype motifs considered here. Linkers of zero amino acids in length will produce no junctional epitopes. As there are no amino acids in a zero amino acid linker, there are no constraints on the amino acids used in a zero length linker.

### Example 57

Junctional epitopes between epitopes 7 and 3 as a function of linker length Epitope 7 at N-end, Epitope 3 at C-end

| | | | | | | | | | | | | | | | | | | | | | | | |
|---|---|---|---|---|---|---|---|---|---|---|---|---|---|---|---|---|---|---|---|---|---|---|---|
| Y | L | S | G | A | N | L | N | V | _{—} | _{—} | _{—} | _{—} | _{—} | _{—} | K | V | A | E | L | V | H | F | L |
| | A₂ | A_{1b} | | | | A₂ | | A₃ | | | | | | | B₂₇ | A₂ | | | A₂ | A₂ | | B₇ | |
| | A₃ | | | | | A₃ | | | | | | | | | A₃ | B₇ | | | B₇ | B₇ | | B₂₇ | |
| | | | | | | | | | | | | | | | | | | | B₂₇ | | | A₂₄ | |
| | | | | | | | | | | | | | | | | | | | | | | B₄₄ | |

Linker Length in Amino Acids

| Class I MHC | 0 | 1 | 2 | 3 | 4 | 5 | 6 |
|---|---|---|---|---|---|---|---|
| A₂ | 2 | 1 | 0 | 1 | 1 | 0 | 0 |
| A₃ | 1 | 0 | 0 | 0 | 1 | 1 | 1 |
| A_{1b} | 0 | 0 | 0 | 0 | 0 | 0 | 0 |
| Total | 3 | 1 | 0 | 1 | 2 | 1 | 1 |

The 7(N-terminal)-3(C-terminal) epitope pairing can be used in a polyepitope with zero junctional epitopes for the Class I haplotype motifs considered here. Linkers of two amino acids in length will produce no junctional epitopes.

To avoid junctional epitopes, the following amino acids MUST be avoided in a two amino acid linker between epitopes 7(N-terminal)-3(C-terminal). The linker is shown from left to right, with the most N-terminal residue of the linker occuring at the left, and the most C-terminal residue occuring to the right. Amino acids to be avoided are shown position by position in the linker.

| | | | |
|---|---|---|---|
| N-End | _ | _ | C-End |
| | L | P | |
| | M | R | |
| | P | Y | |
| | V | E | |
| | I | | |
| | K | | |
| | R | | |
| | Y | | |

### Example 58

Junctional epitopes between epitopes 7 and 4 as a function of linker length Epitope 7 at N end, Epitope 4 at C end

| | | | | | | | | | | | | | | | | | | | | | | | |
|---|---|---|---|---|---|---|---|---|---|---|---|---|---|---|---|---|---|---|---|---|---|---|---|
| Y | L | S | G | A | N | L | N | V | _{—} | _{—} | _{—} | _{—} | _{—} | _{—} | V | V | L | G | V | V | F | G | I |
| | A₂ | A_{1b} | | | | A₂ | | A₃ | | | | | | | A₂ | A₂ | A₂ | | A₂ | A₂ | B₇ | | |
| | A₃ | | | | | A₃ | | | | | | | | | B₇ | B₇ | B₇ | | B₇ | B₇ | B₂₇ | | |
| | | | | | | | | | | | | | | | | | B₂₇ | | | | B₄₄ | | |
| | | | | | | | | | | | | | | | | | | | | | A₂₄ | | |

Linker Length in Amino Acids

| Class 1 MHC | 0 | 1 | 2 | 3 | 4 | 5 | 6 |
|---|---|---|---|---|---|---|---|
| A₂ | 3 | 1 | 1 | 2 | 2 | 1 | 0 |
| A₃ | 0 | 0 | 0 | 0 | 0 | 0 | 0 |
| A_{1b} | 0 | 0 | 0 | 0 | 0 | 0 | 0 |
| Total | 3 | 1 | 1 | 2 | 2 | 1 | 0 |

The 7(N-terminal)-4(C-terminal) epitope pairing can be used in a polyepitope with zero junctional epitopes for the Class I haplotype motifs considered here. Linkers of six amino acids in length will produce no junctional epitopes.

To avoid junctional epitopes, the following amino acids MUST be avoided in a six amino acid linker between epitopes 7(N-terminal)-4(C-terminal). The linker is shown from left to right, with the most N-terminal residue of the linker occuring at the left, and the most C-terminal residue occuring to the right. Amino acids to be avoided are shown position by position in the linker.

| | | | | | | | |
|---|---|---|---|---|---|---|---|
| N-End | _ | _ | _ | _ | _ | _ | C-End |
| | L | L | L | L | L | P | |
| | M | M | M | M | M | R | |
| | P | P | P | P | P | E | |
| | R | R | | | R | Y | |
| | V | Y | | | E | V | |
| | I | | | | Y | I | |
| | K | | | | V | L | |
| | Y | | | | I | K | |
| | | | | | K | M | |

### Example 59

Junctional epitopes between epitopes 7 and 5 as a function of linker length Epitope 7 at N end, Epitope 5 at C end

| | | | | | | | | | | | | | | | | | | | | | | | | |
|---|---|---|---|---|---|---|---|---|---|---|---|---|---|---|---|---|---|---|---|---|---|---|---|---|
| Y | L | S | G | A | N | L | N | V | _{—} | _{—} | _{—} | _{—} | _{—} | _{—} | Y | L | Q | L | V | F | G | I | E | V |
| | A₂ | A_{1b} | | | | A₂ | | A₃ | | | | | | | A_{1b} | A₂ | | A₂ | A₂ | A₂₄ | | | | |
| | A₃ | | | | | A₃ | | | | | | | | | A_{1c} | B₇ | | B₇ | B₇ | B₇ | | | | |
| | | | | | | | | | | | | | | | B₄₄ | B₂₇ | | B₂₇ | | B₄₄ | | | | |
| | | | | | | | | | | | | | | | | | | | | B₂₇ | | | | |

Linker Length in Amino Acids

| Class I MHC | 0 | 1 | 2 | 3 | 4 | 5 | 6 |
|---|---|---|---|---|---|---|---|
| A₂ | 1 | 2 | 1 | 1 | 1 | 0 | 0 |
| A₃ | 0 | 0 | 0 | 0 | 0 | 0 | 0 |
| A_{1b} | 1 | 1 | 0 | 0 | 0 | 0 | 0 |
| Total | 2 | 3 | 1 | 1 | 1 | 0 | 0 |

The 7(N-terminal)-5(C-terminal) epitope pairing can be used in a polyepitope with zero junctional epitopes for the Class I haplotype motifs considered here. Linkers of five or six amino acids in length will produce no junctional epitopes.

To avoid junctional epitopes, the following amino acids MUST be avoided in a five amino acid linker between epitopes 7(N-terminal)-5(C-terminal). The linker is shown from left to right, with the most N-terminal residue of the linker occuring at the left, and the most C-terminal residue occuring to the right. Amino acids to be avoided are shown position by position in the linker.

| | | | | | | |
|---|---|---|---|---|---|---|
| N-End | _{—} | _{—} | _{—} | _{—} | _{—} | C-End |
| | L | L | L | P | L | |
| | M | M | M | Y | M | |
| | P | P | P | E | P | |
| | | R | Y | R | V | |
| | V | Y | R | | I | |
| | I | | E | | K | |
| | K | | | | R | |
| | R | | | | | |
| | Y | | | | | |

### Example 60

Junctional epitopes between epitopes 7 and 6 as a function of linker length Epitope 7 at N end, Epitope 6 at C end

| | | | | | | | | | | | | | | | | | | | | | | | |
|---|---|---|---|---|---|---|---|---|---|---|---|---|---|---|---|---|---|---|---|---|---|---|---|
| Y | L | S | G | A | N | L | N | V | _{—} | _{—} | _{—} | _{—} | _{—} | _{—} | I | M | I | G | V | L | V | G | V |
| | A₂ | A_{1b} | | | | A₂ | | A₃ | | | | | | | A₂ | B₇ | A₂ | | A₂ | A₂ | A₂ | | |
| | A₃ | | | | | A₃ | | | | | | | | | B₇ | | B₇ | | B₇ | B₇ | B₇ | | |
| | | | | | | | | | | | | | | | | | | | | B₂₇ | | | |

Linker Length in Acids Amino

| Class I MHC | 0 | 1 | 2 | 3 | 4 | 5 | 6 |
|---|---|---|---|---|---|---|---|
| A₂ | 3 | 1 | 1 | 1 | 1 | 1 | 0 |
| A₃ | 0 | 0 | 0 | 0 | 0 | 0 | 0 |
| A_{IC} | 0 | 0 | 0 | 0 | 0 | 0 | 0 |
| Total | 3 | 1 | 1 | 1 | 1 | 1 | 0 |

The 7(N-terminal)-6(C-terminal) epitope pairing can be used in a polyepitope with zero junctional epitopes for the Class I haplotype motifs considered here. Linkers of six amino acids in length will produce no junctional epitopes.

To avoid junctional epitopes, the following amino acids MUST be avoided in a six amino acid linker between epitopes 7(N-terminal)-6(C-terminal). The linker is shown from left to right, with the most N-terminal residue of the linker occuring at the left, and the most C-terminal residue occuring to the right. Amino acids to be avoided are shown position by position in the linker.

| | | | | | | | |
|---|---|---|---|---|---|---|---|
| N-End | _{—} | _{—} | _{—} | _{—} | _{—} | _{—} | C-End |
| | L | L | L | L | L | L | |
| | M | M | M | M | M | M | |
| | P | P | P | P | P | P | |
| | I | Y | | R | R | V | |
| | V | | | | V | I | |
| | K | | | | I | K | |
| | R | | | | K | R | |
| | Y | | | | | | |

### Example 61

Junctional epitopes between epitopes 7 and 7 as a function of linker length Epitope 7 at N-end, Epitope 7 at C-end

| | | | | | | | | | | | | | | | | | | | | | | | |
|---|---|---|---|---|---|---|---|---|---|---|---|---|---|---|---|---|---|---|---|---|---|---|---|
| Y | L | S | G | A | N | L | N | V | _{—} | _{—} | _{—} | _{—} | _{—} | _{—} | Y | L | S | G | A | N | L | N | V |
| | A₂ | A_{1b} | | | | A₂ | | A₃ | | | | | | | A_{1b} | A₂ | | | | | A₂ | | |
| | A₃ | | | | | A₃ | | | | | | | | | A_{1c} | B₇ | | | | | B₇ | | |
| | | | | | | | | | | | | | | | B₄₄ | B₂₇ | | | | | B₂₇ | | |

Linker Length in Amino Acids

| Class I MHC | 0 | 1 | 2 | 3 | 4 | 5 | 6 |
|---|---|---|---|---|---|---|---|
| A₂ | 0 | 0 | 0 | 1 | 1 | 0 | 0 |
| A₃ | 0 | 0 | 0 | 0 | 0 | 0 | 0 |
| A_{1b} | 1 | 1 | 0 | 0 | 0 | 0 | 0 |
| Total | 1 | 1 | 0 | 1 | 1 | 0 | 0 |

The 7(N-terminal)-7(C-terminal) epitope pairing can be used in a polyepitope with zero junctional epitopes for the Class I haplotype motifs considered here. Linkers of two, five, or six amino acids in length will produce no junctional epitopes.

To avoid junctional epitopes, the following amino acids MUST be avoided in a two amino acid linker between epitopes 7(N-terminal)-7(C-terminal). The linker is shown from left to right, with the most N-terminal residue of the linker occuring at the left, and the most C-terminal residue occuring to the right. Amino acids to be avoided are shown position by position in the linker.

| | | | |
|---|---|---|---|
| N-End | _{—} | _{—} | C-End |
| | L | L | |
| | M | M | |
| | P | P | |
| | I | Y | |
| | V | R | |
| | K | | |
| | R | | |
| | Y | | |

### Example 62

Junctional epitopes between epitopes 7 and 8 as a function of linker length Epitope 7 at N-end, Epitope 8 at C-end

| | | | | | | | | | | | | | | | | | | | | | | | |
|---|---|---|---|---|---|---|---|---|---|---|---|---|---|---|---|---|---|---|---|---|---|---|---|
| Y | L | S | G | A | N | L | N | V | _{—} | _{—} | _{—} | _{—} | _{—} | _{—} | R | L | L | Q | E | T | E | L | V |
| | A₂ | A_{1b} | | | | | A₂ | A₃ | | | | | | | A₃ | A₂ | A₂ | | | | | A₂ | |
| | A₃ | | | | | | A₃ | | | | | | | | B₂₇ | B₇ | B₇ | | | | | B₇ | |
| | | | | | | | | | | | | | | | | B₂₇ | B₂₇ | | | | | B₂₇ | |

Linker Length in Amino Acids

| Class I MHC | 0 | 1 | 2 | 3 | 4 | 5 | 6 |
|---|---|---|---|---|---|---|---|
| A₂ | 0 | 0 | 1 | 2 | 1 | 0 | 0 |
| A₃ | 1 | 0 | 0 | 0 | 1 | 1 | 1 |
| A_{1b} | 0 | 0 | 0 | 0 | 0 | 0 | 0 |
| Total | 1 | 0 | 1 | 2 | 2 | 1 | 1 |

The 7(N-terminal)-8(C-terminal) epitope pairing can be used in a polyepitope with zero junctional epitopes for the Class I haplotype motifs considered here. Linkers of one amino acids in length will produce no junctional epitopes.

To avoid junctional epitopes, the following amino acids MUST be avoided in a one amino acid linker between epitopes 7(N-terminal)-8(C-terminal). The linker is shown from left to right, with the most N-terminal residue of the linker occuring at the left, and the most C-terminal residue occuring to the right. Amino acids to be avoided are shown position by position in the linker.

| | | |
|---|---|---|
| N-End | _{—} | C-End |
| | L | |
| | M | |
| | P | |
| | R | |
| | V | |
| | I | |
| | K | |
| | Y | |

### Example 63

Junctional epitopes between epitopes 7 and 9 as a function of linker length Epitope 7 at N-end, Epitope 9 at C-end

| | | | | | | | | | | | | | | | | | | | | | | | |
|---|---|---|---|---|---|---|---|---|---|---|---|---|---|---|---|---|---|---|---|---|---|---|---|
| Y | L | S | G | A | N | L | N | V | _{—} | _{—} | _{—} | _{—} | _{—} | _{—} | S | M | P | P | P | G | T | R | V |
| | A₂ | A_{1b} | | | | A₂ | | A₃ | | | | | | | | B₇ | | | | | | B₂₇ | |
| | A₃ | | | | | A₃ | | | | | | | | | | | | | | | | A₃ | |

Linker Length in Amino Acids

| Class I MHC | 0 | 1 | 2 | 3 | 4 | 5 | 6 |
|---|---|---|---|---|---|---|---|
| A₂ | 0 | 0 | 0 | 0 | 0 | 0 | 0 |
| A₃ | 1 | 0 | 0 | 0 | 0 | 0 | 0 |
| A_{1b} | 0 | 0 | 0 | 0 | 0 | 0 | 0 |
| Total | 1 | 0 | 0 | 0 | 0 | 0 | 0 |

The 7(N-terminal)-9(C-terminal) epitope pairing can be used in a polyepitope with zero junctional epitopes for the Class I haplotypes considered here. Linkers of one, two, three, four, five, or six amino acids in length will produce no junctional epitopes.

To avoid junctional epitopes, the following amino acids MUST be avoided in a one amino acid linker between epitopes 7(N-terminal)-9(C-terminal). The linker is shown from left to right, with the most N-terminal residue of the linker occuring at the left, and the most C-terminal residue occuring to the right. Amino acids to be avoided are shown position by position in the linker.

| | | |
|---|---|---|
| N-End | _{—} | C-End |
| | V | |
| | I | |
| | L | |
| | K | |
| | R | |
| | Y | |
| | M | |
| | T | |

### Example 64

Junctional epitopes between epitopes 8 and 1 as a function of linker length Epitope 8 at N-end, Epitope 1 at C-end

| | | | | | | | | | | | | | | | | | | | | | | | |
|---|---|---|---|---|---|---|---|---|---|---|---|---|---|---|---|---|---|---|---|---|---|---|---|
| R | L | L | Q | E | T | E | L | V | _{—} | _{—} | _{—} | _{—} | _{—} | _{—} | K | L | C | P | V | Q | L | W | V |
| | A₂ | A₂ | | A_{1c} | A_{1b} | A_{1c} | A₂ | A₃ | | | | | | | A₃ | A₂ | | | A₂ | | A₂ | | |
| | A₃ | A₃ | | B₄₄ | A₃ | B₄₄ | A₃ | | | | | | | | B₂₇ | B₇ | | | B₇ | | B₇ | | |
| | | | | | | | | | | | | | | | | B₂₇ | | | | | B₂₇ | | |

Linker Length in Amino Acids

| Class I MHC | 0 | 1 | 2 | 3 | 4 | 5 | 6 |
|---|---|---|---|---|---|---|---|
| A₂ | 2 | 1 | 1 | 0 | 1 | 1 | 0 |
| A₃ | 2 | 1 | 0 | 1 | 1 | 1 | 2 |
| A_{1C} | 0 | 0 | 0 | 0 | 0 | 0 | 0 |
| B₄₄ | 0 | 0 | 0 | 0 | 0 | 0 | 0 |
| A_{1b} | 0 | 0 | 0 | 0 | 0 | 0 | 0 |
| Total | 4 | 2 | 1 | 1 | 2 | 2 | 2 |

Allowing linkers of 0 to 6 amino acids between epitopes, the 8(N-terminal)-1 (C-terminal) epitope pairing cannot be used in a polyepitope with zero junctional epitopes for the Class I haplotype motifs considered here.

### Example 65

Junctional epitopes between epitopes 8 and 2 as a function of linker length Epitope 8 at N-end, Epitope 2 at C-end

| | | | | | | | | | | | | | | | | | | | | | | | | |
|---|---|---|---|---|---|---|---|---|---|---|---|---|---|---|---|---|---|---|---|---|---|---|---|---|
| R | L | L | Q | E | T | E | L | V | _{—} | _{—} | _{—} | _{—} | _{—} | _{—} | A | K | F | V | AA | W | T | L | K | AAA |
| | A₂ | A₂ | | A_{1c} | A_{1b} | A_{1c} | A₂ | A₃ | | | | | | | | | A₃ | B₇ | A₂ | | | | | |
| | A₃ | A₃ | | B₄₄ | A₃ | B₄₄ | A₃ | | | | | | | | | B₂₇ | B₂₇ | B₇ | | | | | | |
| | | | | | | | | | | | | | | | | | A₂₄ | | | | | | | |
| | | | | | | | | | | | | | | | | | B₄₄ | | | | | | | |

Linker Length in Amino Acids

| Class I MHC | 0 | 1 | 2 | 3 | 4 | 5 | 6 |
|---|---|---|---|---|---|---|---|
| A₂ | 0 | 0 | 1 | 1 | 0 | 0 | 0 |
| A₃ | 1 | 0 | 1 | 1 | 1 | 2 | 1 |
| A_{1C} | 0 | 0 | 0 | 0 | 0 | 0 | 0 |
| A_{1b} | 0 | 0 | 0 | 0 | 0 | 0 | 0 |
| B₄₄ | 1 | 1 | 1 | 1 | 0 | 0 | 0 |
| Total | 2 | 1 | 3 | 3 | 1 | 2 | 1 |

Allowing linkers of 0 to 6 amino acids between epitopes, the 8(N-terminal)-2(C-terminal) epitope pairing cannot be used in a polyepitope with zero junctional epitopes for the Class I haplotype motifs considered here.

### Example 66

Junctional epitopes between epitopes 8 and 3 as a function of linker length Epitope 8 at N-end, Epitope 3 at C-end

| | | | | | | | | | | | | | | | | | | | | | | | |
|---|---|---|---|---|---|---|---|---|---|---|---|---|---|---|---|---|---|---|---|---|---|---|---|
| R | L | L | Q | E | T | E | L | V | _{—} | _{—} | _{—} | _{—} | _{—} | _{—} | K | V | A | E | L | V | H | F | L |
| | A₂ | A₂ | | A_{1c} | A₃ | A_{1c} | A₂ | A₃ | | | | | | | B₂₇ | A₂ | | | A₂ | A₂ | | B₇ | |
| | A₃ | A₃ | | B₄₄ | A_{1b} | B₄₄ | A₃ | | | | | | | | A₃ | B₇ | | | B₇ | B₇ | | B₂₇ | |
| | | | | | | | | | | | | | | | | | | | B₂₇ | | | A₂₄ | |
| | | | | | | | | | | | | | | | | | | | | | | B₄₄ | |

Linker Length in Amino Acids

| Class I MHC | 0 | 1 | 2 | 3 | 4 | 5 | 6 |
|---|---|---|---|---|---|---|---|
| A₂ | 2 | 2 | 1 | 0 | 1 | 1 | 1 |
| A₃ | 2 | 1 | 0 | 1 | 1 | 1 | 2 |
| A_{1C} | 0 | 0 | 0 | 0 | 0 | 0 | 0 |
| A_{1b} | 0 | 0 | 0 | 0 | 0 | 0 | 0 |
| B₄₄ | 0 | 0 | 0 | 0 | 0 | 0 | 0 |
| Total | 4 | 3 | 1 | 1 | 2 | 2 | 3 |

Allowing linkers of 0 to 6 amino acids between epitopes, the 8(N-terminal)-3 (C-terminal) epitope pairing cannot be used in a polyepitope with zero junctional epitopes for the Class I haplotype motifs considered here.

### Example 67

Junctional epitopes between epitopes 8 and 4 as a function of linker length Epitope 8 at N-end, Epitope 4 at C-end

| | | | | | | | | | | | | | | | | | | | | | | | |
|---|---|---|---|---|---|---|---|---|---|---|---|---|---|---|---|---|---|---|---|---|---|---|---|
| R | L | L | Q | E | T | E | L | V | _{—} | _{—} | _{—} | _{—} | _{—} | _{—} | V | V | L | G | V | V | F | G | I |
| | A₂ | A₂ | | A_{1c} | A_{1b} | A_{1c} | A₂ | A₃ | | | | | | | A₂ | A₂ | A₂ | | A₂ | A₂ | B₇ | | |
| | A₃ | A₃ | | B₄₄ | A₃ | B₄₄ | A₃ | | | | | | | | B₇ | B₇ | B₇ | | B₇ | B₇ | B₂₇ | | |
| | | | | | | | | | | | | | | | | | B₂₇ | | | | B₄₄ | | |
| | | | | | | | | | | | | | | | | | | | | | A₂₄ | | |

Linker Length in Amino Acids

| Class I MHC | 0 | 1 | 2 | 3 | 4 | 5 | 6 |
|---|---|---|---|---|---|---|---|
| A₂ | 4 | 3 | 1 | 1 | 2 | 2 | 1 |
| A₃ | 0 | 0 | 0 | 0 | 0 | 0 | 0 |
| A_{IC} | 0 | 0 | 0 | 0 | 0 | 0 | 0 |
| A_{1B} | 0 | 0 | 0 | 0 | 0 | 0 | 0 |
| B₄₄ | 0 | 0 | 0 | 0 | 0 | 0 | 0 |
| Total | 4 | 3 | 1 | 1 | 2 | 2 | 1 |

Allowing linkers of 0 to 6 amino acids between epitopes, the 8(N-terminal)-4 (C-terminal) epitope pairing cannot be used in a polyepitope with zero junctional epitopes for the Class I haplotype motifs considered here.

### Example 68

Junctional epitopes between epitopes 8 and 5 as a function of linker length Epitope 8 at N end, Epitope 5 at C end

| | | | | | | | | | | | | | | | | | | | | | | | | |
|---|---|---|---|---|---|---|---|---|---|---|---|---|---|---|---|---|---|---|---|---|---|---|---|---|
| R | L | L | Q | E | T | E | L | V | _{—} | _{—} | _{—} | _{—} | _{—} | _{—} | Y | L | Q | L | V | F | G | I | E | V |
| | A₂ | A₂ | | A_{1c} | A_{1b} | A_{1c} | A₂ | A₃ | | | | | | | A_{1b} | A₂ | | A₂ | A₂ | A₂₄ | | | | |
| | A₃ | A₃ | | B₄₄ | A₃ | B₄₄ | A₃ | | | | | | | A_{1c} | B₇ | | B₇ | B₇ | B₇ | | | | | |
| | | | | | | | | | | | | | | | B₄₄ | B₂₇ | | B₂₇ | | B₄₄ | | | | |
| | | | | | | | | | | | | | | | | | | | | B₂₇ | | | | |

Linker Length in Amino Acids

| Class I MHC | 0 | 1 | 2 | 3 | 4 | 5 | 6 |
|---|---|---|---|---|---|---|---|
| A₂ | 1 | 1 | 2 | 1 | 1 | 1 | 0 |
| A₃ | 0 | 0 | 0 | 0 | 0 | 0 | 0 |
| A_{1C} | 0 | 1 | 1 | 1 | 1 | 0 | 0 |

| | | | | | | | |
|---|---|---|---|---|---|---|---|
| A_{1b} | 0 | 0 | 0 | 1 | 1 | 0 | 0 |
| B₄₄ | 1 | 0 | 1 | 1 | 1 | 1 | 0 |
| Total | 2 | 2 | 4 | 4 | 4 | 2 | 0 |

The 8(N-terminal)-5(C-terminal) epitope pairing can be used in a polyepitope with zero junctional epitopes for the Class I haplotype motifs considered here. Linkers of six amino acids in length will produce no junctional epitopes.

To avoid junctional epitopes, the following amino acids MUST be avoided in a six amino acid linker between epitopes 8(N-terminal)-5(C-terminal). The linker is shown from left to right, with the most N-terminal residue of the linker occuring at the left, and the most C-terminal residue occuring to the right. Amino acids to be avoided are shown position by position in the linker.

| | | | | | | | |
|---|---|---|---|---|---|---|---|
| N-End | _ | _ | _ | _ | _ | _ | C-End |
| | V | V | F | F | F | F | |
| | I | I | Y | Y | Y | Y | |
| | L | L | L | L | P | V | |
| | K | K | M | M | E | I | |
| | R | R | P | P | R | K | |
| | M | Y | R | K | K | R | |
| | P | M | | R | | M | |
| | D | P | | E | | P | |
| | E | | | | | L | |

### Example 69

Junctional epitopes between epitopes 8 and 6 as a function of linker length Epitope 8 at N-end, Epitope 6 at C-end

| | | | | | | | | | | | | | | | | | | | | | | | |
|---|---|---|---|---|---|---|---|---|---|---|---|---|---|---|---|---|---|---|---|---|---|---|---|
| R | L | L | Q | E | T | E | L | V | _{—} | _{—} | _{—} | _{—} | _{—} | _{—} | I | M | I | G | V | L | V | G | V |
| | A₂ | A₂ | | A_{1c} | A₃ | A_{1c} | A₂ | A₃ | | | | | | | A₂ | B₇ | A₂ | | A₂ | A₂ | A₂ | | |
| | A₃ | A₃ | | B₄₄ | A_{1b} | B₄₄ | A₃ | | | | | | | | B₇ | | B₇ | | B₇ | B₇ | B₇ | | |
| | | | | | | | | | | | | | | | | | | | | B₂₇ | | | |

Linker Length in Amino Acids

| Class I MHC | 0 | 1 | 2 | 3 | 4 | 5 | 6 |
|---|---|---|---|---|---|---|---|
| A₂ | 4 | 3 | 1 | 1 | 1 | 1 | 1 |
| A₃ | 0 | 0 | 0 | 0 | 0 | 0 | 0 |
| A_{1c} | 0 | 0 | 0 | 0 | 0 | 0 | 0 |
| A_{1b} | 0 | 0 | 0 | 0 | 0 | 0 | 0 |

| | | | | | | | |
|---|---|---|---|---|---|---|---|
| B₄₄ | 0 | 0 | 0 | 0 | 0 | 0 | 0 |
| Total | 4 | 3 | 1 | 1 | 1 | 1 | 1 |

Allowing linkers of 0 to 6 amino acids between epitopes, the 8(N-terminal)-6 (C-terminal) epitope pairing cannot be used in a polyepitope with zero junctional epitopes for the Class I haplotype motifs considered here.

### Example 70

Junctional epitopes between epitopes 8 and 7 as a function of linker length Epitope 8 at N end, Epitope 7 at C end

| | | | | | | | | | | | | | | | | | | | | | | | |
|---|---|---|---|---|---|---|---|---|---|---|---|---|---|---|---|---|---|---|---|---|---|---|---|
| R | L | L | Q | E | T | E | L | V | _{—} | _{—} | _{—} | _{—} | _{—} | _{—} | Y | L | S | G | A | N | L | N | V |
| | A₂ | A₂ | | A_{1c} | A₃ | A_{1c} | A₂ | A₃ | | | | | | | A_{1b} | A₂ | | | | | A₂ | | |
| | A₃ | A₃ | | B₄₄ | A_{1b} | B₄₄ | A₃ | | | | | | | | A_{1C} | B₇ | | | | | B₇ | | |
| | | | | | | | | | | | | | | | B₄₄ | B₂₇ | | | | | B₂₇ | | |

Linker Length in Amino Acids

| Class I MHC | 0 | 1 | 2 | 3 | 4 | 5 | 6 |
|---|---|---|---|---|---|---|---|
| A₂ | 2 | 0 | 0 | 0 | 1 | 1 | 0 |
| A₃ | 0 | 0 | 0 | 0 | 0 | 0 | 0 |
| A_{1c} | 0 | 1 | 1 | 1 | 1 | 0 | 0 |
| A_{1b} | 0 | 0 | 0 | 1 | 1 | 0 | 0 |
| B₄₄ | 0 | 0 | 1 | 1 | 1 | 1 | 0 |
| Total | 2 | 1 | 2 | 3 | 4 | 2 | 0 |

The 8(N-terminal)-7(C-terminal) epitope pairing can be used in a polyepitope with zero junctional epitopes for the Class I haplotype motifs considered here. Linkers of six amino acids in length will produce no junctional epitopes.

To avoid junctional epitopes, the following amino acids MUST be avoided in a six amino acid linker between epitopes 8(N-terminal)-7(C-terminal). The linker is shown from left to right, with the most N-terminal residue of the linker occuring at the left, and the most C-terminal residue occuring to the right. Amino acids to be avoided are shown position by position in the linker.

| | | | | | | | |
|---|---|---|---|---|---|---|---|
| N-End | _ | _ | _ | _ | _ | _ | C-End |
| | V | V | Y | K | L | V | |
| | I | I | F | R | M | I | |
| | L | L | | Y | P | L | |
| | K | K | | F | Y | K | |
| | R | R | | | F | R | |
| | M | Y | | | K | Y | |
| | P | | | | R | M | |
| | D | | | | | F | |
| | E | | | | | P | |

### Example 71

Junctional epitopes between epitopes 8 and 8 as a function of linker length Epitope 8 at N-end, Epitope 7 at C-end

| | | | | | | | | | | | | | | | | | | | | | | | |
|---|---|---|---|---|---|---|---|---|---|---|---|---|---|---|---|---|---|---|---|---|---|---|---|
| R | L | L | Q | E | T | E | L | V | _{—} | _{—} | _{—} | _{—} | _{—} | _{—} | R | L | L | Q | E | T | E | L | V |
| | A₂ | A₂ | | A_{1c} | A₃ | A_{1c} | A₂ | A₃ | | | | | | | A₃ | A₂ | A₂ | | | | | A₂ | |
| | A₃ | A₃ | | B₄₄ | A_{1b} | B₄₄ | A₃ | | | | | | | | B₂₇ | B₇ | B₇ | | | | | B₇ | |
| | | | | | | | | | | | | | | | | B₂₇ | B₂₇ | | | | | B₂₇ | |

Linker Length in Amino Acids

| Class I MHC | 0 | 1 | 2 | 3 | 4 | 5 | 6 |
|---|---|---|---|---|---|---|---|
| A₂ | 1 | 0 | 0 | 1 | 2 | 1 | 0 |
| A₃ | 2 | 1 | 0 | 1 | 1 | 1 | 2 |
| A_{1b} | 0 | 0 | 0 | 0 | 0 | 0 | 0 |
| A_{1c} | 0 | 0 | 0 | 0 | 0 | 0 | 0 |
| B₄₄ | 0 | 0 | 0 | 0 | 0 | 0 | 0 |
| Total | 3 | 1 | 0 | 2 | 3 | 2 | 2 |

The 8(N-terminal)-8(C-terminal) epitope pairing can be used in a polyepitope with zero junctional epitopes for the Class I haplotype motifs considered here. Linkers of two amino acids in length will produce no junctional epitopes.

To avoid junctional epitopes, the following amino acids MUST be avoided in a two amino acid linker between epitopes 8(N-terminal)-8(C-terminal). The linker is shown from left to right, with the most N-terminal residue of the linker occuring at the left, and the most C-terminal residue occuring to the right. Amino acids to be avoided are shown position by position in the linker

| | | | |
|---|---|---|---|
| N-End | _ | _ | C-End |
| | V | V | |
| | I | I | |
| | L | L | |
| | K | K | |
| | R | R | |
| | | Y | |
| | | M | |
| | | P | |

### Example 72

Junctional epitopes between epitopes 8 and 9 as a function of linker length Epitope 8 at N-end, Epitope 9 at C-end

| | | | | | | | | | | | | | | | | | | | | | | | |
|---|---|---|---|---|---|---|---|---|---|---|---|---|---|---|---|---|---|---|---|---|---|---|---|
| R | L | L | Q | E | T | E | L | V | _{—} | _{—} | _{—} | _{—} | _{—} | _{—} | S | M | P | P | P | G | T | R | V |
| | A₂ | A₂ | | A_{1c} | A₃ | A_{1c} | A₂ | A₃ | | | | | | | | B₇ | | | | | | B₂₇ | |
| | A₃ | A₃ | | B₄₄ | A_{1b} | B₄₄ | A₃ | | | | | | | | | | | | | | | A₃ | |

Linker Length in Amino Acids

| Class I MHC | 0 | 1 | 2 | 3 | 4 | 5 | 6 |
|---|---|---|---|---|---|---|---|
| A₂ | 0 | 0 | 0 | 0 | 0 | 0 | 0 |
| A₃ | 1 | 0 | 0 | 0 | 0 | 0 | 0 |
| A_{IC} | 0 | 0 | 0 | 0 | 0 | 0 | 0 |
| A_{1B} | 0 | 0 | 0 | 0 | 0 | 0 | 0 |
| B₄₄ | 0 | 0 | 0 | 0 | 0 | 0 | 0 |
| Total | 1 | 0 | 0 | 0 | 0 | 0 | 0 |

The 8(N-terminal)-9(C-terminal) epitope pairing can be used in a polyepitope with zero junctional epitopes for the Class I haplotype motifs considered here. Linkers of one, two, three, four, five, or six amino acids in length will produce no junctional epitopes.

To avoid junctional epitopes, the following amino acids MUST be avoided in a one amino acid linker between epitopes 8(N-terminal)-9(C-terminal). The linker is shown from left to right, with the most N-terminal residue of the linker occuring at the left, and the most C-terminal residue occuring to the right. Amino acids to be avoided are shown position by position in the linker.

| | | |
|---|---|---|
| N-End | _{—} | C-End |
| | R | |
| | L | |
| | M | |
| | V | |
| | T | |
| | I | |
| | K | |

### Example 73

Junctional epitopes between epitopes 9 and 1 as a function of linker length Epitope 9 at N end, Epitope 1 at C end

| | | | | | | | | | | | | | | | | | | | | | | | |
|---|---|---|---|---|---|---|---|---|---|---|---|---|---|---|---|---|---|---|---|---|---|---|---|
| S | M | P | P | P | G | T | R | V | _{—} | _{—} | _{—} | _{—} | _{—} | _{—} | K | L | C | P | V | Q | L | W | V |
| | A₂ | B₇ | B₇ | B₇ | | A_{1b} | B₂₇ | A₃ | | | | | | | A₃ | A₂ | | | A₂ | | A₂ | | |
| | A₃ | | | | | A₃ | | | | | | | | | B₂₇ | B₇ | | | B₇ | | B₇ | | |
| | | | | | | | | | | | | | | | | B₂₇ | | | | | B₂₇ | | |

Linker Length in Amino Acids

| Class I MHC | 0 | 1 | 2 | 3 | 4 | 5 | 6 |
|---|---|---|---|---|---|---|---|
| A₂ | 0 | 0 | 0 | 0 | 0 | 0 | 0 |
| A₃ | 1 | 0 | 0 | 0 | 1 | 1 | 1 |
| B₇ | 2 | 2 | 1 | 0 | 0 | 0 | 0 |
| A_{1B} | 0 | 0 | 0 | 0 | 0 | 0 | 0 |
| B₂₇ | 1 | 0 | 0 | 0 | 1 | 2 | 1 |
| | 4 | 2 | 1 | 0 | 2 | 3 | 2 |

The 9(N-terminal)-1(C-terminal) epitope pairing can be used in a polyepitope with zero junctional epitopes for the Class I haplotype motifs considered here. Linkers of three amino acids in length will produce no junctional epitopes.

To avoid junctional epitopes, the following amino acids MUST be avoided in a three amino acid linker between epitopes 9(N-terminal)-1(C-terminal). The linker is shown from left to right, with the most N-terminal residue of the linker occurring at the left, and the most C-terminal residue occurring to the right. Amino acids to be avoided are shown position by position in the linker.

| | | | | |
|---|---|---|---|---|
| N-End | _{—} | _{—} | _{—} | C-End |
| | L | L | L | |
| | M | M | M | |
| | V | P | P | |
| | I | R | R | |
| | K | I | V | |
| | R | V | F | |
| | F | F | I | |
| | P | | | |

### Example 74

Junctional epitopes between epitopes 9 and 2 as a function of linker length Epitope 9 at N end, Epitope 2 at C end

| | | | | | | | | | | | | | | | | | | | | | |
|---|---|---|---|---|---|---|---|---|---|---|---|---|---|---|---|---|---|---|---|---|---|
| S | M | P | P | P | G | T | R | V | _{— — — — — —} | A | K | F | V | A | A | W | T | L | K | A | AA |
| | A₂ | B₇ | B₇ | B₇ | | A₃ | B₂₇ | A₃ | | | A₃ | B₇ | A₂ | | | | | | | | |
| | A₃ | | | | | A_{1b} | | | | | B₂₇ | B₂₇ | B₇ | | | | | | | | |
| | | | | | | | | | | | | A₂₄ | | | | | | | | | |
| | | | | | | | | | | | | B₄₄ | | | | | | | | | |

Linker Length in Amino Acids

| Class I MHC | 0 | 1 | 2 | 3 | 4 | 5 | 6 |
|---|---|---|---|---|---|---|---|
| A₂ | 0 | 0 | 0 | 0 | 0 | 0 | 0 |
| A₃ | 0 | 0 | 0 | 1 | 1 | 1 | 1 |
| B₇ | 3 | 1 | 0 | 0 | 0 | 0 | 0 |
| A_{1b} | 0 | 0 | 0 | 0 | 0 | 0 | 0 |
| B₂₇ | 0 | 0 | 0 | 1 | 2 | 1 | 0 |
| Total | 3 | 1 | 0 | 2 | 3 | 2 | 1 |

The 9(N-terminal)-2(C-terminal) epitope pairing can be used in a polyepitope with zero junctional epitopes for the Class I haplotype motifs considered here. Linkers of two amino acids in length will produce no junctional epitopes.

To avoid junctional epitopes, the following amino acids MUST be avoided in a two amino acid linker between epitopes 9(N-terminal)-2(C-terminal). The linker is shown from left to right, with the most N-terminal residue of the linker occurring at the left, and the most C-terminal residue occurring-to the right. Amino acids to be avoided are shown position by position in the linker.

| | | | |
|---|---|---|---|
| N-End | _{—} | _{—} | C-End |
| | V | L | |
| | I | I | |
| | L | M | |
| | K | V | |
| | R | F | |
| | M | | |
| | F | | |

### Example 75

Junctional epitopes between epitopes 9 and 3 as a function of linker length Epitope 9 at N end, Epitope 3 at C end

| | | | | | | | | | | | | | | | | | | |
|---|---|---|---|---|---|---|---|---|---|---|---|---|---|---|---|---|---|---|
| S | M | P | P | P | G | T | R | V | _{— — — — — —} | K | V | A | E | L | V | H | F | L |
| | A₂ | B₇ | B₇ | B₇ | | A_{1b} | B₂₇ | A₃ | | B₂₇ | A₂ | | | A₂ | A₂ | | B₇ | |
| | A₃ | | | | | A₃ | | | | A₃ | B₇ | | | B₇ | B₇ | | B₂₇ | |
| | | | | | | | | | | | | | | B₂₇ | | | A₂₄ | |
| | | | | | | | | | | | | | | | | | B₄₄ | |

Linker Length in Amino Acids

| Class I MHC | 0 | 1 | 2 | 3 | 4 | 5 | 6 |
|---|---|---|---|---|---|---|---|
| A₂ | 0 | 0 | 0 | 0 | 0 | 0 | 0 |
| A₃ | 1 | 0 | 0 | 0 | 1 | 1 | 1 |
| B₇ | 2 | 2 | 1 | 0 | 0 | 0 | 0 |
| A_{1b} | 0 | 0 | 0 | 0 | 0 | 0 | 0 |
| B₂₇ | 0 | 1 | 1 | 0 | 0 | 1 | 1 |
| Total | 3 | 3 | 2 | 0 | 1 | 2 | 2 |

The 9(N-terminal)-3(C-terminal) epitope pairing can be used in a polyepitope with zero junctional epitopes for the Class I haplotype motifs considered here. Linkers of three amino acids in length will produce no junctional epitopes.

To avoid junctional epitopes, the following amino acids MUST be avoided in a 3 amino acid linker between epitopes 9(N-terminal)-3(C-terminal). The linker is shown from left to right, with the most N-terminal residue of the linker occurring at the left, and the most C-terminal residue occurring to the right. Amino acids to be avoided are shown position by position in the linker.

| | | | | |
|---|---|---|---|---|
| N-End | _{—} | _{—} | _{—} | C-End |
| | V | L | L | |
| | I | I | I | |
| | L | M | M | |
| | K | V | V | |
| | R | F | F | |
| | M | P | P | |
| | F | | Y | |
| | P | | R | |
| | | | E | |

### Example 76

Junctional epitopes between epitopes 9 and 4 as a function of linker length Epitope 9 at N end, Epitope 4 at C end

| | | | | | | | | | | | | | | | | | | |
|---|---|---|---|---|---|---|---|---|---|---|---|---|---|---|---|---|---|---|
| S | M | P | P | P | G | T | R | V | _{— — — — — —} | V | V | L | G | V | V | F | G | L |
| | A₂ | B₇ | B₇ | B₇ | | A_{1b} | B₂₇ | A₃ | | A₂ | A₂ | A₂ | | A₂ | A₂ | B₇ | | |
| | A₃ | | | | | A₃ | | | | B₇ | B₇ | B₇ | | B₇ | B₇ | B₂₇ | | |
| | | | | | | | | | | | | | | B₂₇ | | | | |

Linker Length in Amino Acids

| Class I MHC | 0 | 1 | 2 | 3 | 4 | 5 | 6 |
|---|---|---|---|---|---|---|---|
| A₂ | 1 | 0 | 0 | 0 | 0 | 0 | 0 |
| A₃ | 0 | 0 | 0 | 0 | 0 | 0 | 0 |
| B₇ | 5 | 5 | 3 | 1 | 0 | 0 | 0 |
| A_{1b} | 0 | 0 | 0 | 0 | 0 | 0 | 0 |
| B₂₇ | 1 | 0 | 0 | 1 | 1 | 0 | 0 |
| Total | 6 | 5 | 3 | 2 | 1 | 0 | 0 |

The 9(N-terminal)-4(C-terminal) epitope pairing can be used in a polyepitope with zero junctional epitopes for the Class I haplotype motifs considered here. Linkers of five or six amino acids in length will produce no junctional epitopes.

To avoid junctional epitopes, the following amino acids MUST be avoided in a five amino acid linker between epitopes 9(N-terminal)-4(C-terminal). The linker is shown from left to right, with the most N-terminal residue of the linker occuring at the left, and the most C-terminal residue occuring to the right. Amino acids to be avoided are shown position by position in the linker.

| | | | | | | |
|---|---|---|---|---|---|---|
| N-End | _{—} | _{—} | _{—} | _{—} | _{—} | C-End |
| | L | L | L | L | P | |
| | M | M | M | M | R | |
| | P | P | P | P | E | |
| | R | I | I | R | Y | |
| | V | V | V | E | K | |
| | I | F | F | Y | | |
| | K | | | I | | |
| | F | | | V | | |
| | | | | F | | |

### Example 77

Junctional epitopes between epitopes 9 and 5 as a function of linker length Epitope 9 at N-end, Epitope 5 at C-end

| | | | | | | | | | | | | | | | | | | | |
|---|---|---|---|---|---|---|---|---|---|---|---|---|---|---|---|---|---|---|---|
| S | M | P | P | P | G | T | R | V | _{— — — — — —} | Y | L | Q | L | V | F | G | I | E | V |
| | A₂ | B₇ | B₇ | B₇ | | A_{1b} | B₂₇ | A₃ | | A_{1b} | A₂ | | A₂ | A₂ | A₂₄ | A₂ | | | |
| | A₃ | | | | | A₃ | | | | A_{1c} | B₇ | | B₇ | B₇ | B₇ | | B₇ | | |
| | | | | | | | | | | B₄₄ | B₂₇ | | B₂₇ | | B₄₄ | | | | |
| | | | | | | | | | | | | | | | B₂₇ | | | | |

Linker Length in Amino Acids

| Class I MHC | 0 | 1 | 2 | 3 | 4 | 5 | 6 |
|---|---|---|---|---|---|---|---|
| A₂ | 0 | 0 | 0 | 0 | 0 | 0 | 0 |
| A3 | 0 | 0 | 0 | 0 | 0 | 0 | 0 |
| B₇ | 3 | 2 | 1 | 0 | 0 | 0 | 0 |
| A_{1b} | 0 | 0 | 0 | 0 | 1 | 1 | 0 |
| B₂₇ | 1 | 1 | 1 | 1 | 1 | 1 | 0 |
| Total | 4 | 3 | 2 | 1 | 2 | 2 | 0 |

The 9(N-terminal)-5(C-terminal) epitope pairing can be used in a polyepitope with zero juncitonal epitopes for the Class I haplotype motifs considered here. Linkers of six amino acids in length will produce no junctional epitopes.

To avoid junctional epitopes, the following amino acids MUST be avoided in a six amino acid linker between epitopes 9(N-terminal)-5(C-terminal). The linker is shown from left to right, with the most N-terminal residue of the linker occurring at the left, and the most C-terminal residue occurring to the right. Amino acids to be avoided are shown position by position in the linker.

| | | | | | | | |
|---|---|---|---|---|---|---|---|
| N-End | _{—} | _{—} | _{—} | _{—} | _{—} | _{—} | C-End |
| | V | L | L | L | K | K | |
| | I | I | I | I | R | R | |
| | L | M | M | M | Y | Y | |
| | K | V | V | V | P | L | |
| | R | F | F | F | E | M | |
| | M | P | P | P | | F | |
| | F | R | R | Y | | P | |
| | P | | | R | | | |
| | D | | | E | | | |
| | E | | | | | | |

### Example 78

Junctional epitopes between epitopes 9 and 6 as a function of linker length Epitope 9 at N-end, Epitope 6 at C-end

| | | | | | | | | | | | | | | | | | | | | | | | |
|---|---|---|---|---|---|---|---|---|---|---|---|---|---|---|---|---|---|---|---|---|---|---|---|
| S | M | P | P | P | G | T | R | V | _{—} | _{—} | _{—} | _{—} | _{—} | _{—} | I | M | I | G | V | L | V | G | V |
| | A₂ | B₇ | B₇ | B₇ | | A_{1b} | B₂₇ | A₃ | | | | | | | A₂ | B₇ | A₂ | | A₂ | A₂ | A₂ | | |
| | A₃ | | | | | A₃ | | | | | | | | | B₇ | | B₇ | | B₇ | B₇ | B₇ | | |
| | | | | | | | | | | | | | | | | | | | | B₂₇ | | | |

Linker Length in Amino Acids

| Class I MHC | 0 | 1 | 2 | 3 | 4 | 5 | 6 |
|---|---|---|---|---|---|---|---|
| A₂ | 1 | 0 | 0 | 0 | 0 | 0 | 0 |
| A₃ | 0 | 0 | 0 | 0 | 0 | 0 | 0 |
| B₇ | 5 | 5 | 3 | 1 | 0 | 0 | 0 |
| A_{1b} | 0 | 0 | 0 | 0 | 0 | 0 | 0 |
| B₂₇ | 1 | 1 | 0 | 0 | 0 | 0 | 0 |
| Total | 7 | 6 | 3 | 1 | 0 | 0 | 0 |

The 9(N-terminal)-6(C-terminal) epitope pairing can be used in a polyepitope with zero junctional epitopes for the Class I haplotype motifs considered here. Linkers of four, five, or six amino acids in length will produce no junctional epitopes.

To avoid junctional epitopes, the following amino acids MUST be avoided in a four amino acid linker between epitopes 9(N-terminal)-6(C-terminal). The linker is shown from left to right, with the most N-terminal residue of the linker occuring at the left, and the most C-terminal residue occuring to-the right. Amino acids to be avoided are shown position by position in the linker.

| | | | | | |
|---|---|---|---|---|---|
| N-End | _{—} | _{—} | _{—} | _{—} | C-End |
| | V | L | L | L | |
| | I | I | I | I | |
| | L | M | M | M | |
| | K | V | V | V | |
| | R | F | F | F | |
| | M | P | P | P | |
| | F | R | R | | |
| | P | | | | |

### Example 79

Junctional epitopes between epitopes 9 and 7 as a function of linker length Epitope 9 at N-end, Epitope 7 at C-end

| | | | | | | | | | | | | | | | | | | | | | | | |
|---|---|---|---|---|---|---|---|---|---|---|---|---|---|---|---|---|---|---|---|---|---|---|---|
| S | M | P | P | P | G | T | R | V | _{—} | _{—} | _{—} | _{—} | _{—} | _{—} | Y | L | S | G | A | N | L | N | V |
| | A₂ | B₇ | B₇ | B₇ | | A_{1b} | B₂₇ | A₃ | | | | | | | A_{1b} | A₂ | | | | | A₂ | | |
| | A₃ | | | | | A₃ | | | | | | | | | A_{1c} | B₇ | | | | | B₇ | | |
| | | | | | | | | | | | | | | | B₄₄ | B₂₇ | | | | | B₂₇ | | |

Linker Length in Amino Acids

| Class I MHC | 0 | 1 | 2 | 3 | 4 | 5 | 6 |
|---|---|---|---|---|---|---|---|
| A₂ | 0 | 0 | 0 | 0 | 0 | 0 | 0 |
| A₃ | 0 | 0 | 0 | 0 | 0 | 0 | 0 |
| B₇ | 2 | 2 | 1 | 0 | 0 | 0 | 0 |
| A_{1b} | 0 | 0 | 0 | 0 | 1 | 1 | 0 |
| B₂₇ | 1 | 0 | 0 | 0 | 1 | 1 | 0 |
| Total | 3 | 2 | 1 | 0 | 2 | 2 | 0 |

The 9(N-terminal)-7(C-terminal) epitope pairing can be used in a polyepitope with zero junctional epitopes for the Class I haplotype motifs considered here. Linkers of three or six amino acids in length will produce no junctional epitopes.

To avoid junctional epitopes, the following amino acids MUST be avoided in a three amino acid linker between epitopes 9(N-terminal)-7(C-terminal). The linker is shown from left to right, with the most N-terminal residue of the linker occurring at the left, and the most C-terminal residue occurring to the right. Amino acids to be avoided are shown position by position in the linker.

| | | | | |
|---|---|---|---|---|
| N-End | _{—} | _{—} | _{—} | C-End |
| | V | L | L | |
| | I | I | I | |
| | L | M | M | |
| | K | V | V | |
| | R | F | F | |
| | M | P | P | |
| | F | R | R | |

### Example 80

Junctional epitopes between epitopes 9 and 8 as a function of linker length Epitope 9 at N-end, Epitope 8 at C-end

| | | | | | | | | | | | | | | | | | | | | | | | |
|---|---|---|---|---|---|---|---|---|---|---|---|---|---|---|---|---|---|---|---|---|---|---|---|
| S | M | P | P | P | G | T | R | V | _{—} | _{—} | _{—} | _{—} | _{—} | _{—} | R | L | L | Q | E | T | E | L | V |
| | A₂ | B₇ | B₇ | B₇ | | A_{1b} | B₂₇ | A₃ | | | | | | | A₃ | A₂ | A₂ | | | | | A₂ | |
| | A₃ | | | | | A₃ | | | | | | | | | B₂₇ | B₇ | B₇ | | | | | B₂₇ | |
| | | | | | | | | | | | | | | | | B₂₇ | B₂₇ | | | | | B₇ | |

Linker Length in Amino Acids

| Class I MHC | 0 | 1 | 2 | 3 | 4 | 5 | 6 |
|---|---|---|---|---|---|---|---|
| A₂ | 0 | 0 | 0 | 0 | 0 | 0 | 0 |
| A₃ | 1 | 0 | 0 | 0 | 1 | 1 | 1 |
| B₇ | 4 | 3 | 1 | 0 | 0 | 0 | 0 |
| A_{1b} | 0 | 0 | 0 | 0 | 0 | 0 | 0 |
| B₂₇ | 0 | 0 | 0 | 1 | 2 | 2 | 1 |
| Total | 5 | 3 | 1 | 1 | 3 | 3 | 2 |

Allowing linkers of 0 to 6 amino acids between epitopes, the 9(N-terminal)-8 (C-terminal) epitope pairing cannot be used in a polyepitope with zero junctional epitopes for the Class I haplotype motifs considered here.

### Example 81

Junctional epitopes between epitopes 9 and 9 as a function of linker length Epitope 9 at N-end, Epitope 9 at C-end

| | | | | | | | | | | | | | | | | | | | | | | | |
|---|---|---|---|---|---|---|---|---|---|---|---|---|---|---|---|---|---|---|---|---|---|---|---|
| S | M | P | P | P | G | T | R | V | _{—} | _{—} | _{—} | _{—} | _{—} | _{—} | S | M | P | P | P | G | T | R | V |
| | A₂ | B₇ | B₇ | B₇ | | A_{1b} | B₂₇ | A₃ | | | | | | | | B₇ | | | | | | B₂₇ | |
| | A₃ | | | | | A₃ | | | | | | | | | | | | | | | | A₃ | |

Linker Length in Amino Acids

| Class I MHC | 0 | 1 | 2 | 3 | 4 | 5 | 6 |
|---|---|---|---|---|---|---|---|
| A₂ | 0 | 0 | 0 | 0 | 0 | 0 | 0 |
| A₃ | 1 | 0 | 0 | 0 | 0 | 0 | 0 |
| B₇ | 2 | 2 | 1 | 0 | 0 | 0 | 0 |
| A_{1b} | 0 | 0 | 0 | 0 | 0 | 0 | 0 |
| B₂₇ | 0 | 0 | 0 | 0 | 0 | 0 | 0 |
| Total | 3 | 2 | 1 | 0 | 0 | 0 | 0 |

The 9(N-terminal)-9(C-terminal) epitope pairing can be used in a polyepitope with zero junctional epitopes for the Class I haplotype motifs considered here. Linkers of three, four, five, or six amino acids in length will produce no junctional epitopes.

To avoid junctional epitopes, the following amino acids MUST be avoided in a three amino acid linker between epitopes 9(N-terminal)-9(C-terminal). The linker is shown from left to right, with the most N-terminal residue of the linker occuring at the left, and the most C-terminal residue occuring to the right. Amino acids to be avoided are shown position by position in the linker.

| | | | | |
|---|---|---|---|---|
| N-End | _{—} | _{—} | _{—} | C-End |
| | V | L | L | |
| | I | I | I | |
| | L | M | M | |
| | K | V | V | |
| | R | F | F | |
| | M | | T | |
| | F | | R | |

### Example 82

Epitopes that can be abutted to one another with a linker between them exhibit no junctional epitopes. Epitopes are referred to by the numbers used in Table 5. N-terminal and C-terminal epitopes are indicated. The minimum number of amino acids in a linker that must be inserted between the two epitopes to eliminate junctional epitopes is also listed. The data in Example 82 was compiled from Examples 1-81.

| | | Minimal number of |
|---|---|---|
| N-terminal epitope | C-terminal epitope | amino acids in linker |
| 1 | 4 | 6 |
| 1 | 5 | 5 |
| 1 | 6 | 6 |
| 1 | 7 | 2 |
| 1 | 9 | 2 |
| 2 | 1 | 0 |
| 2 | 3 | 0 |
| 2 | 4 | 4 |
| 2 | 5 | 3 |
| 2 | 6 | 4 |
| 2 | 7 | 0 |
| 2 | 8 | 4 |
| 2 | 9 | 0 |
| 3 | 7 | 3 |
| 3 | 9 | 1 |
| 4 | 1 | 5 |
| 4 | 2 | 4 |
| 4 | 3 | 5 |
| 4 | 5 | 1 |
| 4 | 6 | 2 |
| 4 | 7 | 1 |
| 4 | 8 | 5 |
| 4 | 9 | 0 |
| 5 | 1 | 3 |
| 5 | 2 | 2 |
| 5 | 3 | 3 |
| 5 | 4 | 2 |
| 5 | 6 | 2 |
| 5 | 7 | 1 |
| 5 | 8 | 3 |
| 5 | 9 | 1 |
| 6 | 1 | 1 |
| 6 | 3 | 1 |
| 6 | 4 | 5 |
| 6 | 5 | 4 |
| 6 | 7 | 0 |
| 6 | 9 | 1 |
| 7 | 1 | 2 |
| 7 | 2 | 0 |
| 7 | 3 | 2 |
| 7 | 4 | 6 |
| 7 | 5 | 5 |
| 7 | 6 | 6 |
| 7 | 8 | 1 |
| 7 | 9 | 1 |
| 8 | 5 | 6 |
| 8 | 7 | 6 |
| 8 | 9 | 1 |
| 4 | 4 | 2 |
| 5 | 5 | 2 |
| 6 | 6 | 5 |
| 7 | 7 | 2 |
| 9 | 9 | 3 |
| 8 | 8 | 2 |
| 9 | 1 | 3 |
| 9 | 2 | 2 |
| 9 | 3 | 3 |
| 9 | 4 | 5 |
| 9 | 5 | 6 |
| 9 | 6 | 2 |
| 9 | 7 | 3 |

### Example 83

A polyepitope configuration determined from the data of Examples 1-82. Vaccine epitopes are referred to using the nomenclature of Table 5, and the N-terminal vaccine epitope of the polyepitope is at the left with the remaining vaccine epitopes listed to the right to the C-terminal vaccine epitope, which is shown on the far right. The number of amino acids (aa) in a linker between two vaccine epitopes is shown in parenthesis between the vaccine epitopes. All polyepitopes of this configuration exhibit no junctional epitopes beginning in the N-terminal epitope of a vaccine epitope pair and ending in a C-terminal epitope of a vaccine epitope pair.

5 (2aa) 2 (0aa) 1 (2aa) 7 (1aa) 8 (1aa) 9 (5aa) 4 (2aa) 6 (1aa) 3

### Example 84

A polyepitope configuration determined from the data of Examples 1-82. Vaccine epitopes are referred to using the nomenclature of Table 5, and the N-terminal vaccine epitope of the polyepitope is at the left with the remaining vaccine epitopes listed to the right to the C-terminal vaccine epitope, which is shown on the far right. The number of amino acids (aa) in a linker between two vaccine epitopes is shown in parenthesis between the vaccine epitopes. All polyepitopes of this configuration exhibit no junctional epitopes beginning in the N-terminal epitope of a vaccine epitope pair and ending in a C-terminal epitope of a vaccine epitope pair.

9 (2aa) 2 (0aa) 1 (5aa) 5 (2aa) 4 (2aa) 6 (1aa) 3 (3aa) 7 (1aa) 8

### Example 85

A polyepitope configuration determined from the data of Examples 1-82. Vaccine epitopes are referred to using the nomenclature of Table 5, and the N-terminal vaccine epitope of the polyepitope is at the left with the remaining vaccine epitopes listed to the right to the C-terminal vaccine epitope, which is shown on the far right. The number of amino acids (aa) in a linker between two vaccine epitopes is shown in parenthesis between the vaccine epitopes. All polyepitopes of this configuration exhibit no junctional epitopes beginning in the N-terminal epitope of a vaccine epitope pair and ending in a C-terminal epitope of a vaccine epitope pair.

4 (1aa) 5 (2aa) 6 (1aa) 3 (1aa) 9 (2aa) 2 (0aa) 1 (2aa) 7 (1aa) 8

### Example 86

A specific polyepitope of the configuration set out in Example 83 containing only the vaccine polyepitopes of Table 5. Amino acid content of linkers are listed between the epitope pairs in parenthesis using the one letter code for amino acids. Linker content is listed in order from the most N-terminal to most C-terminal positions left to right. Linkers containing no amino acids are indicated by empty parenthesis.

5 (aa) 2 ( ) 1 (se) 7 (a) 8 (a) 9 (gsykl) 4 (se) 6 (a) 3

### Example 87

A specific polyepitope of the configuration set out in Example 83 containing only the vaccine polyepitopes of Table 5. Amino acid content of linkers are listed between the epitope pairs in parenthesis using the one letter code for amino acids. Linker content is listed in order from the most N-terminal to most C-terminal positions left to right. Linkers containing no amino acids are indicated by empty parenthesis, positions left to right.

5 (ae) 2 ( ) 1 (se) 7 (e) 8 (e) 9 (gsykl) 4 (se) 6 (a) 3

### Example 88

A specific polyepitope of the configuration set out in Example 83 containing only the vaccine polyepitopes of Table 5. Amino acid content of linkers are listed between the epitope pairs in parenthesis using the one letter code for amino acids. Linker content is listed in order from the most N-terminal to most C-terminal positions left to right. Linkers containing no amino acids are indicated by empty parenthesis.

5 (aa) 2 ( ) 1 (sa) 7 (a) 8 (a) 9 (gsykl) 4 (se) 6 (a) 3

### Example 89

A specific polyepitope of the configuration set out in Example 83 containing only the vaccine polyepitopes of Table 5.

Amino acid content of linkers are listed between the epitope pairs in parenthesis using the one letter code for amino acids. Linker content is listed in order from the most N-terminal to most C-terminal positions left to right. Linkers containing no amino acids are indicated by empty parenthesis.

5 (ge) 2 ( ) 1 (te) 7 (a) 8 (a) 9 (arykl) 4 (ge) 6 (a) 3

### Example 90

A specific polyepitope of the configuration set out in Example 83 containing only the vaccine polyepitopes of Table 5. Amino acid content of linkers are listed between the epitope pairs in parenthesis using the one letter code for amino acids. Linker content is listed in order from the most N-terminal to most C-terminal positions left to right. Linkers containing no amino acids are indicated by empty parenthesis.

5 (sa) 2 ( ) 1 (ge) 7 (a) 8 (a) 9 (tggkl) 4 (ge) 6 (a) 3

### Example 91

A specific polyepitope of the configuration set out in Example 84 containing only the vaccine polyepitopes of Table 5. Amino acid content of linkers are listed between the epitope pairs in parenthesis using the one letter code for amino acids. Linker content is listed in order from the most N-terminal to most C-terminal positions left to right. Linkers containing no amino acids are indicated by empty parenthesis.

9 (gg) 2 ( ) 1 (akqla) 5 (gg) 4 (ge) 6 (g) 3 (aka) 7 (g) 8

### Example 92

A specific polyepitope of the configuration set out in Example 84 containing only the vaccine polyepitopes of Table 5. Amino acid content of linkers are listed between the epitope pairs in parenthesis using the one letter code for amino acids. Linker content is listed in order from the most N-terminal to most C-terminal positions left to right. Linkers containing no amino acids are indicated by empty parenthesis.

9 (gg) 2 ( ) 1 (akqla) 5 (gg) 4 (ge) 6 (g) 3 (aea) 7 (g) 8

### Example 93

A specific polyepitope of the configuration set out in Example 84 contains only the vaccine polyepitopes of Table 5. Amino acid content of linkers are listed between the epitope pairs in parenthesis using the one letter code for amino acids. Linker content is liusted in order from the most N-terminal to most C-terminal positions left to right. Linkers containing no amino acids are indicated by empty parenthesis.

9 (gg) 2 ( ) 1 (aelqa) 5 (gg) 4 (ge) 6 (g) 3 (aea) 7 (g) 8

### Example 94

A specific polyepitope of the configuration set out in Example 84 containing only the vaccine polyepitopes of Table 5. Amino acid content of linkers are listed between the epitope pairs in parenthesis using the one letter code for amino acids. Linker content is listed in order from the most N-terminal to most C-terminal positions left to right. Linkers containing no amino acids are indicated by empty parenthesis.

9 (ae) 2 ( ) 1 (akdla) 5 (ae) 4 (ae) 6 (a) 3 (aea) 7 (a) 8

### Example 95

A specific polyepitope of the configuration set out in Example 96 contains only the vaccine polyepitopes of Table 5. Amino acid content of linkers are listed between the epitope pairs in parenthesis using the one letter code for amino acids. Linker content is liusted in order from the most N-terminal to most C-terminal positions left to right. Linkers containing no amino acids are indicated by empty parenthesis.

9 (aa) 2 ( ) 1 (akqla) 5 (aa) 4 (ae) 6 (a) 3 (aka) 7 (a) 8

### Example 96

A specific polyepitope of the configuration set out in Example 84 containing only the vaccine polyepitopes of Table 5. Amino acid content of linkers are listed between the epitope pairs in parenthesis using the one letter code for amino acids. Linker content is listed in order from the most N-terminal to most C-terminal positions left to right. Linkers containing no amino acids are indicated by empty parenthesis.

4 (e) 5(ae) 6(a) 3(a) 9(gr) 2( ) 1 (se) 7(a) 8

### Example 97

A specific polyepitope of the configuration set out in Example 85 containing only the vaccine polyepitopes of Table 5. Amino acid content of linkers are listed between the epitope pairs in parenthesis using the one letter code for amino acids. Linker content is listed in order from the most N-terminal to most C-terminal positions left to right. Linkers containing no amino acids are indicated by empty parenthesis.

4 (e) 5 (ae) 6 (a) 3 (a) 9 (gr) 2 ( ) 1 (se) 7 (a) 8

### Example 98

A specific polyepitope of the configuration set out in Example 85 containing only the vaccine polyepitopes of Table 5. Amino acid content of linkers are listed between the epitope pairs in parenthesis using the one letter code for amino acids. Linker content is listed in order from the most N-terminal to most C-terminal positions left to right. Linkers containing no amino acids are indicated by empty parenthesis.

4 (a) 5 (ae) 6 (a) 3 (a) 9 (gr) 2 ( ) 1 (se) 7 (a) 8

### Example 99

A specific polyepitope of the configuration set out in Example 85 containing only the vaccine polyepitopes of Table 5. Amino acid content of linkers are listed between the epitope pairs in parenthesis using the one letter code for amino acids. Linker content is listed in order from the most N-terminal to most C-terminal positions left to right. Linkers containing no amino acids are indicated by empty parenthesis.

4 (e) 5 (te) 6 (a) 3 (a) 9 (ae) 2 ( ) 1 (ge) 7 (a) 8

### Example 100

A specific polyepitope of the configuration set out in Example 85 containing only the vaccine polyepitopes of Table 5. Amino acid content of linkers are listed between the epitope pairs in parenthesis using the one letter code for amino acids. Linker content is listed in order from the most N-terminal to most C-terminal positions left to right. Linkers containing no amino acids are indicated by empty parenthesis.

4 (e) 5 (ta) 6 (a) 3 (a) 9 (tr) 2 ( ) 1 (se) 7 (a) 8

We claim:

1. A method of eliminating or creating epitopes in a polypeptide comprising pattern-matching a MHC binding motif to the polypeptide, and inserting or deleting amino acids to alter the pattern-match.

2. The method of claim 1, wherein the inserted or deleted amino acids comprise amino acids contained between the N-termus and the C-terminus of the polypeptide.

3. The method of claim 1, wherein the MHC binding motif is selected from the group consisting of A*010101, A*020101, A*0203, A*02l 1, A*030101, A*002, A*1101010, A*1102, A*1103, A*24020101, A*2601, A*2602, A*2902, A*3002, and A*3001.

4. A method of eliminating or creating epitopes in a polypeptide comprising pattern-matching a MHC binding motif to the polypeptide, and changing the amino acid in an anchor selected from the group consisting of N-terminal anchor, C-terminal anchor, and intermediate anchor.

5. A method of selecting the length of a linker between a first polypeptide and a second polypeptide to eliminate junctional epitopes of a MHC binding motif, comprising the steps:
a) pattern-matching the MHC binding motif to the first polypeptide and the second polypeptide;
b) adding amino acids consecutively between the first and second polypeptides until binding motif is altered.

6. A method of eliminating or creating epitopes in a polypeptide comprising the steps:
a) identifying structure-degrading amino acids in the polypeptide;
b) pattern-matching the non-structure degrading amino acids in the polypeptide;
c) changing non-structure degrading amino acids in the polypeptide to alter the pattern-match.

7. A programmed computer, comprising:
a) a means for pattern-matching an MHC binding motif to a symbolic polypeptide;
b) a means for adding or subtracting symbolic amino acids to alter the pattern-match.

8. A programmed computer, comprising:
c) a means for pattern-matching an MHC binding motif to a symbolic polypeptide;
d) a means for changing amino acids in the polypeptide to alter the pattern-match.
